(19) United States
(12) Patent Application Publication
(10) Pub. No.: US 2024/0034807 A1
(43) Pub. Date: Feb. 1, 2024

(54) MOLECULES THAT BIND TO MESOTHELIN POLYPEPTIDES

(71) Applicant: University of Pittsburgh - Of the Commonwealth System of Higher Education, Pittsburgh, PA (US)

(72) Inventors: Dimiter Stanchev Dimitrov, Pittsburgh, PA (US); Zehua Sun, Pittsburgh, PA (US); John W. Mellors, Pittsburgh, PA (US)

(21) Appl. No.: 18/265,125

(22) PCT Filed: Feb. 15, 2022
(86) PCT No.: PCT/US22/16481

§ 371 Date: Jun. 2, 2023

Related U.S. Application Data
(60) Provisional application No. 63/150,008, filed on Feb. 16, 2021.

(51) Int. Cl. C07K 16/30; C07K 14/725; C07K 16/28

(52) U.S. Cl. CPC C07K 16/30; C07K 14/7051; C07K 16/2809; C07K 16/283; C07K 16/2803; C07K 2319/30; C07K 2319/03; C07K 2317/622; C07K 2319/02; C07K 2317/31

(57) ABSTRACT

This document provides methods and materials involved in binding a binder (e.g., an antibody, antigen binding fragment, antibody domain, CAR, cell engager, and/or ADC) to a mesothelin polypeptide. For example, binders (e.g., antibodies, antigen binding fragments, antibody domains, CARs, cell engagers, and/or ADCs) that bind to a mesothelin polypeptide and methods and materials for using one or more such binding molecules to treat a mammal (e.g., a human) having cancer are provided.

### CROSS-REFERENCE TO RELATED APPLICATIONS

This application claims the benefit of U.S. Provisional Application Ser. No. 63/150,008, filed Feb. 16, 2021. The disclosure of the prior application is considered part of (and is incorporated by reference in) the disclosure of this application.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a mesothelin polypeptide. For example, this document provides binders (e.g., antibodies, antigen binding fragments, antibody domains, CARs, cell engagers, or ADCs) that bind to a mesothelin polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a mesothelin polypeptide and methods and materials for using such cells to treat cancer.

### 2. Background Information

Mesothelin is a differentiation antigen that is normally expressed by mesothelial cells in the pleura, pericardium, and peritoneum. Recent studies demonstrated that mesothelin is highly expressed in several human cancers, including mesotheliomas, pancreatic adenocarcinomas, approximately 70% of ovarian cancers, and 50% of lung adenocarcinomas (Hassan et al., J. Clin. Oncol., 34(34):4171-4179 (2016); and Hassan et al., Eur. J. Cancer, 44(1):46-53 (2008)). The mesothelin gene encodes a 71-KD precursor, which is processed to a soluble 31-kDa N-terminal protein called megakaryocyte potentiating factor (MPF) and a 40-kDa membrane-bound fragment called mesothelin. Mesothelin is a 40 kDa, glycosylphosphatidylinositol (GPI) anchored membrane surface glycoprotein.

Amatuximab, BAY94-9343 (also known Anetumab Ravtasine), and DMOT4039A, which target mesothelin, were developed to treat mesothelin positive tumors such as mesothelioma, ovarian, pancreatic, and lung adenocarcinomas (Hassan et al., Clin. Cancer Res., 20(23):5927-5936 (2014); Golfier et al., Mol. Cancer Ther., 13(6):1537-1548 (2014); and Weekes et al., Mol. Cancer Ther., 15(3):439-447 (2016)).

### SUMMARY

This document provides methods and materials involved in binding a molecule (e.g., an antibody, an antigen binding fragment, an antibody domain, a CAR, a cell engager, or an ADC) to a mesothelin polypeptide. For example, this document provides binders (e.g., antibodies, antigen binding fragments, antibody domains, CARs, cell engagers, or ADCs) that bind to a mesothelin polypeptide and methods and materials for using one or more such binders to treat a mammal (e.g., a human) having cancer.

This document also provides cells (e.g., host cells) designed to express one or more binders (e.g., antibodies, antigen binding fragments, antibody domains, CARs, or cell engagers) having the ability to bind to a mesothelin polypeptide and methods and materials for using such cells to treat cancer.

As described herein, binders (e.g., one or more antibodies, one or more antigen binding fragments, one or more antibody domains, one or more CARs, one or more cell engagers, and/or one or more ADCs) can be designed to have the ability to bind to a mesothelin polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human mesothelin polypeptide as set forth in SEQ ID NO:97 or SEQ ID NO:531 (see, e.g., FIG. 1).

In some cases, a single set of three complementarity-determining regions (CDRs) of an antibody domain (e.g., a VH domain) provided herein (e.g., SEQ ID NOs:1-3; SEQ ID NOs:9-11; SEQ ID NOs:17-19; SEQ ID NOs:25-27; SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; or SEQ ID NOs:89-91) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target mesothelin⁺ cells (e.g., mesothelin⁺ tumor cells and/or mesothelin⁺ tumor vasculature), can be engineered into an antibody structure that includes an Fc region to create antibodies having the ability to target mesothelin⁺ cells (e.g., mesothelin⁺ tumor cells and/or mesothelin⁺ tumor vasculature) and induce antibody-dependent cell-mediated cytotoxicity (ADCC) against the target mesothelin⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target mesothelin⁺ cells (e.g., mesothelin⁺ tumor cells and/or mesothelin⁺ tumor vasculature) and induce one or more immune responses (e.g., T cell immune responses and/or ADCC using a cell engager in the absence of an Fc-containing antibody) against the target mesothelin⁺ cells. It is noted that BiKE- and TriKE-mediated killing can be referred to ADCC even though it is not initiated by an Fc domain.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, and/or antibody domain-drug conjugates can be designed to include an appropriate binder provided herein to create the conjugate. Such conjugates can be used to deliver the drug payload to target cells such as cancer cells (e.g., mesothelin⁺ cancer cells) or cancer vasculature (e.g., mesothelin⁺ cancer vasculature).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a mesothelin⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

As also described herein, cells (e.g., host cells) can be designed to express one or more binders (e.g., antibodies, antigen binding fragments, antibody domains, CARs, or cell engagers) having the ability to bind to a mesothelin polypeptide. For example, cells such as T cells (e.g., CTLs), stem cells (e.g., induced pluripotent stem cells), or NK cells can be engineered to express one or more CARs having the ability to bind to a mesothelin polypeptide. Such cells (e.g., mesothelin-specific CAR⁺ T cells or NK cells) can be used to treat cancer.

In some cases, a binder (e.g., an antibody, antigen binding fragment, and/or antibody domain) provided herein can be used to detect the presence or absence of a mesothelin polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains mesothelin⁺ cells (e.g., mesothelin⁺ cancer cells). Having the ability to detect the presence or absence of a mesothelin polypeptide (e.g., mesothelin⁺ cancer cells) can allow clinicians, health professionals, and patients to make better decisions about possible treatment options. For example, detection of mesothelin⁺ cancer cells within a mammal can allow clinicians, health professionals, and patients to select an appropriate anti-cancer treatment that targets the mesothelin⁺ cancer cells. Such treatments that targets the mesothelin⁺ cancer cells can include administration of an anti-mesothelin antibody such as amatuximab, BAY94-9343, and DMOT4039A and/or one or more of the binders described herein having the ability to bind to a mesothelin polypeptide and/or administration of one or more cells (e.g., mesothelin-specific CAR⁺ T cells or NK cells) designed to express a binder described herein.

In general, a first aspect of this document features an antibody comprising (consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions); (ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions); (iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (v) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions); (vi) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (vii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (viii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (ix) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (x) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (xi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); or (xii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions). The antibody can comprise the ability to bind to SEQ ID NO:97 or SEQ ID NO:531. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the heavy chain variable domain or region of the (ii). The heavy chain variable domain or region can comprise an amino acid sequence having at least 90 percent identity to the amino acid sequence set forth in SEQ ID NO:16. The antibody can comprise the heavy chain variable domain or region of the (iii). The heavy chain variable domain or region can comprise an amino acid sequence having at least 90 percent identity to the amino acid sequence set forth in SEQ ID NO:24. The antibody can comprise the heavy chain variable domain or region of the (iv). The heavy chain variable domain or region can comprise an amino acid sequence having at least 90 percent identity to the amino acid sequence set forth in SEQ ID NO:32. The antibody can comprise the heavy chain variable domain or region of the (v). The heavy chain variable domain or region can comprise an amino acid sequence having at least 90 percent identity to the amino acid sequence set forth in SEQ ID NO:40. The antibody can comprise the heavy chain variable domain or region of the (vi). The heavy chain variable domain or region can comprise an amino acid sequence having at least 90 percent identity to the amino acid sequence set forth in SEQ ID NO:48. The antibody can comprise the heavy chain variable domain or region of the (vii). The heavy chain variable domain or region can comprise an amino acid sequence having at least 90 percent identity to the amino acid sequence set forth in SEQ ID NO:56. The antibody can comprise the heavy chain variable domain or region of the (viii). The heavy chain variable domain or region can comprise an amino acid sequence having at least 90 percent identity to the amino acid sequence set forth in SEQ ID NO:64. The antibody can comprise the heavy chain variable domain or region of the (ix). The heavy chain variable domain or region can comprise an amino acid sequence having at least 90 percent identity to the amino acid sequence set forth in SEQ ID NO:72. The antibody can comprise the heavy chain variable domain or region of the (x). The heavy chain variable domain or region can comprise an amino acid sequence having at least 90 percent identity to the amino acid sequence set forth in SEQ ID NO:80. The antibody can comprise the heavy chain variable domain or region of the (xi). The heavy chain variable domain or region can comprise an amino acid sequence having at least 90 percent identity to the amino acid sequence set forth in SEQ ID NO:88. The antibody can comprise the heavy chain variable domain or region of the (xii). The heavy chain variable domain or region can comprise an amino acid sequence having at least 90 percent identity to the amino acid sequence set forth in SEQ ID NO:96. The antibody can be a monoclonal antibody. The antibody can be an scFv antibody.

In a second aspect, this document features an antigen binding fragment comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions); (ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions); (iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (v) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions); (vi) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (vii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (viii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (ix) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (x) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (xi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); or (xii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions). The antigen binding fragment can comprise the ability to bind to SEQ ID NO:97 or SEQ ID NO:531. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the heavy chain variable domain or region of the (ii). The heavy chain variable domain or region can comprise an amino acid sequence having at least 90 percent identity to the amino acid sequence set forth in SEQ ID NO:16. The antigen binding fragment can comprise the heavy chain variable domain or region of the (iii). The heavy chain variable domain or region can comprise an amino acid sequence having at least 90 percent identity to the amino acid sequence set forth in SEQ ID NO:24. The antigen binding fragment can comprise the heavy chain variable domain or region of the (iv). The heavy chain variable domain or region can comprise an amino acid sequence having at least 90 percent identity to the amino acid sequence set forth in SEQ ID NO:32. The antigen binding fragment can comprise the heavy chain variable domain or region of the (v). The heavy chain variable domain or region can comprise an amino acid sequence having at least 90 percent identity to the amino acid sequence set forth in SEQ ID NO:40. The antigen binding fragment can comprise the heavy chain variable domain or region of the (vi). The heavy chain variable domain or region can comprise an amino acid sequence having at least 90 percent identity to the amino acid sequence set forth in SEQ ID NO:48. The antigen binding fragment can comprise the heavy chain variable domain or region of the (vii). The heavy chain variable domain or region can comprise an amino acid sequence having at least 90 percent identity to the amino acid sequence set forth in SEQ ID NO:56. The antigen binding fragment can comprise the heavy chain variable domain or region of the (viii). The heavy chain variable domain or region can comprise an amino acid sequence having at least 90 percent identity to the amino acid sequence set forth in SEQ ID NO:64. The antigen binding fragment can comprise the heavy chain variable domain or region of the (ix). The heavy chain variable domain or region can comprise an amino acid sequence having at least 90 percent identity to the amino acid sequence set forth in SEQ ID NO:72. The antigen binding fragment can comprise the heavy chain variable domain or region of the (x). The heavy chain variable domain or region can comprise an amino acid sequence having at least 90 percent identity to the amino acid sequence set forth in SEQ ID NO:80. The antigen binding fragment comprises the heavy chain variable domain or region of the (xi). The heavy chain variable domain or region can comprise an amino acid sequence having at least 90 percent identity to the amino acid sequence set forth in SEQ ID NO:88. The antigen binding fragment can comprise the heavy chain variable domain or region of the (xii). The heavy chain variable domain or region can comprise an amino acid sequence having at least 90 percent identity to the amino acid sequence set forth in SEQ ID NO:96. The antigen binding fragment can be monoclonal. The antigen binding fragment can be an Fab.

In a third aspect, this document features an antibody domain comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions); (ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions); (iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions); (iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (v) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions); (vi) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (vii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (viii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (ix) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (x) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (xi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); or (xii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions). The antibody domain can comprise the ability to bind to SEQ ID NO:97 or SEQ ID NO:531. The antibody domain can comprise the heavy chain variable domain or region of the (i). The heavy chain variable domain or region can comprise an amino acid sequence having at least 90 percent identity to the amino acid sequence set forth in SEQ ID NO:8. The antibody domain can comprise the heavy chain variable domain or region of the (ii). The heavy chain variable domain or region can comprise an amino acid sequence having at least 90 percent identity to the amino acid sequence set forth in SEQ ID NO:16. The antibody domain can comprise the heavy chain variable domain or region of the (iii). The heavy chain variable domain or region can comprise an amino acid sequence having at least 90 percent identity to the amino acid sequence set forth in SEQ ID NO:24. The antibody domain can comprise the heavy chain variable domain or region of the (iv). The heavy chain variable domain or region can comprise an amino acid sequence having at least 90 percent identity to the amino acid sequence set forth in SEQ ID NO:32. The antibody domain can comprise the heavy chain variable domain or region of the (v). The heavy chain variable domain or region can comprise an amino acid sequence having at least 90 percent identity to the amino acid sequence set forth in SEQ ID NO:40. The antibody domain can comprise the heavy chain variable domain or region of the (vi). The heavy chain variable domain or region can comprise an amino acid sequence having at least 90 percent identity to the amino acid sequence set forth in SEQ ID NO:48. The antibody domain can comprise the heavy chain variable domain or region of the (vii). The heavy chain variable domain or region can comprise an amino acid sequence having at least 90 percent identity to the amino acid sequence set forth in SEQ ID NO:56. The antibody domain can comprise the heavy chain variable domain or region of the (viii). The heavy chain variable domain or region can comprise an amino acid sequence having at least 90 percent identity to the amino acid sequence set forth in SEQ ID NO:64. The antibody domain can comprise the heavy chain variable domain or region of the (ix). The heavy chain variable domain or region can comprise an amino acid sequence having at least 90 percent identity to the amino acid sequence set forth in SEQ ID NO:72. The antibody domain can comprise the heavy chain variable domain or region of the (x). The heavy chain variable domain or region can comprise an amino acid sequence having at least 90 percent identity to the amino acid sequence set forth in SEQ ID NO:80. The antibody domain can comprise the heavy chain variable domain or region of the (xi). The heavy chain variable domain or region can comprise an amino acid sequence having at least 90 percent identity to the amino acid sequence set forth in SEQ ID NO:88. The antibody domain can comprise the heavy chain variable domain or region of the (xii). The heavy chain variable domain or region can comprise an amino acid sequence having at least 90 percent identity to the amino acid sequence set forth in SEQ ID NO:96. The antibody domain can be monoclonal. The antibody domain can be a VH domain.

In another aspect, this document features a chimeric antigen receptor comprising (or consisting essentially of, or consisting of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18.

In another aspect, this document features a cell comprising a chimeric antigen receptor that comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The cell can be a T cell, a stem cell, or an NK cell.

In another aspect, this document features an isolated population of cells, wherein at least one cell of the population comprises a chimeric antigen receptor that comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The cell can be a T cell, a stem cell, or an NK cell. In some embodiments, at least 50 percent, at least 75 percent, at least 95 percent, at least 99 percent, or 100 percent of the cells of the population can comprise the chimeric antigen receptor.

In another aspect, this document features a cell engager comprising (or consisting essentially of, or consisting of) a first antigen binding domain, a linker, and a second antigen binding domain, wherein the antigen binding domain comprises (or consists essentially of, or consists of) an antibody of the first aspect described above, an antigen-binding fragment of the second aspect described above, or an antibody domain of the third aspect described above. The first antigen binding domain can comprise a scFv having the ability to bind to a mesothelin polypeptide. The first antigen binding domain can comprise a VH domain having the ability to bind to a mesothelin polypeptide. The linker can comprise a linker set forth in FIG. 15 or FIG. 16. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 20. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 20.

In another aspect, this document features a nucleic acid comprising (or consisting essentially of, or consisting of) a nucleic acid sequence encoding at least part of an antibody of the first aspect described above, an antigen-binding fragment of the second aspect described above, or an antibody domain of the third aspect described above. The nucleic acid sequence can encode the heavy chain variable domain or region of any one of the (i)-(xii) of the first aspect described above. The nucleic acid can be a viral vector. The nucleic acid can be a phagemid.

In another aspect, this document features a nucleic acid comprising (or consisting essentially of, or consisting of) a nucleic acid sequence encoding a chimeric antigen receptor, wherein the chimeric antigen receptor comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The nucleic acid can be a viral vector. The nucleic acid can be a phagemid.

In another aspect, this document features a nucleic acid comprising (or consisting essentially of, or consisting of) a nucleic acid sequence encoding a cell engager, wherein the cell engager comprises (or consists essentially of, or consists of) a first antigen binding domain, a linker, and a second antigen binding domain, wherein the antigen binding domain comprises (or consists essentially of, or consists of) an antibody of the first aspect described above, an antigen-binding fragment of the second aspect described above, or an antibody domain of the third aspect described above. The first antigen binding domain can comprise a scFv having the ability to bind to a mesothelin polypeptide. The first antigen binding domain can comprise a VH domain having the ability to bind to a mesothelin polypeptide. The linker can comprise a linker set forth in FIG. 15 or FIG. 16. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 20. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 20. The nucleic acid can be a viral vector. The nucleic acid can be a phagemid.

In another aspect, this document features a host cell comprising a nucleic acid of either of the two preceding paragraphs. In another aspect, this document features an isolated population of cells, wherein at least one cell of the population comprises a nucleic acid of either of the two preceding paragraphs. In some embodiments, at least 50 percent, at least 75 percent, at least 95 percent, at least 99 percent, or 100 percent of the cells of the population can comprise a nucleic acid of either of the two preceding paragraphs.

In another aspect, this document features a host cell that expresses a chimeric antigen receptor, wherein the chimeric antigen receptor comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The host cell can be a T cell, stem cell, or NK cell.

In another aspect, this document features an isolated population of host cells, wherein at least one host cell of the population expresses a chimeric antigen receptor, wherein the chimeric antigen receptor comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The host cell can be a T cell, stem cell, or NK cell. In some embodiments, at least 50 percent, at least 75 percent, at least 95 percent, at least 99 percent, or 100 percent of the host cells of the population can express the chimeric antigen receptor.

In another aspect, this document features a host cell that expresses a cell engager, wherein the cell engager comprises (or consists essentially of, or consists of) a first antigen binding domain, a linker, and a second antigen binding domain, wherein the antigen binding domain comprises (or consists essentially of, or consists of) an antibody of the first aspect described above, an antigen-binding fragment of the second aspect described above, or an antibody domain of the third aspect described above. The first antigen binding domain can comprise a scFv having the ability to bind to a mesothelin polypeptide. The first antigen binding domain can comprise a VH domain having the ability to bind to a mesothelin polypeptide. The linker can comprise a linker set forth in FIG. 15 or FIG. 16. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 20. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 20. The host cell can be a T cell, stem cell, or NK cell.

In another aspect, this document features an isolated population of host cells, wherein at least one host cell of the population expresses a cell engager, wherein the cell engager comprises (or consists essentially of, or consists of) a first antigen binding domain, a linker, and a second antigen binding domain, wherein the antigen binding domain comprises (or consists essentially of, or consists of) an antibody of the first aspect described above, an antigen-binding fragment of the second aspect described above, or an antibody domain of the third aspect described above. The first antigen binding domain can comprise a scFv having the ability to bind to a mesothelin polypeptide. The first antigen binding domain can comprise a VH domain having the ability to bind to a mesothelin polypeptide. The linker can comprise a linker set forth in FIG. 15 or FIG. 16. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 20. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 20. The host cell can be a T cell, stem cell, or NK cell. In some embodiments, at least 50 percent, at least 75 percent, at least 95 percent, at least 99 percent, or 100 percent of the host cells of the population can express the cell engager.

In another aspect, this document features an antibody-drug conjugate (ADC) comprising (or consisting essentially of, or consisting of) an antigen binging domain covalently linked to a drug, wherein the antigen binging domain comprises an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The drug can be selected from the group consisting of auristatins, mertansine, or pyrrolobenzodiazepine (PBD) dimers.

In another aspect, this document features a composition comprising (or consisting essentially of, or consisting of) an antibody of the first aspect described above, an antigen-binding fragment of the second aspect described above, or an antibody domain of the third aspect described above. The composition can comprise an antibody of the first aspect described above. The composition comprises an antigen-binding fragment of the second aspect described above. The composition can comprise an antibody domain of the third aspect described above. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of, or consisting of) a cell engager that comprises (or consists essentially of, or consists of) a first antigen binding domain, a linker, and a second antigen binding domain, wherein the antigen binding domain comprises (or consists essentially of, or consists of) an antibody of the first aspect described above, an antigen-binding fragment of the second aspect described above, or an antibody domain of the third aspect described above. The first antigen binding domain can comprise a scFv having the ability to bind to a mesothelin polypeptide. The first antigen binding domain can comprise a VH domain having the ability to bind to a mesothelin polypeptide. The linker can comprise a linker set forth in FIG. 15 or FIG. 16. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 20. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 20. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of, or consisting of) a cell comprising a chimeric antigen receptor that comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The cell can be a T cell, a stem cell, or an NK cell. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of, or consisting of) a cell that expresses a chimeric antigen receptor, wherein the chimeric antigen receptor comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The host cell can be a T cell, stem cell, or NK cell. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a composition comprising (or consisting essentially of, or consisting of) an ADC comprising (or consisting essentially of, or consisting of) an antigen binging domain covalently linked to a drug, wherein the antigen binging domain comprises an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The drug can be selected from the group consisting of auristatins, mertansine, or pyrrolobenzodiazepine (PBD) dimers. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer. The method comprises (or consists essentially of, or consists of) administering, to the mammal, a composition of any of the preceding five paragraphs. The mammal can be a human. The cancer can be a mesothelin⁺ cancer. The mesothelin⁺ cancer can be selected from the group consisting of mesothelin⁺ mesothelioma cancer, mesothelin⁺ ovarian cancer, mesothelin⁺ pancreatic cancer, mesothelin⁺ lung cancer and mesothelin⁺ cholangiocarcinoma. The number of cancer cells within the mammal can be reduced following the administering step.

In another aspect, this document features a method of treating a mammal having cancer. The method comprises (or consists essentially of, or consists of) (a) administering, to the mammal, a composition of any of those preceding five paragraphs, and (b) administering, to the mammal, a composition comprising a checkpoint inhibitor. The mammal can be a human. The method of any one of claims 135-136, wherein the cancer can be a mesothelin⁺ cancer. The method of claim 137, wherein the mesothelin⁺ cancer can be selected from the group consisting of mesothelin⁺ mesothelioma cancer, mesothelin⁺ ovarian cancer, mesothelin⁺ pancreatic cancer, mesothelin⁺ lung cancer and mesothelin⁺ cholangiocarcinoma. The method of any one of claims 135-138, wherein the checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab. The method of any one of claims 135-139, wherein the number of cancer cells within the mammal can be reduced following the administering steps (a) and (b).

In another aspect, this document features a method for binding a binding molecule to a mesothelin polypeptide. The method comprises (or consists essentially of, or consists of) contacting the mesothelin polypeptide with an antibody of the first aspect described above, an antigen-binding fragment of the second aspect described above, or an antibody domain of the third aspect described above. The contacting can be performed in vitro. The contacting can be performed in vivo. The contacting can be performed within a mammal by administering the antibody, the antigen binding fragment, or the antibody domain to the mammal. The mammal can be a human.

In another aspect, this document features a method for binding a binding molecule to a mesothelin polypeptide. The method comprises (or consists essentially of, or consists of) contacting the mesothelin polypeptide with a chimeric antigen receptor, wherein the chimeric antigen receptor comprises (or consists essentially of, or consists of) an antigen binding domain, a hinge, a transmembrane domain, and one or more signaling domains, wherein the antigen binding domain comprises (or consists essentially of, or consists of) an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The hinge can comprise a hinge set forth in FIG. 16. The transmembrane domain can comprise a transmembrane domain set forth in FIG. 17. The chimeric antigen receptor can comprise one or more signaling domains set forth in FIG. 18. The contacting can be performed in vitro. The contacting can be performed in vivo. The contacting can be performed within a mammal by administering the chimeric antigen receptor, the cell engager, or the ADC to the mammal. The mammal can be a human.

In another aspect, this document features a method for binding a binding molecule to a mesothelin polypeptide. The method comprises (or consists essentially of, or consists of) contacting the mesothelin polypeptide with a cell engager, wherein the cell engager comprises (or consists essentially of, or consists of) a first antigen binding domain, a linker, and a second antigen binding domain, wherein the antigen binding domain comprises (or consists essentially of, or consists of) an antibody of the first aspect described above, an antigen-binding fragment of the second aspect described above, or an antibody domain of the third aspect described above. The first antigen binding domain can comprise a scFv having the ability to bind to a mesothelin polypeptide. The first antigen binding domain can comprise a VH domain having the ability to bind to a mesothelin polypeptide. The linker can comprise a linker set forth in FIG. 15 or FIG. 16. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 19. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 20. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 20. The contacting can be performed in vitro. The contacting can be performed in vivo. The contacting can be performed within a mammal by administering the chimeric antigen receptor, the cell engager, or the ADC to the mammal. The mammal can be a human.

In another aspect, this document features a method for binding a binding molecule to a mesothelin polypeptide. The method comprises (or consists essentially of, or consists of) contacting the mesothelin polypeptide with an ADC comprising (or consisting essentially of, or consisting of) an antigen binging domain covalently linked to a drug, wherein the antigen binging domain comprises an antibody of the first aspect described above, an antigen-binding fragment of the second aspect described above, or an antibody domain of the third aspect described above. The antigen binding domain can comprise a scFv having the ability to bind to a mesothelin polypeptide. The antigen binding domain can comprise a VH domain having the ability to bind to a mesothelin polypeptide. The drug can be selected from the group consisting of auristatins, mertansine, or pyrrolobenzodiazepine (PBD) dimers. The contacting can be performed in vitro. The contacting can be performed in vivo. The contacting can be performed within a mammal by administering the chimeric antigen receptor, the cell engager, or the ADC to the mammal. The mammal can be a human.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts amino acid residues 1 to 630 of a human mesothelin polypeptide (SEQ ID NO:97). The underlined amino acid sequence (residues 296 to 606) of this human mesothelin polypeptide depicts a fragment (SEQ ID NO:531).

FIG. 2 depicts the amino acid sequence of a VH domain designated Clone #1 (ab1). The CDRs and framework sequences also are delineated.

FIG. 3 depicts the amino acid sequence of a VH domain designated Clone #2 (ab2). The CDRs and framework sequences also are delineated.

FIG. 4 depicts the amino acid sequence of a VH domain designated Clone #3 (ab3). The CDRs and framework sequences also are delineated.

FIG. 5 depicts the amino acid sequence of a VH domain designated Clone #4 (ab4). The CDRs and framework sequences also are delineated.

FIG. 6 depicts the amino acid sequence of a VH domain designated Clone #5 (ab5). The CDRs and framework sequences also are delineated.

FIG. 7 depicts the amino acid sequence of a VH domain designated Clone #6 (ab6). The CDRs and framework sequences also are delineated.

FIG. 8 depicts the amino acid sequence of a VH domain designated Clone #7 (ab7). The CDRs and framework sequences also are delineated.

FIG. 9 depicts the amino acid sequence of a VH domain designated Clone #8 (ab8). The CDRs and framework sequences also are delineated.

FIG. 10 depicts the amino acid sequence of a VH domain designated Clone #9 (ab9). The CDRs and framework sequences also are delineated.

FIG. 11 depicts the amino acid sequence of a VH domain designated Clone #10 (ab10). The CDRs and framework sequences also are delineated.

FIG. 12 depicts the amino acid sequence of a VH domain designated Clone #11 (ab11). The CDRs and framework sequences also are delineated.

FIG. 13 depicts the amino acid sequence of a VH domain designated Clone #12 (ab12). The CDRs and framework sequences also are delineated.

FIG. 14 depicts the nucleic acid sequences encoding the indicated domains of Clones #1-#12.

FIG. 15 depicts exemplary linker amino acid sequences that can be used for scFv's, CARs, or cell engagers.

FIG. 16 depicts the amino acid sequences of exemplary hinges that can be used to design a CAR.

FIG. 17 depicts the amino acid sequences of exemplary transmembrane domains that can be used to design a CAR.

FIG. 18 depicts the amino acid sequences of exemplary intracellular signaling domains that can be used to design a CAR.

FIG. 19 depicts the amino acid sequences of exemplary antigen binding domains that can be used to design cell engagers that bind to T cells.

FIG. 20 depicts the amino acid sequences of exemplary antigen binding domains that can be used to design cell engagers that bind to NK cells.

FIG. 21 is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a binder provided herein (e.g., a binder that includes a set of three CDRs such as CDR1, CDR2, and CDR3 of a VH domain provided herein, for example, SEQ ID NOs:1-3; SEQ ID NOs:9-11; SEQ ID NOs:17-19; SEQ ID NOs:25-27; SEQ ID NOs:33-SEQ ID NOs:41-43; SEQ ID NOs:49-51; SEQ ID NOs:57-59; SEQ ID NOs:65-67; SEQ ID NOs:73-75; SEQ ID NOs:81-83; or SEQ ID NOs:89-91), followed by a CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 22 depicts an amino acid of a CAR (CAR #1) designed to include a binder created using the three CDRs of the Clone #1 VH domain and a nucleic acid sequence encoding that CAR. The VH domain sequence (SEQ ID NO:8) of Clone #1 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 23 depicts an amino acid of a CAR (CAR #2) designed to include a binder created using the three CDRs of the Clone #2 VH domain and a nucleic acid sequence encoding that CAR. The VH domain sequence (SEQ ID NO:16) of Clone #2 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 24 depicts an amino acid of a CAR (CAR #3) designed to include a binder created using the three CDRs of the Clone #3 VH domain and a nucleic acid sequence encoding that CAR. The VH domain sequence (SEQ ID NO:24) of Clone #3 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 25 depicts an amino acid of a CAR (CAR #4) designed to include a binder created using the three CDRs of the Clone #4 VH domain and a nucleic acid sequence encoding that CAR. The VH domain sequence (SEQ ID NO:32) of Clone #4 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 26 depicts an amino acid of a CAR (CAR #5) designed to include a binder created using the three CDRs of the Clone #5 VH domain and a nucleic acid sequence encoding that CAR. The VH domain sequence (SEQ ID NO:40) of Clone #5 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 27 depicts an amino acid of a CAR (CAR #6) designed to include a binder created using the three CDRs of the Clone #6 VH domain and a nucleic acid sequence encoding that CAR. The VH domain sequence (SEQ ID NO:48) of Clone #6 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 28 depicts an amino acid of a CAR (CAR #7) designed to include a binder created using the three CDRs of the Clone #7 VH domain and a nucleic acid sequence encoding that CAR. The VH domain sequence (SEQ ID NO:56) of Clone #7 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 29 depicts an amino acid of a CAR (CAR #8) designed to include a binder created using the three CDRs of the Clone #8 VH domain and a nucleic acid sequence encoding that CAR. The VH domain sequence (SEQ ID NO:64) of Clone #8 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 30 depicts an amino acid of a CAR (CAR #9) designed to include a binder created using the three CDRs of the Clone #9 VH domain and a nucleic acid sequence encoding that CAR. The VH domain sequence (SEQ ID NO:72) of Clone #9 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 31 depicts an amino acid of a CAR (CAR #10) designed to include a binder created using the three CDRs of the Clone #10 VH domain and a nucleic acid sequence encoding that CAR. The VH domain sequence (SEQ ID NO:80) of Clone #10 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 32 depicts an amino acid of a CAR (CAR #11) designed to include a binder created using the three CDRs of the Clone #11 VH domain and a nucleic acid sequence encoding that CAR. The VH domain sequence (SEQ ID NO:88) of Clone #11 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 33 depicts an amino acid of a CAR (CAR #12) designed to include a binder created using the three CDRs of the Clone #12 VH domain and a nucleic acid sequence encoding that CAR. The VH domain sequence (SEQ ID NO:96) of Clone #12 is followed CD8 hinge sequence (e.g., SEQ ID NO:533), followed by a CD8 transmembrane sequence (e.g., SEQ ID NO:534), followed by a human 4-1BB (CD137) intracellular signaling domain sequence (e.g., SEQ ID NO:141), followed by a human CD3ζ intracellular signaling domain sequence (e.g., SEQ ID NO:140).

FIG. 34 is a graph plotting the percent lysis of target cells (AsPC1) by effector cells (T cells transfected with CAR #4 or CAR #12). Untransduced T cells (Mock) were used as control effector cells.

FIG. 35 is a graph plotting the percent lysis of target cells (NCI H2452) by effector cells (T cells transfected with CAR #4). Untransduced T cells (Mock) were used as control effector cells.

### DETAILED DESCRIPTION

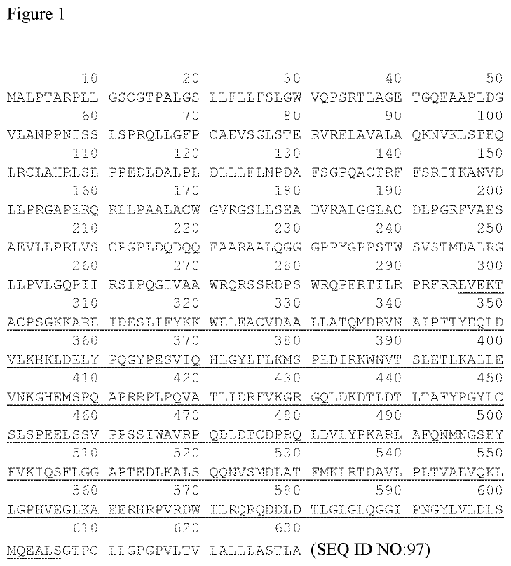

This document provides binders (e.g., antibodies, antigen binding fragments, antibody domains, CARs, cell engagers, and ADCs) that bind (e.g., specifically bind) to a mesothelin polypeptide (e.g., a human mesothelin polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:97 or SEQ ID NO:531 (see, e.g., FIG. 1).

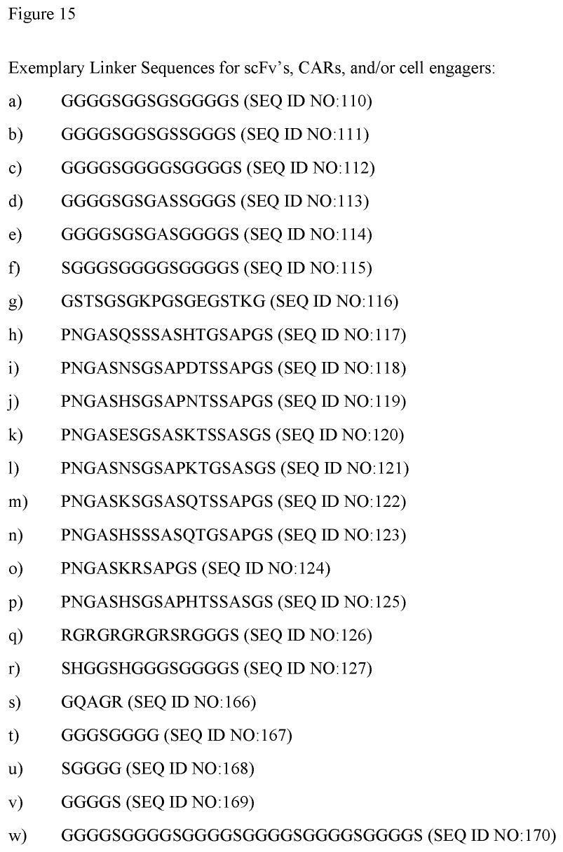

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Farber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length or a polypeptide linker as set forth in FIG. 15). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 15). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains.

An antibody provided herein can include the CDRs as described herein (e.g., as described in Table 37) and can be configured to be a human antibody, a humanized antibody, or a chimeric antibody. In some cases, an antibody provided herein can include the CDRs as described herein (e.g., as described in Table 37) and can be a monoclonal antibody. In some cases, an antibody provided herein can include the CDRs as described herein (e.g., as described in Table 37) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 37) and can be configured to be a human antigen binding fragment, a humanized antigen binding fragment, or a chimeric antigen binding fragment. In some cases, an antigen binding fragment provided herein can include the CDRs as described herein (e.g., as described in Table 37) and can be a monoclonal antigen binding fragment. In some cases, an antigen binding fragment provided herein can include the CDRs as described herein (e.g., as described in Table 37) and can be configured as an Fab antibody.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 37) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 37) and can be a monoclonal antibody domain. In some cases, an antibody domain provided herein can include the CDRs as described herein (e.g., as described in Table 37) and can be engineered as a single VH domain or a single VL domain.

An anti-mesothelin antibody, anti-mesothelin antigen binding fragment, or anti-mesothelin antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-mesothelin antibody) can be a scFv antibody. In some cases, an antigen binding fragment provided herein (e.g., an anti-mesothelin antibody fragment) can be an Fab. In some cases, an antibody provided herein (e.g., an anti-mesothelin antibody) can be a fully intact antibody consisting of both VH and VL. In some cases, an antibody domain provided herein (e.g., an anti-mesothelin antibody domain) can be a VH domain.

The term “chimeric antigen receptor” as used herein refers to a chimeric polypeptide that is designed to include an antigen binding domain, an optional hinge, a transmembrane domain, and one or more intracellular signaling domains. As described herein, the antigen binding domain of a CAR provided herein can be designed to bind to a mesothelin polypeptide (e.g., a human mesothelin polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a mesothelin polypeptide (e.g., a human mesothelin polypeptide). In some examples, a CAR provided herein can be designed to include an antigen binding domain that includes a single set of three CDRs (e.g., a CDR1, CDR2, and CDR3) of an antibody domain (e.g., a VH domain) provided herein (e.g., SEQ ID NOs:1-3; SEQ ID NOs:9-11; SEQ ID NOs:17-19; SEQ ID NOs:25-27; SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; or SEQ ID NOs:89-91). In some cases, an antigen binding domain of a CAR targeting a mesothelin polypeptide can be designed to include a VH domain described herein or a scFv antibody described herein.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as hinge to make a CAR described herein. For example, any one of the linker sequences set forth in FIG. 15 can be used as a hinge of a CAR described herein.

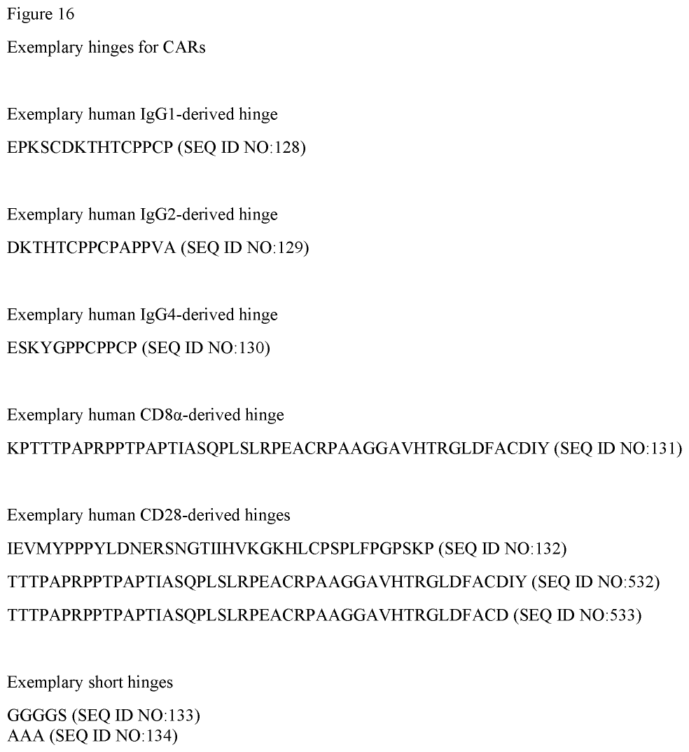

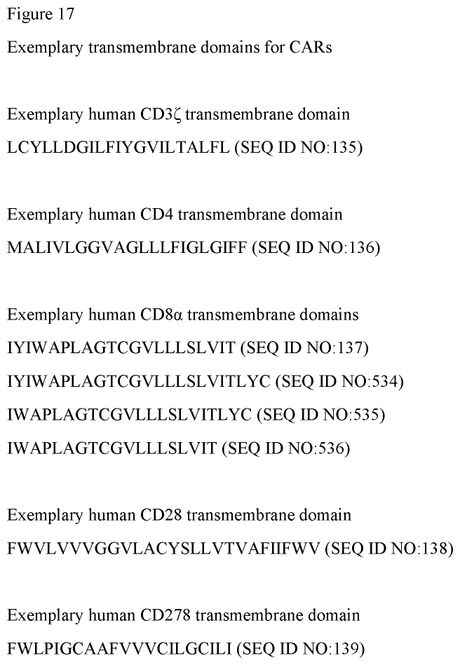

In some cases, a CAR provided herein can be designed to include a hinge that comprises, consists essentially of, or consists of one of the amino acid sequences set forth in FIG. 16 or FIG. 15. In some cases, a CAR provided herein can be designed to include a hinge that comprises, consists essentially of, or consists of one of the amino acid sequences set forth in FIG. 16 or FIG. 15 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in FIG. 16 or FIG. 17 with two or less, three or less, four or less, five or less, six or less, seven or less, eight or less, nine or less, or ten or less amino acid deletions, additions, substitutions, or combinations thereof.

A CAR provided herein can be designed to include any appropriate transmembrane domain. For example, the transmembrane domain of a CAR provided herein can be, without limitation, a CD3ζ transmembrane domain, a CD4 transmembrane domain, a CD8α transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in FIG. 17. In some cases, a CAR provided herein can be designed to include a transmembrane domain that comprises, consists essentially of, or consists of one of the amino acid sequences set forth in FIG. 17 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in FIG. 17 with two or less, three or less, four or less, five or less, six or less, seven or less, eight or less, nine or less, or ten or less amino acid deletions, additions, substitutions, or combinations thereof.

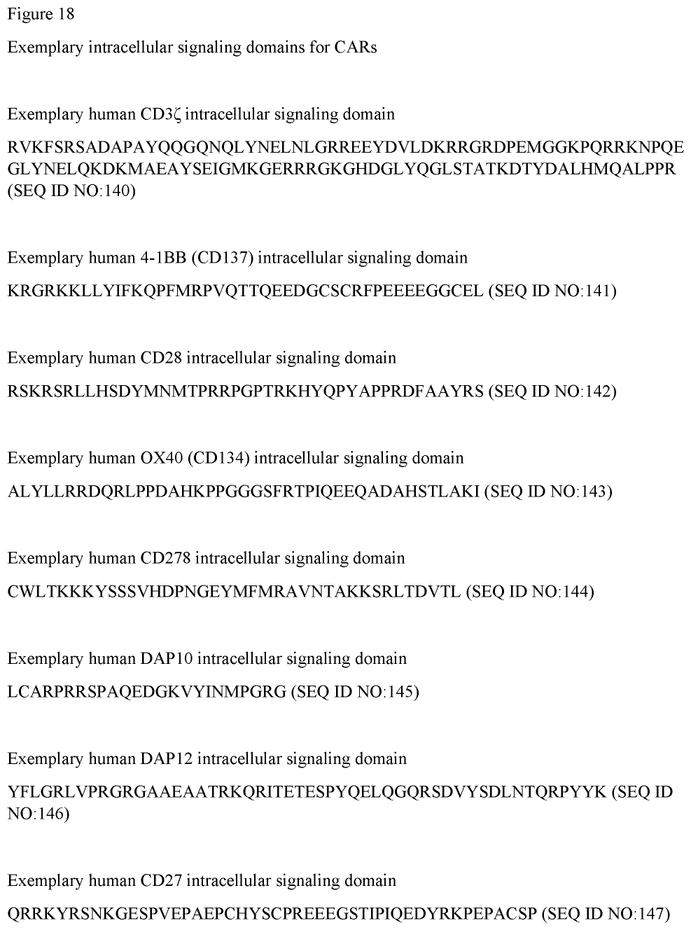

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in FIG. 18. In some cases, a CAR provided herein can be designed to include at least one intracellular signaling domain that comprises, consists essentially of, or consists of one of the amino acid sequences set forth in FIG. 18 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in FIG. 18 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting a mesothelin polypeptide can be designed to include a VH domain comprising SEQ ID NO:1, SEQ ID NO:2, and SEQ ID NO:3, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:1, SEQ ID NO:2, and SEQ ID NO:3, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:8, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:8, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:9, SEQ ID NO:10, and SEQ ID NO:11, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:9, SEQ ID NO:10, and SEQ ID NO:11, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:16, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:16, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:17, SEQ ID NO:18, and SEQ ID NO:19, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:17, SEQ ID NO:18, and SEQ ID NO:19, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:24, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:24, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:25, SEQ ID NO:26, and SEQ ID NO:27, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:25, SEQ ID NO:26, and SEQ ID NO:27, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:32, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:32, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:33, SEQ ID NO:34, and SEQ ID NO:35, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:33, SEQ ID NO:34, and SEQ ID NO:35, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:40, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:40, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:41, SEQ ID NO:42, and SEQ ID NO:43, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:41, SEQ ID NO:42, and SEQ ID NO:43, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:48, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:48, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:49, SEQ ID NO:50, and SEQ ID NO:51, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:49, SEQ ID NO:50, and SEQ ID NO:51, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:56, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:56, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:57, SEQ ID NO:58, and SEQ ID NO:59, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:57, SEQ ID NO:58, and SEQ ID NO:59, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:64, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:64, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:65, SEQ ID NO:66, and SEQ ID NO:67, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:65, SEQ ID NO:66, and SEQ ID NO:67, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:72, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:72, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:73, SEQ ID NO:74, and SEQ ID NO:75, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:73, SEQ ID NO:74, and SEQ ID NO:75, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:80, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:80, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:81, SEQ ID NO:82, and SEQ ID NO:83, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:81, SEQ ID NO:82, and SEQ ID NO:83, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:88, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:88, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:89, SEQ ID NO:90, and SEQ ID NO:91, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:89, SEQ ID NO:90, and SEQ ID NO:91, followed by SEQ ID NO:131, followed by SEQ ID NO:137, followed by SEQ ID NO:141, followed by SEQ ID NO:140.

In some cases, a CAR targeting a mesothelin polypeptide can be designed to include a VH domain comprising SEQ ID NO:96, followed by a hinge such as a hinge/linker set forth in FIG. 15 or FIG. 16 (e.g., a human CD8α hinge), followed by a transmembrane domain such as a transmembrane domain set forth in FIG. 17 (e.g., a human CD8α transmembrane domain), followed by one or more intracellular signaling domains such as one or more intracellular signaling domain set forth in FIG. 18 (e.g., a human 4-1BB intracellular signaling domain followed by a human CD3ζ intracellular signaling domain). For example, a CAR targeting a mesothelin polypeptide can be designed to include a VH domain comprising SEQ ID NO:96, followed by SEQ ID NO:131, followed by SEQ ID NO:137, followed by SEQ ID NO:141, followed by SEQ ID NO:140.

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a mesothelin polypeptide (e.g., a human mesothelin polypeptide) and at least one antigen binding domain having the ability to bind to an antigen expressed on the surface of a cell (e.g., a T cell or an NK cell). In some cases, a cell engager described herein can link a mesothelin⁺ cell (e.g., a mesothelin⁺ cancer cell) to another cell (e.g., a T cell or an NK cell) via the two or more antigen binding domains of the cell engager.

When a cell engager includes an antigen binding domain having the ability to bind to a mesothelin polypeptide (e.g., a human mesothelin polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to a mesothelin polypeptide (e.g., a human mesothelin polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to a mesothelin polypeptide (e.g., a human mesothelin polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a mesothelin polypeptide (e.g., a human mesothelin polypeptide). In some examples, a cell engager provided herein can be designed to include an antigen binding domain that includes a single set of three CDRs (e.g., a CDR1, CDR2, and CDR3) of an antibody domain (e.g., a VH domain) provided herein (e.g., SEQ ID NOs:1-3; SEQ ID NOs:9-11; SEQ ID NOs:17-19; SEQ ID NOs:25-27; SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; or SEQ ID NOs:89-91).

In some cases, an antigen binding domain of a cell engager targeting a mesothelin polypeptide can be designed to include a VH domain described herein or a scFv/Fab antibody described herein. In some cases, an antigen binding domain of a CAR described herein that has the ability to bind to a mesothelin polypeptide (e.g., a human mesothelin polypeptide) can be used as an antigen binding domain of a cell engager that targets mesothelin⁺ cells.

As described herein, a cell engager can be designed to include at least one antigen binding domain having the ability to bind to a mesothelin polypeptide (e.g., a human mesothelin polypeptide) and at least one other antigen binding domain. That at least one other antigen binding domain can have the ability to bind to any appropriate antigen expressed on the surface of a cell. For example, when designing a cell engager such as a BiTE to link a mesothelin⁺ cell and a T cell, the cell engager can include an antigen binding domain having the ability to bind to a mesothelin polypeptide (e.g., a human mesothelin polypeptide) and an antigen binding domain having the ability to bind to a polypeptide expressed on the surface of a T cell. Examples example of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

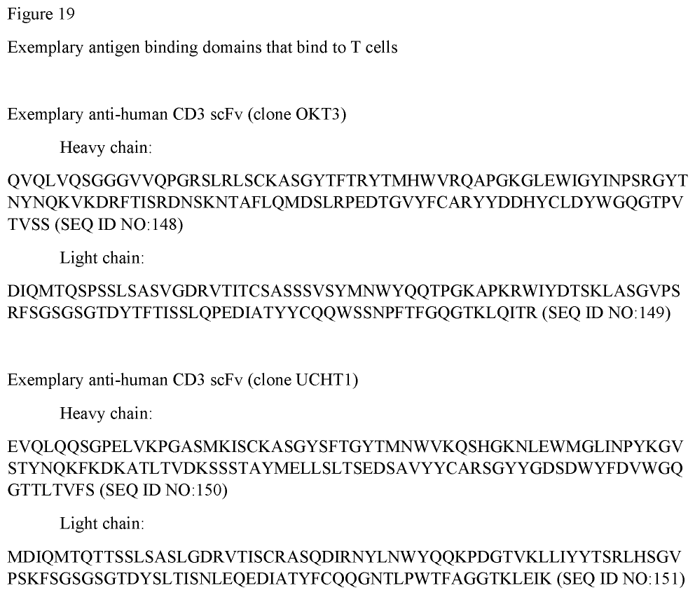

In some cases, a cell engager provided herein can be designed to include an antigen binding domain that comprises, consists essentially of, or consists of one of the amino acid sequences set forth in FIG. 19. In some cases, a cell engager provided herein can be designed to include an antigen binding domain that comprises, consists essentially of, or consists of one of the amino acid sequences set forth in FIG. 19 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in FIG. 19 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link a mesothelin⁺ cell and an NK cell, the cell engager can include an antigen binding domain having the ability to bind to a mesothelin polypeptide (e.g., a human mesothelin polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

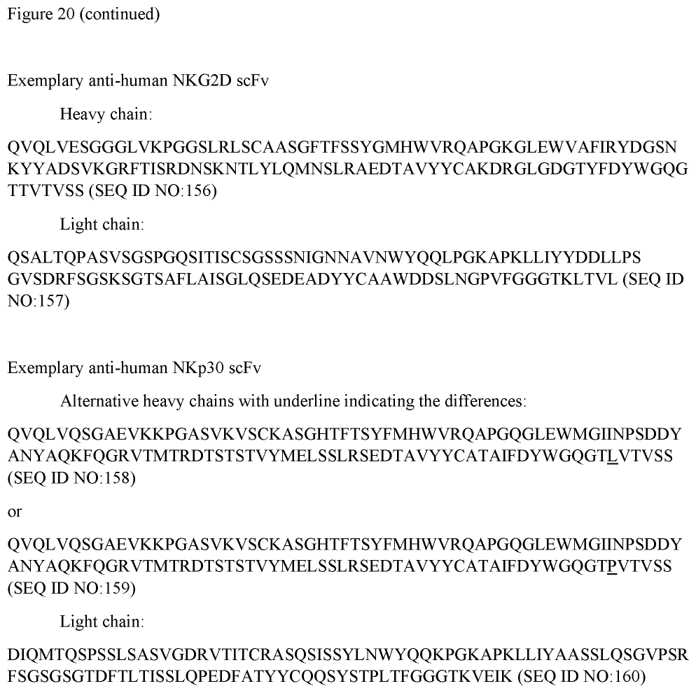

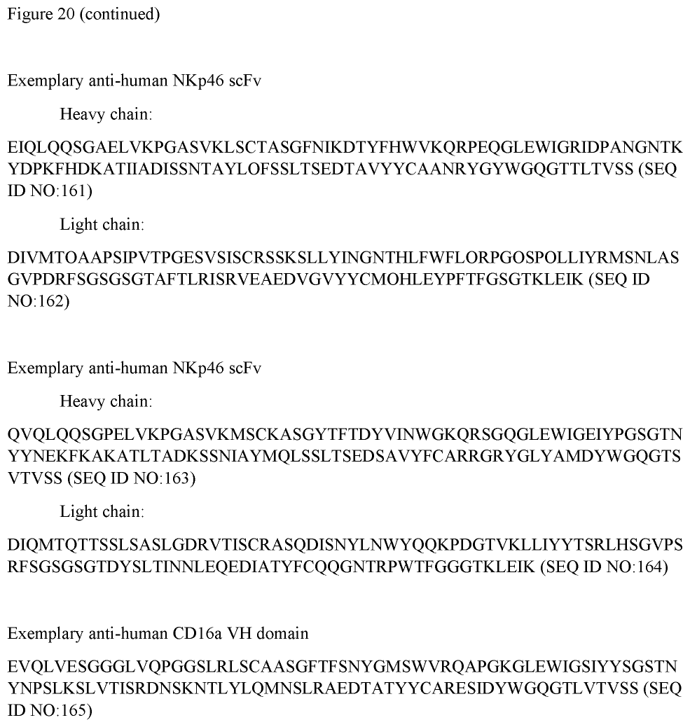

In some cases, a cell engager provided herein can be designed to include an antigen binding domain (e.g., a scFv or VH) that comprises, consists essentially of, or consists of one or more of the amino acid sequences set forth in FIG. 20. In some cases, a cell engager provided herein can be designed to include an antigen binding domain (e.g., a scFv or VH) that comprises, consists essentially of, or consists of one of the amino acid sequences set forth in FIG. 20 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in FIG. 20 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in FIG. 15. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:112) linker. In some cases, a hinge of a CAR described herein can be used as a linker to make a cell engager described herein. For example, any one of the sequences set forth in FIG. 16 can be used as a linker of a cell engager described herein.

In some cases, a cell engager provided herein can be designed to include a linker that comprises, consists essentially of, or consists of one of the amino acid sequences set forth in FIG. 15 or FIG. 16. In some cases, a cell engager provided herein can be designed to include a linker that comprises, consists essentially of, or consists of one of the amino acid sequences set forth in FIG. 15 or FIG. 16 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in FIG. 15 or FIG. 16 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting a mesothelin polypeptide can be designed to include a VH domain comprising SEQ ID NO:1, SEQ ID NO:2, and SEQ ID NO:3, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:8, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:1, SEQ ID NO:2, and SEQ ID NO:3, followed by a linker such as a linker/hinge set forth in Figure or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:8, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:9, SEQ ID NO:10, and SEQ ID NO:11, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:16, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:9, SEQ ID NO:10, and SEQ ID NO:11, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:16, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:17, SEQ ID NO:18, and SEQ ID NO:19, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:24, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:17, SEQ ID NO:18, and SEQ ID NO:19, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:24, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:25, SEQ ID NO:26, and SEQ ID NO:27, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:32, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:25, SEQ ID NO:26, and SEQ ID NO:27, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:32, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:33, SEQ ID NO:34, and SEQ ID NO:35, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:40, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:33, SEQ ID NO:34, and SEQ ID NO:35, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:40, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:41, SEQ ID NO:42, and SEQ ID NO:43, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:48, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:41, SEQ ID NO:42, and SEQ ID NO:43, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:48, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:49, SEQ ID NO:50, and SEQ ID NO:51, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:56, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:49, SEQ ID NO:50, and SEQ ID NO:51, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:56, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:57, SEQ ID NO:58, and SEQ ID NO:59, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:64, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:57, SEQ ID NO:58, and SEQ ID NO:59, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:64, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:65, SEQ ID NO:66, and SEQ ID NO:67, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:72, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:65, SEQ ID NO:66, and SEQ ID NO:67, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:72, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:73, SEQ ID NO:74, and SEQ ID NO:75, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:80, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:73, SEQ ID NO:74, and SEQ ID NO:75, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:80, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:81, SEQ ID NO:82, and SEQ ID NO:83, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:88, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:81, SEQ ID NO:82, and SEQ ID NO:83, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:88, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a mesothelin polypeptide can be designed to include a VH domain comprising SEQ ID NO:89, SEQ ID NO:90, and SEQ ID NO:91, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiTE) targeting a mesothelin polypeptide can be designed to include a VH domain comprising SEQ ID NO:96, followed by a linker such as a linker/hinge set forth in FIG. 15 or FIG. 16 (e.g., SEQ ID NO:112), followed by an antigen binding domain having the ability to bind to a polypeptide expressed on the surface of a T cell (e.g., an anti-human CD3 scFv).

In some cases, a cell engager (e.g., a BiKE or a TriKE) targeting a mesothelin polypeptide can be designed to include a VH domain comprising SEQ ID NO:89, SEQ ID NO:90, and SEQ ID NO:91, followed by a linker such as a linker/hinge set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a mesothelin polypeptide can be designed to include a VH domain comprising SEQ ID NO:96, followed by a linker such as a hinge/linker set forth in FIG. 15 or FIG. 16 (e.g., SEQ ID NO:112), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

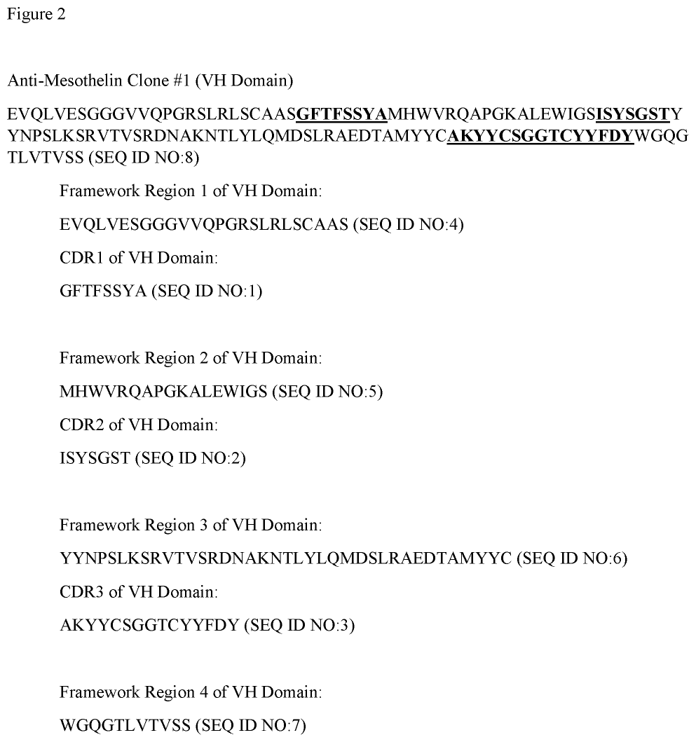

In one embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications). An example of such an antibody domain having these CDRs and the ability to bind to a mesothelin polypeptide (e.g., a human mesothelin polypeptide) includes, without limitation, the VH domain set forth in FIG. 2.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 2 can be designed to include framework regions as set forth in FIG. 2 or can be designed to include one or more framework regions from another antibody or antibody fragment. For example, an antibody domain (e.g., a VH domain) can be designed to include the three CDRs set forth in FIG. 2 and the framework regions set forth in FIG. 2 except that framework region 1 having the amino acid set forth in SEQ ID NO:4 is replaced with a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 2, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:8. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:8. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs:1, 2, and 3. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:1 include, without limitation, those set forth in Table 1.

TABLE 1
Exemplary CDR1s that consist essentially
of the amino acid sequence set forth in
SEQ ID NO: 1.

| Sequence | SEQ ID NO: |
|---|---|
| GFTFSDYY | 171 |
| GYTFTSYY | 172 |
| GYTFTGYY | 173 |
| GFTFSSYW | 174 |
| GFTFSNSD | 175 |
| GGTFSSYA | 176 |
| GFTFDDYA | 177 |
| GFTFSDYY | 178 |
| GFTFSDHY | 179 |
| GGSFSGYY | 180 |

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:2 include, without limitation, those set forth in Table 2.

TABLE 2
Exemplary CDR2s that consist essentially
of the amino acid sequence set forth in
SEQ ID NO: 2.

| Sequence | SEQ ID NO: |
|---|---|
| INPNSGGT | 181 |
| ISAYNGNT | 182 |
| ISSSGST | 183 |
| IGTAGDT | 184 |
| ISSSSSYI | 185 |
| ISGSGGST | 186 |
| ISYDGSNK | 187 |
| IYSGGST | 188 |
| IYHSGST | 189 |
| ISGSGGST | 190 |

As used herein, a “CDR3 that consists essentially of the amino acid sequence set forth in SEQ ID NO:3” is a CDR3 that has zero, one, or two amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:3 include, without limitation, those set forth in Table 3.

TABLE 3
Exemplary CDR3s that consist essentially
of the amino acid sequence set forth in
SEQ ID NO: 3.

| Sequence | SEQ ID NO: |
|---|---|
| ARYYCSGGTCYYFDY | 191 |
| AAYYCSGGTCYYFDY | 192 |
| AKDYYCSGGTCYYFDY | 193 |
| VRYYCSGGTCYYFDY | 194 |
| SRYYCSGGTCYYFDY | 195 |
| AKYYCSGGTCYYFDY | 196 |
| KKYYCSGGTCYYFDY | 197 |
| AKDYYCSGGTCYYFDY | 198 |
| ARIYYCSGGTCYYFDY | 199 |
| ATYYCSGGTCYYFDY | 200 |

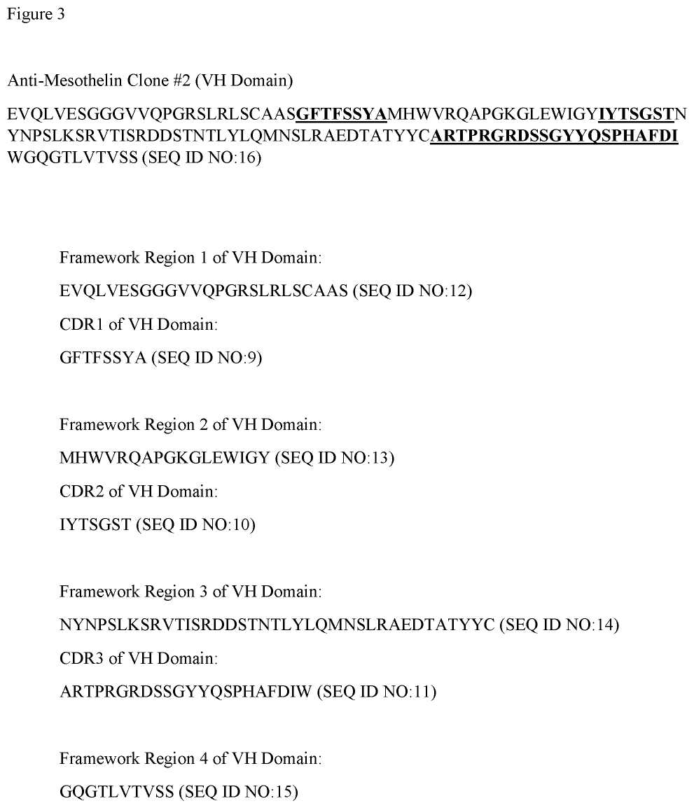

In one embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth in SEQ ID NO:11 (or a variant of SEQ ID NO:11 with one or two amino acid modifications). An example of such an antibody domain having these CDRs and the ability to bind to a mesothelin polypeptide (e.g., a human mesothelin polypeptide) includes, without limitation, the VH domain set forth in FIG. 3.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth in SEQ ID NO:11 (or a variant of SEQ ID NO:11 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:12 (or a variant of SEQ ID NO:12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 3 can be designed to include framework regions as set forth in FIG. 3 or can be designed to include one or more framework regions from another antibody or antibody fragment. For example, an antibody domain (e.g., a VH domain) can be designed to include the three CDRs set forth in FIG. 3 and the framework regions set forth in FIG. 3 except that framework region 1 having the amino acid set forth in SEQ ID NO:12 is replaced with a framework region 1 having the amino acid set forth in SEQ ID NO:4, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 3, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:16, provided that the heavy chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:16, provided that the heavy chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 9, 10, and 11.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:9 include, without limitation, those set forth in Table 4.

TABLE 4
Exemplary CDRIs that consist essentially
of the amino acid sequence set forth in
SEQ ID NO: 9.

| Sequence | SEQ ID NO: |
|---|---|
| GYTFTGYY | 201 |
| GYTFTSYA | 202 |
| GYTFTSYG | 203 |
| GFTFTSSA | 204 |
| GFTFSSYW | 205 |
| GFTFDDYA | 206 |
| GFTFSSYD | 207 |
| GFTFSSYG | 208 |
| GFTFSSSA | 209 |
| GFTFSDHY | 210 |

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:10 include, without limitation, those set forth in Table 5.

TABLE 5
Exemplary CDR2s that consist essentially
of the amino acid sequence set forth in
SEQ ID NO: 10.

| Sequence | SEQ ID NO: |
|---|---|
| INPNSGGT | 211 |
| ISAYNGNT | 212 |
| ISSSGST | 213 |
| IGTAGDT | 214 |
| ISSSSSYI | 215 |
| ISGSGGST | 216 |
| ISYDGSNK | 217 |
| IYSGGST | 218 |
| IYHSGST | 219 |
| ISGSGGST | 220 |

As used herein, a “CDR3 that consists essentially of the amino acid sequence set forth in SEQ ID NO:11” is a CDR3 that has zero, one, or two amino acid substitutions within SEQ ID NO:11, that has zero, one, two, three, four, or five amino acid residues directly preceding SEQ ID NO:11, and/or that has zero, one, two, three, four, or five amino acid residues directly following SEQ ID NO:11, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:11 include, without limitation, those set forth in Table 6.

TABLE 6
Exemplary CDR3s that consist essentially
of the amino acid sequence set forth in
SEQ ID NO: 11.

| Sequence | SEQ ID NO: |
|---|---|
| ARRTPRGRDSSGYYQSPHAFDI | 221 |
| ATTPRGRDSSGYYQSPHAFDI | 222 |
| AATPRGRDSSGYYQSPHAFDI | 223 |
| VRTPRGRDSSGYYQSPHAFDI | 224 |
| AHRTPRGRDSSGYYQSPHAFDI | 225 |
| ARITPRGRDSSGYYQSPHAFDI | 226 |
| AKDTPRGRDSSGYYQSPHAFDI | 227 |
| TTTPRGRDSSGYYQSPHAFDI | 228 |
| SRTPRGRDSSGYYQSPHAFDI | 229 |
| AKTPRGRDSSGYYQSPHAFDI | 230 |

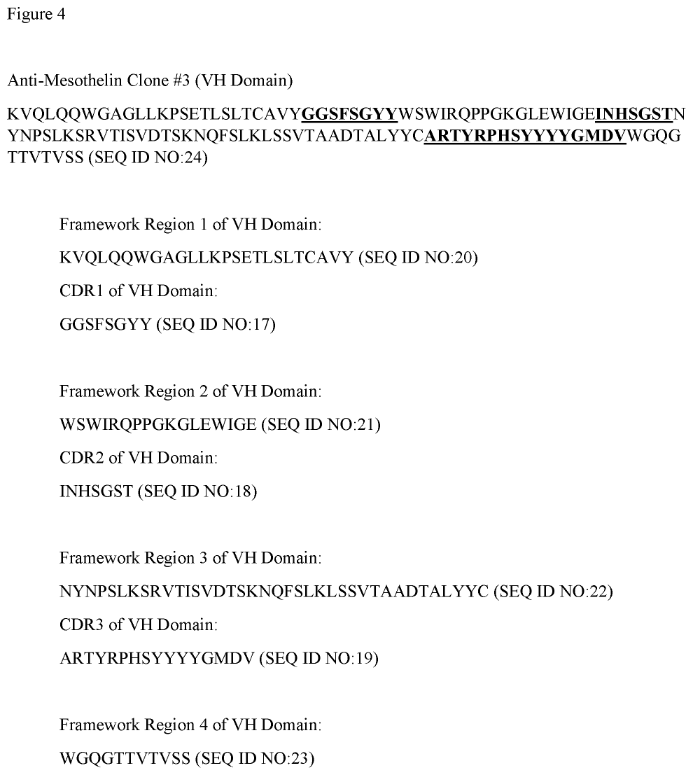

In one embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications). An example of such an antibody domain having these CDRs and the ability to bind to a mesothelin polypeptide (e.g., a human mesothelin polypeptide) includes, without limitation, the VH domain set forth in FIG. 4.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 4 can be designed to include framework regions as set forth in FIG. 4 or can be designed to include one or more framework regions from another antibody or antibody fragment. For example, an antibody domain (e.g., a VH domain) can be designed to include the three CDRs set forth in FIG. 4 and the framework regions set forth in FIG. 4 except that framework region 1 having the amino acid set forth in SEQ ID NO:20 is replaced with a framework region 1 having the amino acid set forth in SEQ ID NO:4, a framework region 1 having the amino acid set forth in SEQ ID NO:12, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 4, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:24. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:24. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs:17, 18, and 19. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs:17, 18, and 19.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:17, 18, and 19.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO:18, and (iii) a CDR3 that comprises, consists essentially of, or consists of the amino acid sequence set forth in SEQ ID NO:19. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:17 include, without limitation, those set forth in Table 7.

TABLE 7
Exemplary CDR1s that consist essentially of
the amino acid sequence set forth
in SEQ ID NO: 17.

| Sequence | SEQ ID NO: |
|---|---|
| GYTFTGYY | 231 |
| GYTFTSYA | 232 |
| GYTFTSYG | 233 |
| GFTFTSSA | 234 |
| GFTFSSYW | 235 |
| GFTFDDYA | 236 |
| GFTFSSYA | 237 |
| GFTFSSYG | 238 |
| GFTFSSSA | 239 |
| GFTFSDHY | 240 |

As used herein, a “CDR2 that consists essentially of the amino acid sequence set forth in SEQ ID NO:18” is a CDR2 that has zero, one, or two amino acid substitutions within SEQ ID NO:18, that has zero, one, two, three, four, or five amino acid residues directly preceding SEQ ID NO:18, and/or that has zero, one, two, three, four, or five amino acid residues directly following SEQ ID NO:18, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:18 include, without limitation, those set forth in Table 8.

TABLE 8
Exemplary CDR2s that consist essentially of
the amino acid sequence set forth
in SEQ ID NO: 18.

| Sequence | SEQ ID NO: |
|---|---|
| INPNSGGT | 241 |
| ISAYNGNT | 242 |
| ISSSGST | 243 |
| IGTAGDT | 244 |
| ISSSSSYI | 245 |
| ISGSGGST | 246 |
| ISYDGSNK | 247 |
| IYSGGST | 248 |
| IYHSGST | 249 |
| ISGSGGST | 250 |

As used herein, a “CDR3 that consists essentially of the amino acid sequence set forth in SEQ ID NO:19” is a CDR3 that has zero, one, or two amino acid substitutions within SEQ ID NO:19, that has zero, one, two, three, four, or five amino acid residues directly preceding SEQ ID NO:19, and/or that has zero, one, two, three, four, or five amino acid residues directly following SEQ ID NO:19, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:19 include, without limitation, those set forth in Table 9.

TABLE 9
Exemplary CDR3s that consist essentially
of the amino acid sequence set forth
in SEQ ID NO: 19.

| Sequence | SEQ ID NO: |
|---|---|
| ARRTYRPHSYYYYGMDV | 251 |
| ATTYRPHSYYYYGMDV | 252 |
| AATYRPHSYYYYGMDV | 253 |
| VRTYRPHSYYYYGMDV | 254 |
| AHRTYRPHSYYYYGMDV | 255 |
| ARITYRPHSYYYYGMDV | 256 |
| AKDTYRPHSYYYYGMDV | 257 |
| TTTYRPHSYYYYGMDV | 258 |
| SRTYRPHSYYYYGMDV | 259 |
| AKTYRPHSYYYYGMDV | 260 |

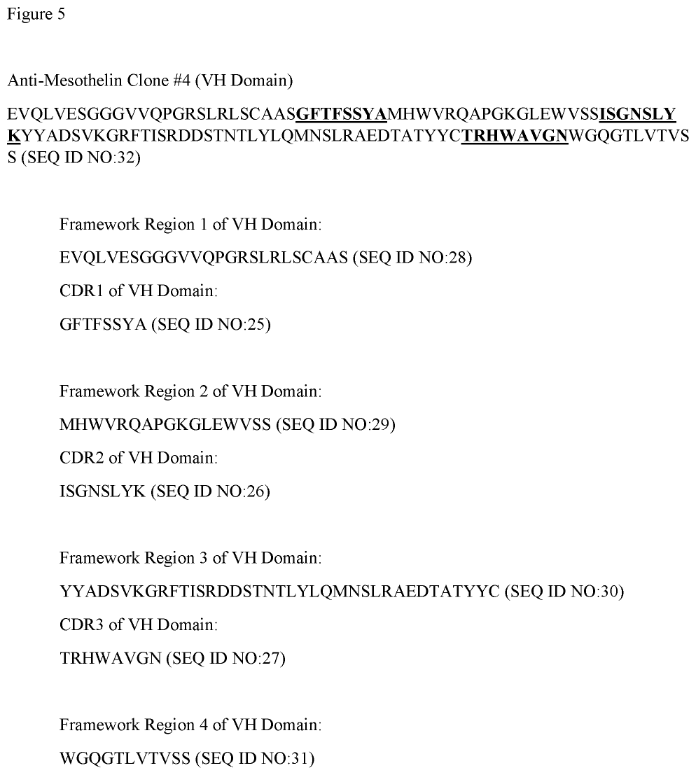

In one embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth in SEQ ID NO:27 (or a variant of SEQ ID NO:27 with one or two amino acid modifications). An example of such an antibody domain having these CDRs and the ability to bind to a mesothelin polypeptide (e.g., a human mesothelin polypeptide) includes, without limitation, the VH domain set forth in FIG. 5.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth in SEQ ID NO:27 (or a variant of SEQ ID NO:27 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO:29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO:31 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 5 can be designed to include framework regions as set forth in FIG. 5 or can be designed to include one or more framework regions from another antibody or antibody fragment. For example, an antibody domain (e.g., a VH domain) can be designed to include the three CDRs set forth in FIG. 5 and the framework regions set forth in Figure except that framework region 1 having the amino acid set forth in SEQ ID NO:28 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 5, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:32, provided that the heavy chain variable domain includes the amino acid sequences set forth in SEQ ID NOs:25, 26, and 27. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:32, provided that the heavy chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:25 include, without limitation, those set forth in Table 10.

TABLE 10
Exemplary CDR1s that consist essentially of
the amino acid sequence set forth
in SEQ ID NO: 25.

| Sequence | SEQ ID NO: |
|---|---|
| GYTFTGYY | 261 |
| GYTFTSYA | 262 |
| GYTFTSYG | 263 |
| GFTFTSSA | 264 |
| GFTFSSYW | 265 |
| GFTFDDYA | 266 |
| GFTFSSYD | 267 |
| GFTFSSYG | 268 |
| GFTFSSSA | 269 |
| GFTFSDHY | 270 |

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:26 include, without limitation, those set forth in Table 11.

TABLE 11
Exemplary CDR2s that consist essentially of
the amino acid sequence set forth
in SEQ ID NO: 26.

| Sequence | SEQ ID NO: |
|---|---|
| INPNSGGT | 271 |
| ISAYNGNT | 272 |
| ISSSGST | 273 |
| IGTAGDT | 274 |
| ISSSSSYI | 275 |
| ISGSGGST | 276 |
| ISYDGSNK | 277 |
| IYSGGST | 278 |
| IYHSGST | 279 |
| ISGSGGST | 280 |

As used herein, a “CDR3 that consists essentially of the amino acid sequence set forth in SEQ ID NO:27” is a CDR3 that has zero, one, or two amino acid substitutions within SEQ ID NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:27 include, without limitation, those set forth in Table 12.

TABLE 12
Exemplary CDR3s that consist essentially of
the amino acid sequence set forth
in SEQ ID NO: 27.

| Sequence | SEQ ID NO: |
|---|---|
| ARRTRHWAVGN | 281 |
| ATTRHWAVGN | 282 |
| AATRHWAVGN | 283 |
| VRTRHWAVGN | 284 |
| AHRTRHWAVGN | 285 |
| ARITRHWAVGN | 286 |
| AKDTRHWAVGN | 287 |
| TTTRHWAVGN | 288 |
| SRTRHWAVGN | 289 |
| AKTRHWAVGN | 290 |

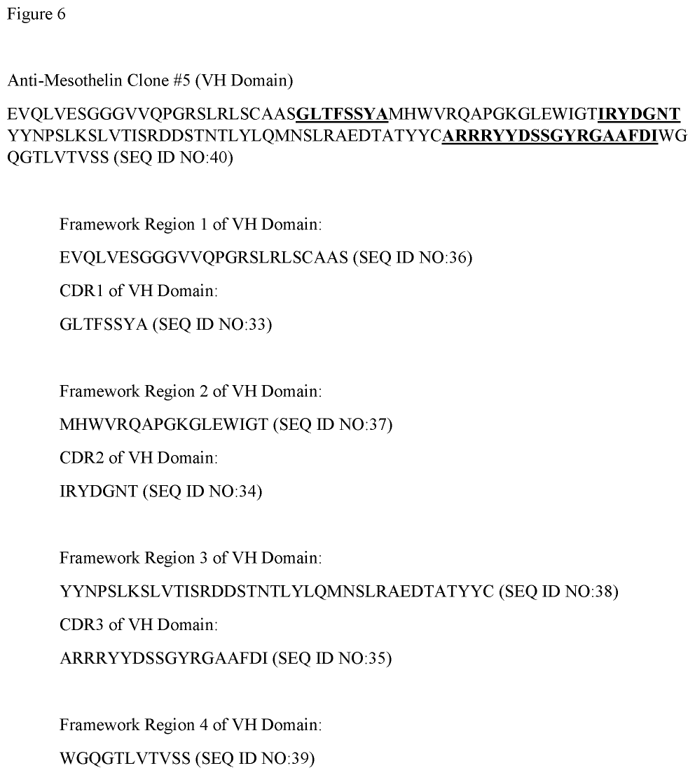

In one embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:33 (or a variant of SEQ ID NO:33 with one or two amino acid modifications), a CDR2 having the amino acid sequence set forth in SEQ ID NO:34 (or a variant of SEQ ID NO:34 with one or two amino acid modifications), and a CDR3 having the amino acid sequence set forth in SEQ ID NO:35 (or a variant of SEQ ID NO:35 with one or two amino acid modifications). An example of such an antibody domain having these CDRs and the ability to bind to a mesothelin polypeptide (e.g., a human mesothelin polypeptide) includes, without limitation, the VH domain set forth in FIG. 6.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:33 (or a variant of SEQ ID NO:33 with one or two amino acid modifications), a CDR2 having the amino acid sequence set forth in SEQ ID NO:34 (or a variant of SEQ ID NO:34 with one or two amino acid modifications), and a CDR3 having the amino acid sequence set forth in SEQ ID NO:35 (or a variant of SEQ ID NO:35 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:36 (or a variant of SEQ ID NO:36 with one, two, three, four, five, six, seven, eight, nine, ten, or more amino acid modifications), a framework region 2 having the amino acid sequence set forth in SEQ ID NO:37 (or a variant of SEQ ID NO:37 with one, two, three, four, five, six, seven, eight, nine, ten, or more amino acid modifications), a framework region 3 having the amino acid sequence set forth in SEQ ID NO:38 (or a variant of SEQ ID NO:38 with one, two, three, four, five, six, seven, eight, nine, ten, or more amino acid modifications), and a framework region 4 having the amino acid sequence set forth in SEQ ID NO:39 (or a variant of SEQ ID NO:39 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 6 can be designed to include framework regions as set forth in FIG. 6 or can be designed to include one or more framework regions from another antibody or antibody fragment. For example, an antibody domain (e.g., a VH domain) can be designed to include the three CDRs set forth in FIG. 6 and the framework regions set forth in FIG. 6 except that framework region 1 having the amino acid set forth in SEQ ID NO:36 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 6, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:40. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:40. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:40.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:40, provided that the heavy chain variable domain includes the amino acid sequences set forth in SEQ ID NOs:33, 34, and 35. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:40, provided that the heavy chain variable domain includes the amino acid sequences set forth in SEQ ID NOs:33, 34, and 35.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:40 or the amino acid set forth in SEQ ID NO:40 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:40 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:33, 34, and 35.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:33, (ii) a CDR2 that comprises, consists essentially of, or consists of the amino acid sequence set forth in SEQ ID NO:34, and (iii) a CDR3 that comprises, consists essentially of, or consists of the amino acid sequence set forth in SEQ ID NO:35. As used herein, a “CDR1 that consists essentially of the amino acid sequence set forth in SEQ ID NO:33” is a CDR1 that has zero, one, or two amino acid substitutions within SEQ ID NO:33, that has zero, one, two, three, four, or five amino acid residues directly preceding SEQ ID NO:33, and/or that has zero, one, two, three, four, or five amino acid residues directly following SEQ ID NO:33, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:33 include, without limitation, those set forth in Table 13.

TABLE 13
Exemplary CDR1s that consist essentially
of the amino acid sequence set forth
in SEQ ID NO: 33.

| Sequence | SEQ ID NO: |
|---|---|
| GYTFTGYY | 291 |
| GYTFTSYA | 292 |
| GYTFTSYG | 293 |
| GFTFTSSA | 294 |
| GFTFSSYW | 295 |
| GFTFDDYA | 296 |
| GFTFSSYA | 297 |
| GFTFSSYG | 298 |
| GFTFSSSA | 299 |
| GFTFSDHY | 300 |

As used herein, a “CDR2 that consists essentially of the amino acid sequence set forth in SEQ ID NO:34” is a CDR2 that has zero, one, or two amino acid substitutions within SEQ ID NO:34, that has zero, one, two, three, four, or five amino acid residues directly preceding SEQ ID NO:34, and/or that has zero, one, two, three, four, or five amino acid residues directly following SEQ ID NO:34, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:34 include, without limitation, those set forth in Table 14.

TABLE 14
Exemplary CDR2s that consist essentially of
the amino acid sequence set forth
in SEQ ID NO: 34.

| Sequence | SEQ ID NO: |
|---|---|
| INPNSGGT | 301 |
| ISAYNGNT | 302 |
| ISSSGST | 303 |
| IGTAGDT | 304 |
| ISSSSSYI | 305 |
| ISGSGGST | 306 |
| ISYDGSNK | 307 |
| IYSGGST | 308 |
| IYHSGST | 309 |
| ISGSGGST | 310 |

As used herein, a “CDR3 that consists essentially of the amino acid sequence set forth in SEQ ID NO:35” is a CDR3 that has zero, one, or two amino acid substitutions within SEQ ID NO:35, that has zero, one, two, three, four, or five amino acid residues directly preceding SEQ ID NO:35, and/or that has zero, one, two, three, four, or five amino acid residues directly following SEQ ID NO:35, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:35 include, without limitation, those set forth in Table 15.

TABLE 15
Exemplary CDR3s that consist essentially of
the amino acid sequence set forth
in SEQ ID NO: 35.

| Sequence | SEQ ID NO: |
|---|---|
| ARRRRYYDSSGYRGAAFDI | 311 |
| ATRRYYDSSGYRGAAFDI | 312 |
| AARRYYDSSGYRGAAFDI | 313 |
| VRRRYYDSSGYRGAAFDI | 314 |
| AHRRRYYDSSGYRGAAFDI | 315 |
| ARIRRYYDSSGYRGAAFDI | 316 |
| AKDRRYYDSSGYRGAAFDI | 317 |
| TTRRYYDSSGYRGAAFDI | 318 |
| SRRRYYDSSGYRGAAFDI | 319 |
| AKRRYYDSSGYRGAAFDI | 320 |

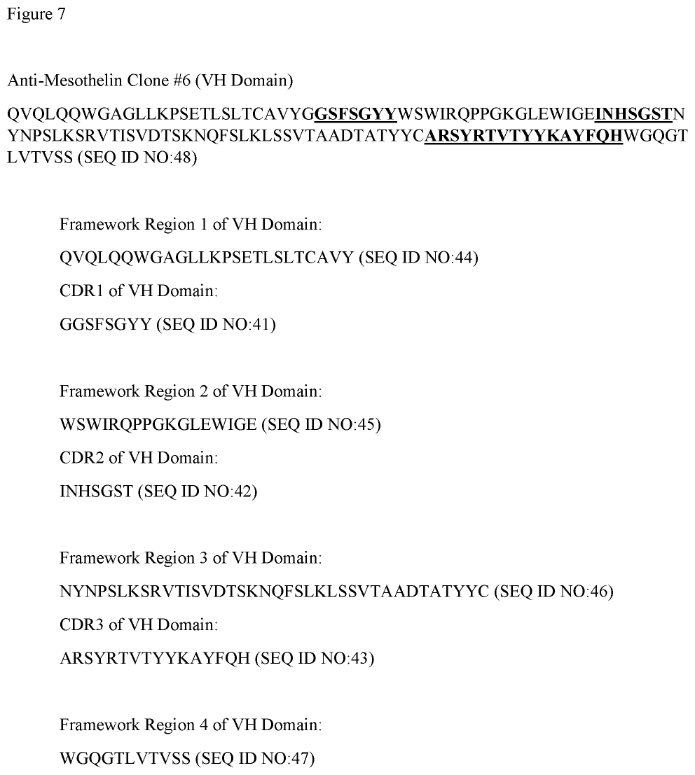

In one embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:41 (or a variant of SEQ ID NO:41 with one or two amino acid modifications), a CDR2 having the amino acid sequence set forth in SEQ ID NO:42 (or a variant of SEQ ID NO:42 with one or two amino acid modifications), and a CDR3 having the amino acid sequence set forth in SEQ ID NO:43 (or a variant of SEQ ID NO:43 with one or two amino acid modifications). An example of such an antibody domain having these CDRs and the ability to bind to a mesothelin polypeptide (e.g., a human mesothelin polypeptide) includes, without limitation, the VH domain set forth in FIG. 7.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:41 (or a variant of SEQ ID NO:41 with one or two amino acid modifications), a CDR2 having the amino acid sequence set forth in SEQ ID NO:42 (or a variant of SEQ ID NO:42 with one or two amino acid modifications), and a CDR3 having the amino acid sequence set forth in SEQ ID NO:43 (or a variant of SEQ ID NO:43 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:44 (or a variant of SEQ ID NO:44 with one, two, three, four, five, six, seven, eight, nine, ten, or more amino acid modifications), a framework region 2 having the amino acid sequence set forth in SEQ ID NO:45 (or a variant of SEQ ID NO:45 with one, two, three, four, five, six, seven, eight, nine, ten, or more amino acid modifications), a framework region 3 having the amino acid sequence set forth in SEQ ID NO:46 (or a variant of SEQ ID NO:46 with one, two, three, four, five, six, seven, eight, nine, ten, or more amino acid modifications), and a framework region 4 having the amino acid sequence set forth in SEQ ID NO:47 (or a variant of SEQ ID NO:47 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 7 can be designed to include framework regions as set forth in FIG. 7 or can be designed to include one or more framework regions from another antibody or antibody fragment. For example, an antibody domain (e.g., a VH domain) can be designed to include the three CDRs set forth in FIG. 7 and the framework regions set forth in FIG. 7 except that framework region 1 having the amino acid set forth in SEQ ID NO:44 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 7, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:48. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:48. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:48.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:48, provided that the heavy chain variable domain includes the amino acid sequences set forth in SEQ ID NOs:41, 42, and 43. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:48, provided that the heavy chain variable domain includes the amino acid sequences set forth in SEQ ID NOs:41, 42, and 43.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:48 or the amino acid set forth in SEQ ID NO:48 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:48 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:41, 42, and 43.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:41, (ii) a CDR2 that comprises, consists essentially of, or consists of the amino acid sequence set forth in SEQ ID NO:42, and (iii) a CDR3 that comprises, consists essentially of, or consists of the amino acid sequence set forth in SEQ ID NO:43. As used herein, a “CDR1 that consists essentially of the amino acid sequence set forth in SEQ ID NO:41” is a CDR1 that has zero, one, or two amino acid substitutions within SEQ ID NO:41, that has zero, one, two, three, four, or five amino acid residues directly preceding SEQ ID NO:41, and/or that has zero, one, two, three, four, or five amino acid residues directly following SEQ ID NO:41, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:41 include, without limitation, those set forth in Table 16.

TABLE 16
Exemplary CDR1s that consist essentially of
the amino acid sequence set forth
in SEQ ID NO: 41.

| Sequence | SEQ ID NO: |
|---|---|
| GYTFTGYY | 321 |
| GYTFTSYA | 322 |
| GYTFTSYG | 323 |
| GFTFTSSA | 324 |
| GFTFSSYW | 325 |
| GFTFDDYA | 326 |
| GFTFSSYA | 327 |
| GFTFSSYG | 328 |
| GFTFSSSA | 329 |
| GFTFSDHY | 330 |

As used herein, a “CDR2 that consists essentially of the amino acid sequence set forth in SEQ ID NO:42” is a CDR2 that has zero or one amino acid substitutions within SEQ ID NO:42, that has zero, one, two, three, four, or five amino acid residues directly preceding SEQ ID NO:42, and/or that has zero, one, two, three, four, or five amino acid residues directly following SEQ ID NO:42, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:42 include, without limitation, those set forth in Table 17.

TABLE 17
Exemplary CDR2s that consist essentially
of the amino acid sequence set forth
in SEQ ID NO: 42.

| Sequence | SEQ ID NO: |
|---|---|
| INPNSGGT | 331 |
| ISAYNGNT | 332 |
| ISSSGST | 333 |
| IGTAGDT | 334 |
| ISSSSSYI | 335 |
| ISGSGGST | 336 |
| ISYDGSNK | 337 |
| IYSGGST | 338 |
| IYHSGST | 339 |
| ISGSGGST | 340 |

As used herein, a “CDR3 that consists essentially of the amino acid sequence set forth in SEQ ID NO:43” is a CDR3 that has zero, one, or two amino acid substitutions within SEQ ID NO:43, that has zero, one, two, three, four, or five amino acid residues directly preceding SEQ ID NO:43, and/or that has zero, one, two, three, four, or five amino acid residues directly following SEQ ID NO:43, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:43 include, without limitation, those set forth in Table 18.

TABLE 18
Exemplary CDR3s that consist essentially of
the amino acid sequence set forth
in SEQ ID NO: 43.

| Sequence | SEQ ID NO: |
|---|---|
| ARRSYRTVTYYKAYFQH | 341 |
| ATSYRTVTYYKAYFQH | 342 |
| AASYRTVTYYKAYFQH | 343 |
| VRSYRTVTYYKAYFQH | 344 |
| AHRSYRTVTYYKAYFQH | 345 |
| ARISYRTVTYYKAYFQH | 346 |
| AKDSYRTVTYYKAYFQH | 347 |
| TTSYRTVTYYKAYFQH | 348 |
| SRSYRTVTYYKAYFQH | 349 |
| AKSYRTVTYYKAYFQH | 350 |

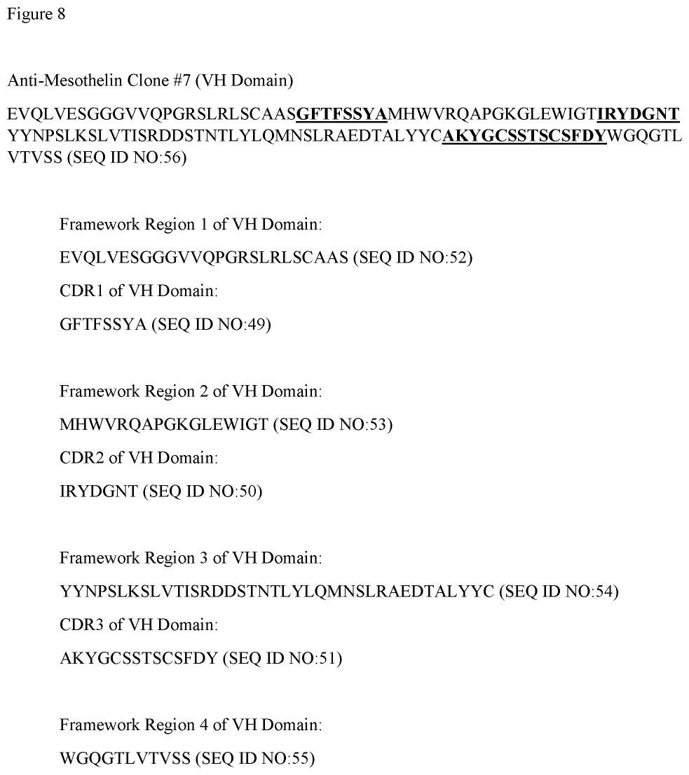

In another embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:49 (or a variant of SEQ ID NO:49 with one or two amino acid modifications), a CDR2 having the amino acid sequence set forth in SEQ ID NO:50 (or a variant of SEQ ID NO:50 with one or two amino acid modifications), and a CDR3 having the amino acid sequence set forth in SEQ ID NO:51 (or a variant of SEQ ID NO:51 with one or two amino acid modifications). An example of such an antibody domain having these CDRs and the ability to bind to a mesothelin polypeptide (e.g., a human mesothelin polypeptide) includes, without limitation, the VH domain set forth in FIG. 8.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:49 (or a variant of SEQ ID NO:49 with one or two amino acid modifications), a CDR2 having the amino acid sequence set forth in SEQ ID NO:50 (or a variant of SEQ ID NO:50 with one or two amino acid modifications), and a CDR3 having the amino acid sequence set forth in SEQ ID NO:51 (or a variant of SEQ ID NO:51 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:52 (or a variant of SEQ ID NO:52 with one, two, three, four, five, six, seven, eight, nine, ten, or more amino acid modifications), a framework region 2 having the amino acid sequence set forth in SEQ ID NO:53 (or a variant of SEQ ID NO:53 with one, two, three, four, five, six, seven, eight, nine, ten, or more amino acid modifications), a framework region 3 having the amino acid sequence set forth in SEQ ID NO:54 (or a variant of SEQ ID NO:54 with one, two, three, four, five, six, seven, eight, nine, ten, or more amino acid modifications), and a framework region 4 having the amino acid sequence set forth in SEQ ID NO:55 (or a variant of SEQ ID NO:55 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 8 can be designed to include framework regions as set forth in FIG. 8 or can be designed to include one or more framework regions from another antibody or antibody fragment. For example, an antibody domain (e.g., a VH domain) can be designed to include the three CDRs set forth in FIG. 8 and the framework regions set forth in FIG. 8 except that framework region 1 having the amino acid set forth in SEQ ID NO:52 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:60, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 8, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:56. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:56. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:56.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:56, provided that the heavy chain variable domain includes the amino acid sequences set forth in SEQ ID NOs:49, 50, and 51. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:56, provided that the heavy chain variable domain includes the amino acid sequences set forth in SEQ ID NOs:49, 50, and 51.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:56 or the amino acid set forth in SEQ ID NO:56 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:56 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:49, 50, and 51.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:49, (ii) a CDR2 that comprises, consists essentially of, or consists of the amino acid sequence set forth in SEQ ID NO:50, and (iii) a CDR3 that comprises, consists essentially of, or consists of the amino acid sequence set forth in SEQ ID NO:51. As used herein, a “CDR1 that consists essentially of the amino acid sequence set forth in SEQ ID NO:49” is a CDR1 that has zero, one, or two amino acid substitutions within SEQ ID NO:49, that has zero, one, two, three, four, or five amino acid residues directly preceding SEQ ID NO:49, and/or that has zero, one, two, three, four, or five amino acid residues directly following SEQ ID NO:49, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:49 include, without limitation, those set forth in Table 19.

TABLE 19
Exemplary CDR1s that consist essentially of
the amino acid sequence set forth
in SEQ ID NO: 49.

| Sequence | SEQ ID NO: |
|---|---|
| GYTFTGYY | 351 |
| GYTFTSYA | 352 |
| GYTFTSYG | 353 |
| GFTFTSSA | 354 |
| GFTFSSYW | 355 |
| GFTFDDYA | 356 |
| GFTFSSYD | 357 |
| GFTFSSYG | 358 |
| GFTFSSSA | 359 |
| GFTFSDHY | 360 |

As used herein, a “CDR2 that consists essentially of the amino acid sequence set forth in SEQ ID NO:50” is a CDR2 that has zero, one, or two amino acid substitutions within SEQ ID NO:50, that has zero, one, two, three, four, or five amino acid residues directly preceding SEQ ID NO:50, and/or that has zero, one, two, three, four, or five amino acid residues directly following SEQ ID NO:50, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:50 include, without limitation, those set forth in Table 20.

TABLE 20
Exemplary CDR2s that consist essentially of
the amino acid sequence set forth
in SEQ ID NO: 50.

| Sequence | SEQ ID NO: |
|---|---|
| INPNSGGT | 361 |
| ISAYNGNT | 362 |
| ISSSGST | 363 |
| IGTAGDT | 364 |
| ISSSSSYI | 365 |
| ISGSGGST | 366 |
| ISYDGSNK | 367 |
| IYSGGST | 368 |
| IYHSGST | 369 |
| ISGSGGST | 370 |

As used herein, a “CDR3 that consists essentially of the amino acid sequence set forth in SEQ ID NO:51” is a CDR3 that has zero, one, or two amino acid substitutions within SEQ ID NO:51, that has zero, one, two, three, four, or five amino acid residues directly preceding SEQ ID NO:51, and/or that has zero, one, two, three, four, or five amino acid residues directly following SEQ ID NO:51, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:51 include, without limitation, those set forth in Table 21.

TABLE 21
Exemplary CDR3s that consist essentially of
the amino acid sequence set forth
in SEQ ID NO: 51.

| Sequence | SEQ ID NO: |
|---|---|
| ARRYGCSSTSCSFDY | 371 |
| ATYGCSSTSCSFDY | 372 |
| AAYGCSSTSCSFDY | 373 |
| VRYGCSSTSCSFDY | 374 |
| AHRYGCSSTSCSFDY | 375 |
| ARIYGCSSTSCSFDY | 376 |
| AKDYGCSSTSCSFDY | 377 |
| TTYGCSSTSCSFDY | 378 |
| SRYGCSSTSCSFDY | 379 |
| ARKYGCSSTSCSFDY | 380 |

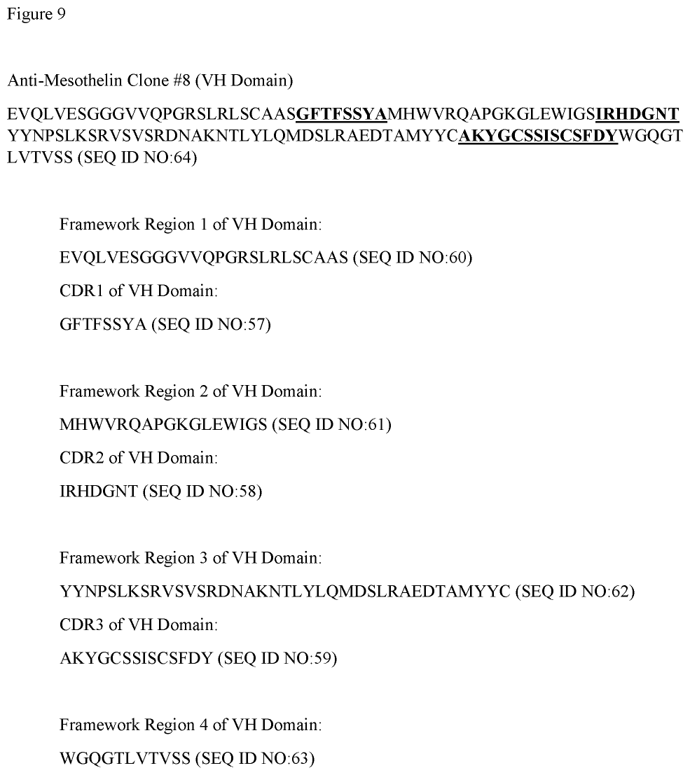

In another embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:57 (or a variant of SEQ ID NO:57 with one or two amino acid modifications), a CDR2 having the amino acid sequence set forth in SEQ ID NO:58 (or a variant of SEQ ID NO:58 with one or two amino acid modifications), and a CDR3 having the amino acid sequence set forth in SEQ ID NO:59 (or a variant of SEQ ID NO:59 with one or two amino acid modifications). An example of such an antibody domain having these CDRs and the ability to bind to a mesothelin polypeptide (e.g., a human mesothelin polypeptide) includes, without limitation, the VH domain set forth in FIG. 9.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:57 (or a variant of SEQ ID NO:57 with one or two amino acid modifications), a CDR2 having the amino acid sequence set forth in SEQ ID NO:58 (or a variant of SEQ ID NO:58 with one or two amino acid modifications), and a CDR3 having the amino acid sequence set forth in SEQ ID NO:59 (or a variant of SEQ ID NO:59 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:60 (or a variant of SEQ ID NO:60 with one, two, three, four, five, six, seven, eight, nine, ten, or more amino acid modifications), a framework region 2 having the amino acid sequence set forth in SEQ ID NO:61 (or a variant of SEQ ID NO:61 with one, two, three, four, five, six, seven, eight, nine, ten, or more amino acid modifications), a framework region 3 having the amino acid sequence set forth in SEQ ID NO:62 (or a variant of SEQ ID NO:62 with one, two, three, four, five, six, seven, eight, nine, ten, or more amino acid modifications), and a framework region 4 having the amino acid sequence set forth in SEQ ID NO:63 (or a variant of SEQ ID NO:63 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 9 can be designed to include framework regions as set forth in FIG. 9 or can be designed to include one or more framework regions from another antibody or antibody fragment. For example, an antibody domain (e.g., a VH domain) can be designed to include the three CDRs set forth in FIG. 9 and the framework regions set forth in FIG. 9 except that framework region 1 having the amino acid set forth in SEQ ID NO:60 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:68, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 9, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:64. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:64. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:64.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:64, provided that the heavy chain variable domain includes the amino acid sequences set forth in SEQ ID NOs:57, 58, and 59. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:64, provided that the heavy chain variable domain includes the amino acid sequences set forth in SEQ ID NOs:57, 58, and 59.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:64 or the amino acid set forth in SEQ ID NO:64 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:64 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:57, 58, and 59.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:57, (ii) a CDR2 that comprises, consists essentially of, or consists of the amino acid sequence set forth in SEQ ID NO:58, and (iii) a CDR3 that comprises, consists essentially of, or consists of the amino acid sequence set forth in SEQ ID NO:59. As used herein, a “CDR1 that consists essentially of the amino acid sequence set forth in SEQ ID NO:57” is a CDR1 that has zero, one, or two amino acid substitutions within SEQ ID NO:57, that has zero, one, two, three, four, or five amino acid residues directly preceding SEQ ID NO:57, and/or that has zero, one, two, three, four, or five amino acid residues directly following SEQ ID NO:57, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:57 include, without limitation, those set forth in Table 22.

TABLE 22
Exemplary CDR1s that consist essentially
of the amino acid sequence set forth in
SEQ ID NO: 57.

| Sequence | SEQ ID NO: |
|---|---|
| GYTFTGYY | 381 |
| GYTFTSYA | 382 |
| GYTFTSYG | 383 |
| GFTFTSSA | 384 |
| GFTFSSYW | 385 |
| GFTFDDYA | 386 |
| GFTFSSYD | 387 |
| GFTFSSYG | 388 |
| GFTFSSSA | 389 |
| GFTFSDHY | 390 |

As used herein, a “CDR2 that consists essentially of the amino acid sequence set forth in SEQ ID NO:58” is a CDR2 that has zero, one, or two amino acid substitutions within SEQ ID NO:58, that has zero, one, two, three, four, or five amino acid residues directly preceding SEQ ID NO:58, and/or that has zero, one, two, three, four, or five amino acid residues directly following SEQ ID NO:58, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:58 include, without limitation, those set forth in Table 23.

TABLE 23
Exemplary CDR2s that consist essentially
of the amino acid sequence set forth in
SEQ ID NO: 58.

| Sequence | SEQ ID NO: |
|---|---|
| INPNSGGT | 391 |
| ISAYNGNT | 392 |
| ISSSGST | 393 |
| IGTAGDT | 394 |
| ISSSSSYI | 395 |
| ISGSGGST | 396 |
| ISYDGSNK | 397 |
| IYSGGST | 398 |
| IYHSGST | 399 |
| ISGSGGST | 400 |

As used herein, a “CDR3 that consists essentially of the amino acid sequence set forth in SEQ ID NO:59” is a CDR3 that has zero, one, or two amino acid substitutions within SEQ ID NO:59, that has zero, one, two, three, four, or five amino acid residues directly preceding SEQ ID NO:59, and/or that has zero, one, two, three, four, or five amino acid residues directly following SEQ ID NO:59, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:59 include, without limitation, those set forth in Table 24.

TABLE 24
Exemplary CDR3s that consist essentially
of the amino acid sequence set forth
in SEQ ID NO: 59.

| Sequence | SEQ ID NO: |
|---|---|
| ARRYGCSSISCSFDY | 401 |
| ATYGCSSISCSFDY | 402 |
| AAYGCSSISCSFDY | 403 |
| VRYGCSSISCSFDY | 404 |
| AHRYGCSSISCSFDY | 405 |
| ARIYGCSSISCSFDY | 406 |
| AKDYGCSSISCSFDY | 407 |
| TTYGCSSISCSFDY | 408 |
| SRYGCSSISCSFDY | 409 |
| ARKYGCSSISCSFDY | 410 |

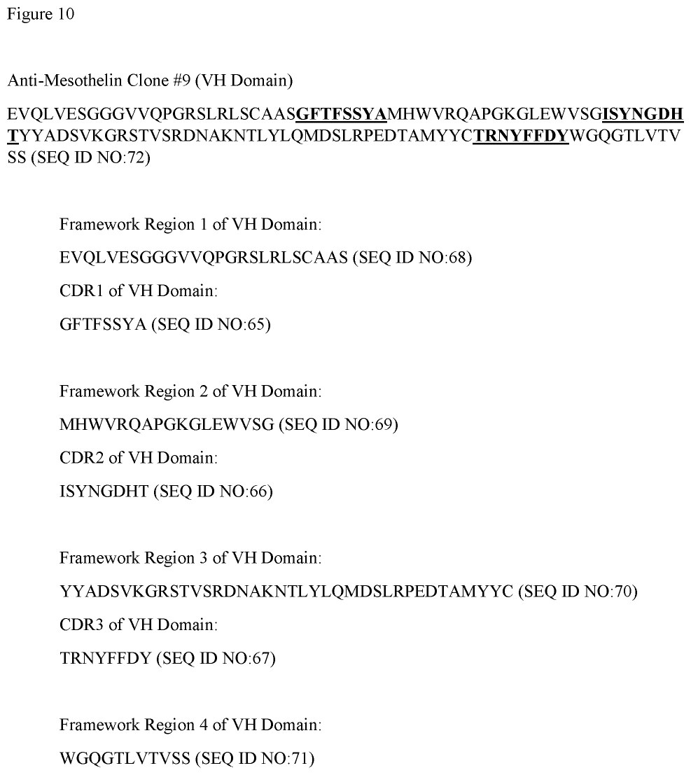

In another embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:65 (or a variant of SEQ ID NO:65 with one or two amino acid modifications), a CDR2 having the amino acid sequence set forth in SEQ ID NO:66 (or a variant of SEQ ID NO:66 with one or two amino acid modifications), and a CDR3 having the amino acid sequence set forth in SEQ ID NO:67 (or a variant of SEQ ID NO:67 with one or two amino acid modifications). An example of such an antibody domain having these CDRs and the ability to bind to a mesothelin polypeptide (e.g., a human mesothelin polypeptide) includes, without limitation, the VH domain set forth in FIG. 10.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:65 (or a variant of SEQ ID NO:65 with one or two amino acid modifications), a CDR2 having the amino acid sequence set forth in SEQ ID NO:66 (or a variant of SEQ ID NO:66 with one or two amino acid modifications), and a CDR3 having the amino acid sequence set forth in SEQ ID NO:67 (or a variant of SEQ ID NO:67 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:68 (or a variant of SEQ ID NO:68 with one, two, three, four, five, six, seven, eight, nine, ten, or more amino acid modifications), a framework region 2 having the amino acid sequence set forth in SEQ ID NO:69 (or a variant of SEQ ID NO:69 with one, two, three, four, five, six, seven, eight, nine, ten, or more amino acid modifications), a framework region 3 having the amino acid sequence set forth in SEQ ID NO:70 (or a variant of SEQ ID NO:70 with one, two, three, four, five, six, seven, eight, nine, ten, or more amino acid modifications), and a framework region 4 having the amino acid sequence set forth in SEQ ID NO:71 (or a variant of SEQ ID NO:71 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 10 can be designed to include framework regions as set forth in FIG. 10 or can be designed to include one or more framework regions from another antibody or antibody fragment. For example, an antibody domain (e.g., a VH domain) can be designed to include the three CDRs set forth in FIG. 10 and the framework regions set forth in FIG. 10 except that framework region 1 having the amino acid set forth in SEQ ID NO:68 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:76, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 10, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:72. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:72. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:72.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:72, provided that the heavy chain variable domain includes the amino acid sequences set forth in SEQ ID NOs:65, 66, and 67. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:72, provided that the heavy chain variable domain includes the amino acid sequences set forth in SEQ ID NOs:65, 66, and 67.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:72 or the amino acid set forth in SEQ ID NO:72 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:72 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:65, 66, and 67.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:65, (ii) a CDR2 that comprises, consists essentially of, or consists of the amino acid sequence set forth in SEQ ID NO:66, and (iii) a CDR3 that comprises, consists essentially of, or consists of the amino acid sequence set forth in SEQ ID NO:67. As used herein, a “CDR1 that consists essentially of the amino acid sequence set forth in SEQ ID NO:65” is a CDR1 that has zero, one, or two amino acid substitutions within SEQ ID NO:65, that has zero, one, two, three, four, or five amino acid residues directly preceding SEQ ID NO:65, and/or that has zero, one, two, three, four, or five amino acid residues directly following SEQ ID NO:65, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:65 include, without limitation, those set forth in Table 25.

TABLE 25
Exemplary CDR1s that consist essentially
of the amino acid sequence set forth
in SEQ ID NO: 65.

| Sequence | SEQ ID NO: |
|---|---|
| GYTFTGYY | 411 |
| GYTFTSYA | 412 |
| GYTFTSYG | 413 |
| GFTFTSSA | 414 |
| GFTFSSYW | 415 |
| GFTFDDYA | 416 |
| GFTFSSYD | 417 |
| GFTFSSYG | 418 |
| GFTFSSSA | 419 |
| GFTFSDHY | 420 |

As used herein, a “CDR2 that consists essentially of the amino acid sequence set forth in SEQ ID NO:66” is a CDR2 that has zero, one, or two amino acid substitutions within SEQ ID NO:66, that has zero, one, two, three, four, or five amino acid residues directly preceding SEQ ID NO:66, and/or that has zero, one, two, three, four, or five amino acid residues directly following SEQ ID NO:66, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:66 include, without limitation, those set forth in Table 26.

TABLE 26
Exemplary CDR2s that consist essentially
of the amino acid sequence set forth
in SEQ ID NO: 66.

| Sequence | SEQ ID NO: |
|---|---|
| INPNSGGT | 421 |
| ISAYNGNT | 422 |
| ISSSGST | 423 |
| IGTAGDT | 424 |
| ISSSSSYI | 425 |
| ISGSGGST | 426 |
| ISYDGSNK | 427 |
| IYSGGST | 428 |
| IYHSGST | 429 |
| ISGSGGST | 430 |

As used herein, a “CDR3 that consists essentially of the amino acid sequence set forth in SEQ ID NO:67” is a CDR3 that has zero, one, or two amino acid substitutions within SEQ ID NO:67, that has zero, one, two, three, four, or five amino acid residues directly preceding SEQ ID NO:67, and/or that has zero, one, two, three, four, or five amino acid residues directly following SEQ ID NO:67, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:67 include, without limitation, those set forth in Table 27.

TABLE 27
Exemplary CDR3s that consist essentially
of the amino acid sequence set forth
in SEQ ID NO: 67.

| Sequence | SEQ ID NO: |
|---|---|
| ARRTRNYFFDY | 431 |
| ATTRNYFFDY | 432 |
| AATRNYFFDY | 433 |
| VRTRNYFFDY | 434 |
| AHRTRNYFFDY | 435 |
| ARITRNYFFDY | 436 |
| AKDTRNYFFDY | 437 |
| TTTRNYFFDY | 438 |
| SRTRNYFFDY | 439 |
| AKTRNYFFDY | 440 |

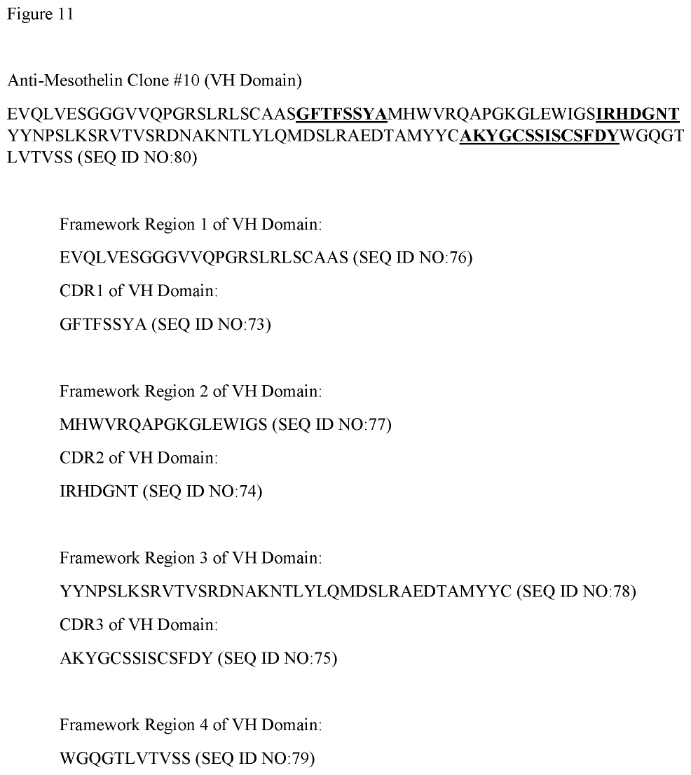

In another embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:73 (or a variant of SEQ ID NO:73 with one or two amino acid modifications), a CDR2 having the amino acid sequence set forth in SEQ ID NO:74 (or a variant of SEQ ID NO:74 with one or two amino acid modifications), and a CDR3 having the amino acid sequence set forth in SEQ ID NO:75 (or a variant of SEQ ID NO:75 with one or two amino acid modifications). An example of such an antibody domain having these CDRs and the ability to bind to a mesothelin polypeptide (e.g., a human mesothelin polypeptide) includes, without limitation, the VH domain set forth in FIG. 11.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:73 (or a variant of SEQ ID NO:73 with one or two amino acid modifications), a CDR2 having the amino acid sequence set forth in SEQ ID NO:74 (or a variant of SEQ ID NO:74 with one or two amino acid modifications), and a CDR3 having the amino acid sequence set forth in SEQ ID NO:75 (or a variant of SEQ ID NO:75 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:76 (or a variant of SEQ ID NO:76 with one, two, three, four, five, six, seven, eight, nine, ten, or more amino acid modifications), a framework region 2 having the amino acid sequence set forth in SEQ ID NO:77 (or a variant of SEQ ID NO:77 with one, two, three, four, five, six, seven, eight, nine, ten, or more amino acid modifications), a framework region 3 having the amino acid sequence set forth in SEQ ID NO:78 (or a variant of SEQ ID NO:78 with one, two, three, four, five, six, seven, eight, nine, ten, or more amino acid modifications), and a framework region 4 having the amino acid sequence set forth in SEQ ID NO:79 (or a variant of SEQ ID NO:79 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 11 can be designed to include framework regions as set forth in FIG. 11 or can be designed to include one or more framework regions from another antibody or antibody fragment. For example, an antibody domain (e.g., a VH domain) can be designed to include the three CDRs set forth in FIG. 11 and the framework regions set forth in FIG. 11 except that framework region 1 having the amino acid set forth in SEQ ID NO:76 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:84, or a framework region 1 having the amino acid set forth in SEQ ID NO:92. In another example, an Fab or scFv can be designed to include (a) the three CDRs set forth in FIG. 11, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:80. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:80. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:80.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:80, provided that the heavy chain variable domain includes the amino acid sequences set forth in SEQ ID NOs:73, 74, and 75. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:80, provided that the heavy chain variable domain includes the amino acid sequences set forth in SEQ ID NOs:73, 74, and 75.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:80 or the amino acid set forth in SEQ ID NO:80 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:80 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:73, 74, and 75.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:73, (ii) a CDR2 that comprises, consists essentially of, or consists of the amino acid sequence set forth in SEQ ID NO:74, and (iii) a CDR3 that comprises, consists essentially of, or consists of the amino acid sequence set forth in SEQ ID NO:75. As used herein, a “CDR1 that consists essentially of the amino acid sequence set forth in SEQ ID NO:73” is a CDR1 that has zero, one, or two amino acid substitutions within SEQ ID NO:73, that has zero, one, two, three, four, or five amino acid residues directly preceding SEQ ID NO:73, and/or that has zero, one, two, three, four, or five amino acid residues directly following SEQ ID NO:73, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:73 include, without limitation, those set forth in Table 28.

TABLE 28
Exemplary CDR1s that consist essentially
of the amino acid sequence set forth
in SEQ ID NO: 73.

| Sequence | SEQ ID NO: |
|---|---|
| GYTFTGYY | 441 |
| GYTFTSYA | 442 |
| GYTFTSYG | 443 |
| GFTFTSSA | 444 |
| GFTFSSYW | 445 |
| GFTFDDYA | 446 |
| GFTFSSYD | 447 |
| GFTFSSYG | 448 |
| GFTFSSSA | 449 |
| GFTFSDHY | 450 |

As used herein, a “CDR2 that consists essentially of the amino acid sequence set forth in SEQ ID NO:74” is a CDR2 that has zero, one, or two amino acid substitutions within SEQ ID NO:74, that has zero, one, two, three, four, or five amino acid residues directly preceding SEQ ID NO:74, and/or that has zero, one, two, three, four, or five amino acid residues directly following SEQ ID NO:74, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:74 include, without limitation, those set forth in Table 29.

TABLE 29
Exemplary CDR2s that consist essentially
of the amino acid sequence set forth
in SEQ ID NO: 74.

| Sequence | SEQ ID NO: |
|---|---|
| INPNSGGT | 451 |
| ISAYNGNT | 452 |
| ISSSGST | 453 |
| IGTAGDT | 454 |
| ISSSSSYI | 455 |
| ISGSGGST | 456 |
| ISYDGSNK | 457 |
| IYSGGST | 458 |
| IYHSGST | 459 |
| ISGSGGST | 460 |

As used herein, a “CDR3 that consists essentially of the amino acid sequence set forth in SEQ ID NO:75” is a CDR3 that has zero, one, or two amino acid substitutions within SEQ ID NO:75, that has zero, one, two, three, four, or five amino acid residues directly preceding SEQ ID NO:75, and/or that has zero, one, two, three, four, or five amino acid residues directly following SEQ ID NO:75, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:75 include, without limitation, those set forth in Table 30.

TABLE 30
Exemplary CDR3s that consist essentially
of the amino acid sequence set forth
in SEQ ID NO: 75.

| Sequence | SEQ ID NO: |
|---|---|
| ARRYGCSSISCSFDY | 461 |
| ATYGCSSISCSFDY | 462 |
| AAYGCSSISCSFDY | 463 |
| VRYGCSSISCSFDY | 464 |
| AHRYGCSSISCSFDY | 465 |
| ARIYGCSSISCSFDY | 466 |
| AKDYGCSSISCSFDY | 467 |
| TTYGCSSISCSFDY | 468 |
| SRYGCSSISCSFDY | 469 |
| ARKYGCSSISCSFDY | 470 |

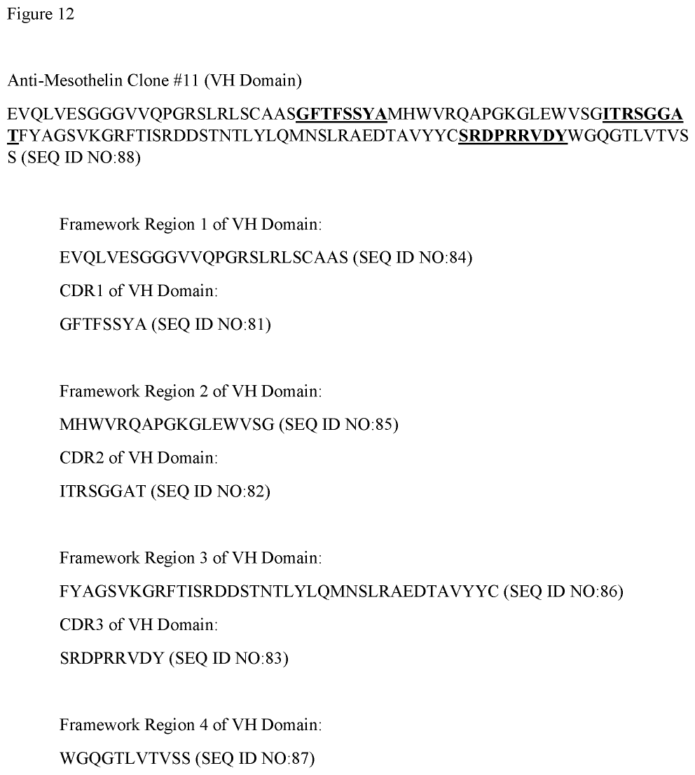

In another embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:81 (or a variant of SEQ ID NO:81 with one or two amino acid modifications), a CDR2 having the amino acid sequence set forth in SEQ ID NO:82 (or a variant of SEQ ID NO:82 with one or two amino acid modifications), and a CDR3 having the amino acid sequence set forth in SEQ ID NO:83 (or a variant of SEQ ID NO:83 with one or two amino acid modifications). An example of such an antibody domain having these CDRs and the ability to bind to a mesothelin polypeptide (e.g., a human mesothelin polypeptide) includes, without limitation, the VH domain set forth in FIG. 12.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:81 (or a variant of SEQ ID NO:81 with one or two amino acid modifications), a CDR2 having the amino acid sequence set forth in SEQ ID NO:82 (or a variant of SEQ ID NO:82 with one or two amino acid modifications), and a CDR3 having the amino acid sequence set forth in SEQ ID NO:83 (or a variant of SEQ ID NO:83 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:84 (or a variant of SEQ ID NO:84 with one, two, three, four, five, six, seven, eight, nine, ten, or more amino acid modifications), a framework region 2 having the amino acid sequence set forth in SEQ ID NO:85 (or a variant of SEQ ID NO:85 with one, two, three, four, five, six, seven, eight, nine, ten, or more amino acid modifications), a framework region 3 having the amino acid sequence set forth in SEQ ID NO:86 (or a variant of SEQ ID NO:86 with one, two, three, four, five, six, seven, eight, nine, ten, or more amino acid modifications), and a framework region 4 having the amino acid sequence set forth in SEQ ID NO:87 (or a variant of SEQ ID NO:87 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 12 can be designed to include framework regions as set forth in FIG. 12 or can be designed to include one or more framework regions from another antibody or antibody fragment. For example, an antibody domain (e.g., a VH domain) can be designed to include the three CDRs set forth in FIG. 12 and the framework regions set forth in FIG. 12 except that framework region 1 having the amino acid set forth in SEQ ID NO:84 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, or a framework region 1 having the amino acid set forth in SEQ ID NO:92. In another example, an Fab or scFv can be designed to include (a) the three CDRs set forth in FIG. 12, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:88. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:88. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:88.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:88, provided that the heavy chain variable domain includes the amino acid sequences set forth in SEQ ID NOs:81, 82, and 83. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:88, provided that the heavy chain variable domain includes the amino acid sequences set forth in SEQ ID NOs:81, 82, and 83.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:88 or the amino acid set forth in SEQ ID NO:88 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:88 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:81, 82, and 83.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:81, (ii) a CDR2 that comprises, consists essentially of, or consists of the amino acid sequence set forth in SEQ ID NO:82, and (iii) a CDR3 that comprises, consists essentially of, or consists of the amino acid sequence set forth in SEQ ID NO:83. As used herein, a “CDR1 that consists essentially of the amino acid sequence set forth in SEQ ID NO:81” is a CDR1 that has zero, one, or two amino acid substitutions within SEQ ID NO:81, that has zero, one, two, three, four, or five amino acid residues directly preceding SEQ ID NO:81, and/or that has zero, one, two, three, four, or five amino acid residues directly following SEQ ID NO:81, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:81 include, without limitation, those set forth in Table 31.

TABLE 31
Exemplary CDR1s that consist essentially
of the amino acid sequence set forth
in SEQ ID NO: 81.

| Sequence | SEQ ID NO: |
|---|---|
| GYTFTGYY | 471 |
| GYTFTSYA | 472 |
| GYTFTSYG | 473 |
| GFTFTSSA | 474 |
| GFTFSSYW | 475 |
| GFTFDDYA | 476 |
| GFTFSSYD | 477 |
| GFTFSSYG | 478 |
| GFTFSSSA | 479 |
| GFTFSDHY | 480 |

As used herein, a “CDR2 that consists essentially of the amino acid sequence set forth in SEQ ID NO:82” is a CDR2 that has zero, one, or two amino acid substitutions within SEQ ID NO:82, that has zero, one, two, three, four, or five amino acid residues directly preceding SEQ ID NO:82, and/or that has zero, one, two, three, four, or five amino acid residues directly following SEQ ID NO:82, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:82 include, without limitation, those set forth in Table 32.

TABLE 32
Exemplary CDR2s that consist essentially
of the amino acid sequence set forth
in SEQ ID NO: 82.

| Sequence | SEQ ID NO: |
|---|---|
| INPNSGGT | 481 |
| ISAYNGNT | 482 |
| ISSSGST | 483 |
| IGTAGDT | 484 |
| ISSSSSYI | 485 |
| ISGSGGST | 486 |
| ISYDGSNK | 487 |
| IYSGGST | 488 |
| IYHSGST | 489 |
| ISGSGGST | 490 |

As used herein, a “CDR3 that consists essentially of the amino acid sequence set forth in SEQ ID NO:83” is a CDR3 that has zero, one, or two amino acid substitutions within SEQ ID NO:83, that has zero, one, two, three, four, or five amino acid residues directly preceding SEQ ID NO:83, and/or that has zero, one, two, three, four, or five amino acid residues directly following SEQ ID NO:83, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:83 include, without limitation, those set forth in Table 33.

TABLE 33
Exemplary CDR3s that consist essentially
of the amino acid sequence set forth
in SEQ ID NO: 83.

| Sequence | SEQ ID NO: |
|---|---|
| ARRSRDPRRVDY | 491 |
| ATSRDPRRVDY | 492 |
| AASRDPRRVDY | 493 |
| VRSRDPRRVDY | 494 |
| AHRSRDPRRVDY | 495 |
| ARISRDPRRVDY | 496 |
| AKDSRDPRRVDY | 497 |
| TTSRDPRRVDY | 498 |
| SRSRDPRRVDY | 499 |
| AKSRDPRRVDY | 500 |

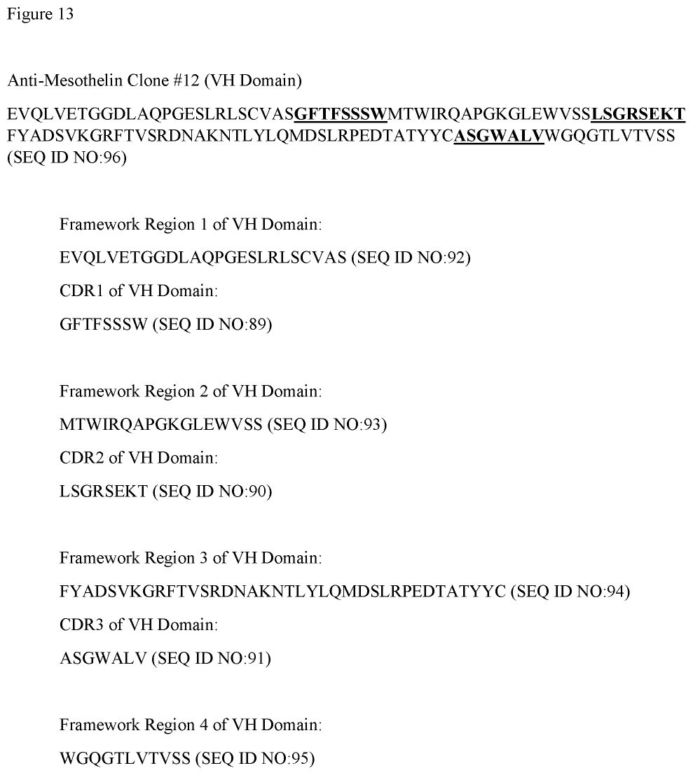

In another embodiment, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having a CDR1 having the amino acid sequence set forth in SEQ ID NO:89 (or a variant of SEQ ID NO:89 with one or two amino acid modifications), a CDR2 having the amino acid sequence set forth in SEQ ID NO:90 (or a variant of SEQ ID NO:90 with one or two amino acid modifications), and a CDR3 having the amino acid sequence set forth in SEQ ID NO:91 (or a variant of SEQ ID NO:91 with one or two amino acid modifications). An example of such an antibody domain having these CDRs and the ability to bind to a mesothelin polypeptide (e.g., a human mesothelin polypeptide) includes, without limitation, the VH domain set forth in FIG. 13.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) and having a heavy chain variable domain having a CDR1 having the amino acid sequence set forth in SEQ ID NO:89 (or a variant of SEQ ID NO:89 with one or two amino acid modifications), a CDR2 having the amino acid sequence set forth in SEQ ID NO:90 (or a variant of SEQ ID NO:90 with one or two amino acid modifications), and a CDR3 having the amino acid sequence set forth in SEQ ID NO:91 (or a variant of SEQ ID NO:91 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:92 (or a variant of SEQ ID NO:92 with one, two, three, four, five, six, seven, eight, nine, ten, or more amino acid modifications), a framework region 2 having the amino acid sequence set forth in SEQ ID NO:93 (or a variant of SEQ ID NO:93 with one, two, three, four, five, six, seven, eight, nine, ten, or more amino acid modifications), a framework region 3 having the amino acid sequence set forth in SEQ ID NO:94 (or a variant of SEQ ID NO:94 with one, two, three, four, five, six, seven, eight, nine, ten, or more amino acid modifications), and a framework region 4 having the amino acid sequence set forth in SEQ ID NO:95 (or a variant of SEQ ID NO:95 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 13 can be designed to include framework regions as set forth in FIG. 13 or can be designed to include one or more framework regions from another antibody or antibody fragment. For example, an antibody domain (e.g., a VH domain) can be designed to include the three CDRs set forth in FIG. 13 and the framework regions set forth in FIG. 13 except that framework region 1 having the amino acid set forth in SEQ ID NO:92 is replaced with a framework region 1 having the amino acid set forth in SEQ ID NO:4, a framework region 1 having the amino acid set forth in SEQ ID NO:12, a framework region 1 having the amino acid set forth in SEQ ID NO:20, a framework region 1 having the amino acid set forth in SEQ ID NO:28, a framework region 1 having the amino acid set forth in SEQ ID NO:36, a framework region 1 having the amino acid set forth in SEQ ID NO:44, a framework region 1 having the amino acid set forth in SEQ ID NO:52, a framework region 1 having the amino acid set forth in SEQ ID NO:60, a framework region 1 having the amino acid set forth in SEQ ID NO:68, a framework region 1 having the amino acid set forth in SEQ ID NO:76, or a framework region 1 having the amino acid set forth in SEQ ID NO:84. In another example, an Fab or scFv can be designed to include (a) the three CDRs set forth in FIG. 13, (b) the framework regions set forth in FIG. 2, 3, 4, 5, 6, 7, 8, 9, 10, 11, 12, or 13, and (c) a light chain variable domain.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:96. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:96. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:96.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain that includes an amino acid sequence having at least 90 percent identity to the amino acid sequence set forth in SEQ ID NO:96, provided that the heavy chain variable domain includes the amino acid sequences set forth in SEQ ID NOs:89, 90, and 91. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:96, provided that the heavy chain variable domain includes the amino acid sequences set forth in SEQ ID NOs:89, 90, and 91.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain having the amino acid sequence set forth in SEQ ID NO:96 or the amino acid set forth in SEQ ID NO:96 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a mesothelin polypeptide (e.g., a human mesothelin polypeptide) and can include a heavy chain variable domain having the amino acid sequence set forth in SEQ ID NO:96 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:89, 90, and 91.

In some cases, a binder (e.g., an antibody, antigen binding fragment, antibody domain, a CAR, a cell engager, and/or an ADC) provided herein having the ability to bind to a mesothelin polypeptide (e.g., a human mesothelin polypeptide) can include a heavy chain variable domain comprising (i) a CDR1 that comprises, consists essentially of, or consists of the amino acid sequence set forth in SEQ ID NO:89, (ii) a CDR2 that comprises, consists essentially of, or consists of the amino acid sequence set forth in SEQ ID NO:90, and (iii) a CDR3 that comprises, consists essentially of, or consists of the amino acid sequence set forth in SEQ ID NO:91. As used herein, a “CDR1 that consists essentially of the amino acid sequence set forth in SEQ ID NO:89” is a CDR1 that has zero, one, or two amino acid substitutions within SEQ ID NO:89, that has zero, one, two, three, four, or five amino acid residues directly preceding SEQ ID NO:89, and/or that has zero, one, two, three, four, or five amino acid residues directly following SEQ ID NO:89, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR1 that consists essentially of the amino acid sequence set forth in SEQ ID NO:89 include, without limitation, those set forth in Table 34.

TABLE 34
Exemplary CDR1s that consist essentially
of the amino acid sequence set forth
in SEQ ID NO: 89.

| Sequence | SEQ ID NO: |
|---|---|
| GYTFTGYY | 501 |
| GYTFTSYA | 502 |
| GYTFTSYG | 503 |
| GFTFTSSA | 504 |
| GFTFSSYW | 505 |
| GFTFDDYA | 506 |
| GFTFSSYA | 507 |
| GFTFSSYG | 508 |
| GFTFSSSA | 509 |
| GFTFSDHY | 510 |

As used herein, a “CDR2 that consists essentially of the amino acid sequence set forth in SEQ ID NO:90” is a CDR2 that has zero, one, or two amino acid substitutions within SEQ ID NO:90, that has zero, one, two, three, four, or five amino acid residues directly preceding SEQ ID NO:90, and/or that has zero, one, two, three, four, or five amino acid residues directly following SEQ ID NO:90, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR2 that consists essentially of the amino acid sequence set forth in SEQ ID NO:90 include, without limitation, those set forth in Table 35.

TABLE 35
Exemplary CDR2s that consist essentially
of the amino acid sequence set forth
in SEQ ID NO: 90.

| Sequence | SEQ ID NO: |
|---|---|
| INPNSGGT | 511 |
| ISAYNGNT | 512 |
| ISSSGST | 513 |
| IGTAGDT | 514 |
| ISSSSSYI | 515 |
| ISGSGGST | 516 |
| ISYDGSNK | 517 |
| IYSGGST | 518 |
| IYHSGST | 519 |
| ISGSGGST | 520 |

As used herein, a “CDR3 that consists essentially of the amino acid sequence set forth in SEQ ID NO:91” is a CDR3 that has zero, one, or two amino acid substitutions within SEQ ID NO:91, that has zero, one, two, three, four, or five amino acid residues directly preceding SEQ ID NO:91, and/or that has zero, one, two, three, four, or five amino acid residues directly following SEQ ID NO:91, provided that the binder (e.g., an antibody, antigen binding fragment, antibody domain, a CAR, a cell engager, and/or an ADC) maintains its basic ability to bind to a mesothelin polypeptide (e.g., a human mesothelin polypeptide). Examples of a CDR3 that consists essentially of the amino acid sequence set forth in SEQ ID NO:91 include, without limitation, those set forth in Table 36.

TABLE 36
Exemplary CDR3s that consist essentially
of the amino acid sequence set forth
in SEQ ID NO: 91.

| Sequence | SEQ ID NO: |
|---|---|
| ARRASGWALV | 521 |
| ATASGWALV | 522 |
| AAASGWALV | 523 |
| VRASGWALV | 524 |
| AHRASGWALV | 525 |
| ARIASGWALV | 526 |
| AKDASGWALV | 527 |
| TTASGWALV | 528 |
| SRASGWALV | 529 |
| AKASGWALV | 530 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:1-3; SEQ ID NOs:9-11; SEQ ID NOs:17-19; SEQ ID NOs:25-27; SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; or SEQ ID NOs:89-91 can be directly connected to a light chain variable domain via a linker sequence. Examples of linker sequences that can be used to connect a heavy chain variable domain and a light chain variable domain to create a scFv include, without limitation, those linkers set forth in FIG. 15.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a mesothelin polypeptide (e.g., a human mesothelin polypeptide) are further described in Table 37.

TABLE 37
Representative number of binders.

| | SEQ ID NOs of Heavy | SEQ ID NOs of Heavy | SEQ ID NO of |
|---|---|---|---|
| Clone # | Chain Variable Domain | Chain Variable Domain | Heavy Chain |
| (Antibody type) | CDRs | Framework Regions | Variable Domain |
| #1 (VH domain) | 1, 2, 3 | 4, 5, 6, 7 | 8 |
| #2 (VH domain) | 9, 10, 11 | 12, 13, 14, 15 | 16 |
| #3 (VH domain) | 17, 18, 19 | 20, 21, 22, 23 | 24 |
| #4 (VH domain) | 25, 26, 27 | 28, 29, 30, 31 | 32 |
| #5 (VH domain) | 33, 34, 35 | 36, 37, 38, 39 | 40 |
| #6 (VH domain) | 41, 42, 43 | 44, 45, 46, 47 | 48 |
| #7 (VH domain) | 49, 50, 51 | 52, 53, 54, 55 | 56 |
| #8 (VH domain) | 57, 58, 59 | 60, 61, 62, 63 | 64 |
| #9 (VH domain) | 65, 66, 67 | 68, 69, 70, 71 | 72 |
| #10 (VH domain) | 73, 74, 75 | 76, 77, 78, 79 | 80 |
| #11 (VH domain) | 81, 82, 83 | 84, 85, 86, 87 | 88 |
| #13 (VH domain) | 89, 90, 91 | 92, 93, 94, 95 | 96 |

Table 38 includes an alternative designation that can be used to refer to each of Clones #1-#12.

TABLE 38
Alternative nomenclature for Clones #1-#12.

| Clone # | Alternative names |
|---|---|
| 1 | ab1 |
| 2 | ab2 |
| 3 | ab3 |
| 4 | ab4 |
| 5 | ab5 |
| 6 | ab6 |
| 7 | ab7 |
| 8 | ab8 |
| 9 | ab9 |
| 10 | ab10 |
| 11 | ab11 |
| 12 | ab12 |

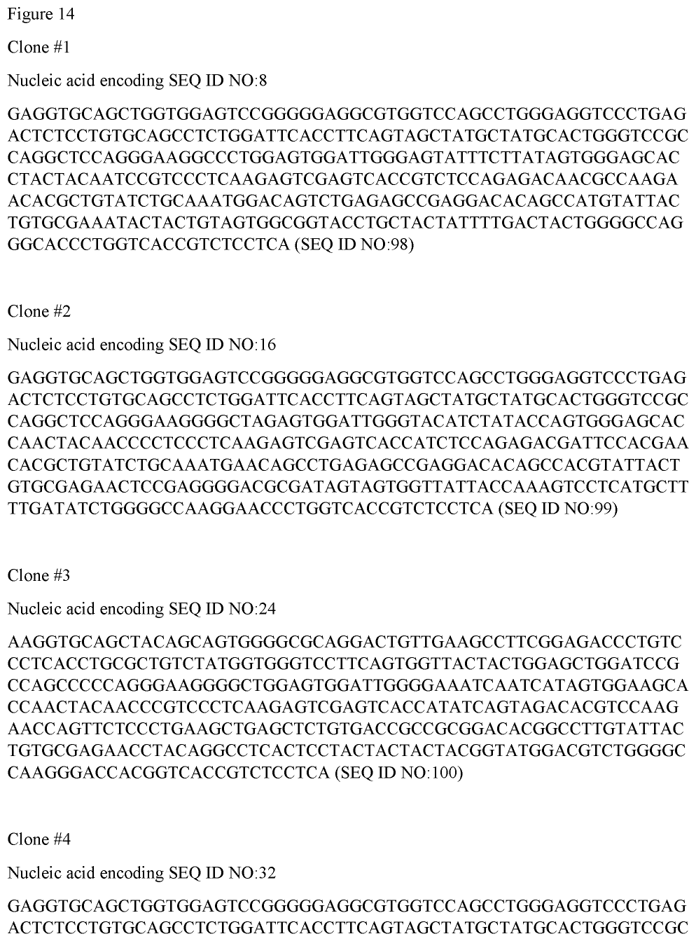

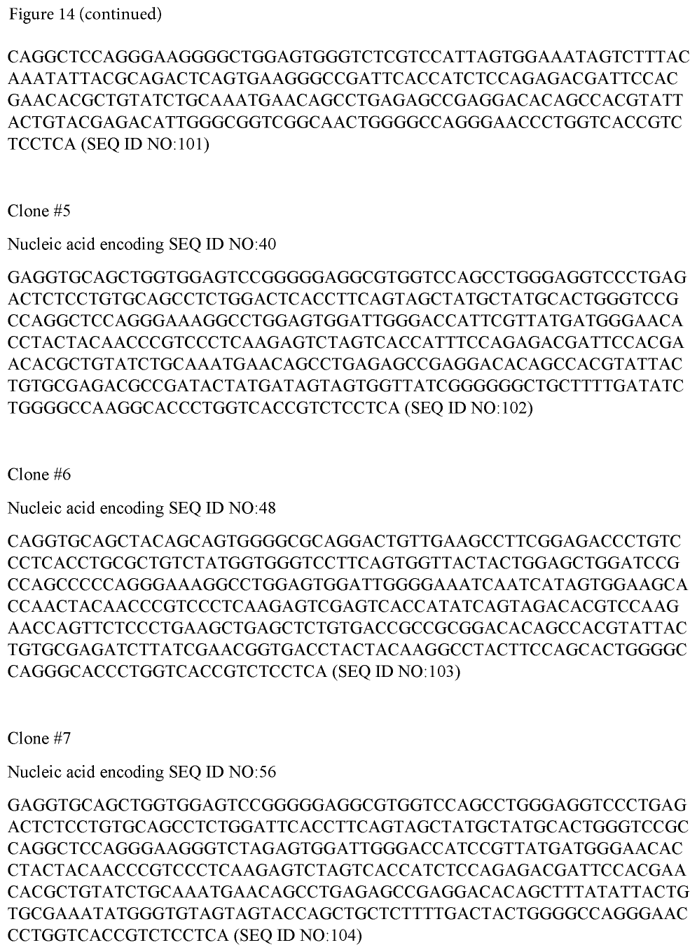

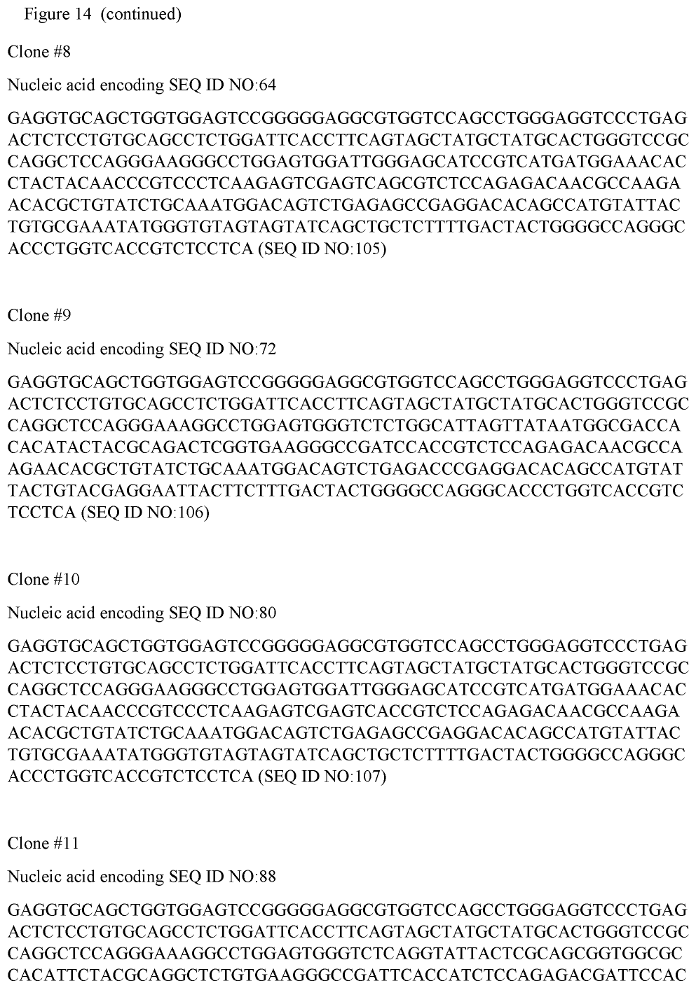

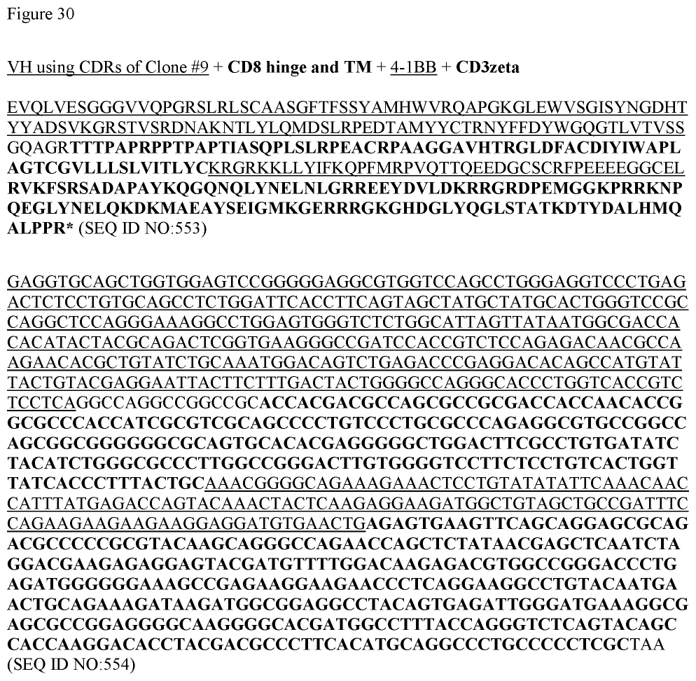

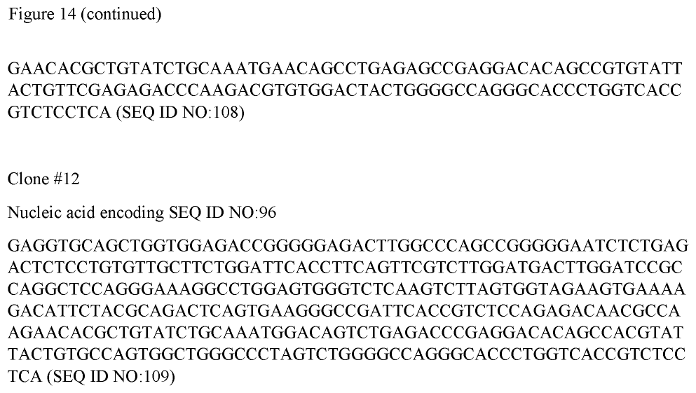

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. FIG. 14 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae/casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a mesothelin polypeptide (e.g., a human mesothelin polypeptide). In some cases, a bispecific binder provided herein can be designed to bind to two different epitopes of the same mesothelin polypeptide (e.g., a human mesothelin polypeptide). In some cases, a bispecific binder provided herein can bind to a mesothelin polypeptide (e.g., a human mesothelin polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a mesothelin polypeptide (e.g., a human mesothelin polypeptide) and (b) at least one drug covalently linked directly or indirectly to that antigen binding domain. Any appropriate binder (e.g., an antibody, antigen binding fragment, and/or antibody domain) provided herein and having the ability to bind to a mesothelin polypeptide (e.g., a human mesothelin polypeptide) can be used as an antigen binding domain to make an ADC described herein. For example, any of the binders set forth in Table 37 can be used to make an ADC having the ability to bind to a mesothelin polypeptide (e.g., a human mesothelin polypeptide). Examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to a mesothelin polypeptide (e.g., a human mesothelin polypeptide) to form an ADC provided herein. For example, cleavable or non-cleavable ADC linkers can be used to covalently attach one or more drugs to an antigen binding domain having the ability to bind to a mesothelin polypeptide (e.g., a human mesothelin polypeptide) to form an ADC provided herein. Examples of ADC linkers can be used to covalently attach one or more drugs to an antigen binding domain having the ability to bind to a mesothelin polypeptide (e.g., a human mesothelin polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a VH domain set forth in FIG. 2, 3, 4, 5, 6, 7, 8, 9, 10, 11, 12, or 13. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, CMV promotors (e.g., an immediate early CMV promotor), vaccinia virus promotors, and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, N Y (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokaryotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, N Y (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a mesothelin polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a mesothelin polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a mesothelin polypeptide (e.g., a human mesothelin polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imatinib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a mesothelin polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a mesothelin polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, mesothelioma cancer, ovarian cancer, pancreatic cancer (e.g., pancreatic adenocarcinoma), lung cancer (e.g., lung adenocarcinoma), and cholangiocarcinoma. In some cases, a mammal (e.g., a human) having a mesothelin⁺ cancer (e.g., a mesothelin⁺ mesothelioma cancer, mesothelin⁺ ovarian cancer, mesothelin⁺ pancreatic cancer (e.g., mesothelin⁺ pancreatic adenocarcinoma), mesothelin⁺ lung cancer (e.g., mesothelin⁺ lung adenocarcinoma), or mesothelin⁺ cholangiocarcinoma) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a mesothelin polypeptide (e.g., a human mesothelin polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a mesothelin polypeptide (e.g., a human mesothelin polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a mesothelin polypeptide (e.g., a human mesothelin polypeptide). In some cases, a binder (e.g., an antibody, antigen binding fragment, and/or antibody domain) provided herein can be immobilized, e.g., on a support, and retention of a mesothelin polypeptide (e.g., a human mesothelin polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a mesothelin polypeptide (e.g., a human mesothelin polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P ³³P, ¹²⁵I, ³H, ¹⁴C and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human Mesothelin Polypeptide

Large phage displayed antibody domain libraries were panned and screened to identify monoclonal antibody domains that bind to a human mesothelin polypeptide. To identify such monoclonal antibody domains, a human mesothelin polypeptide set forth in FIG. 1 was fused to the Fc region of human IgG1 at the C-terminus of the mesothelin sequence, and the mesothelin-Fc polypeptide was used for panning of human VH domain phage-displayed libraries. Twelve VH domains (Clones: #1, #2, #3, #4, #5, #6, #7, #8, #9, #10, #11, and #12; FIGS. 2-13) were identified.

Binding affinity and specificity to a human mesothelin polypeptide were tested using an ELISA and SPR (Blitz). Clones #1-#12 exhibited high affinity binding having EC 50 values of 1000 nM, 1.6 nM, 38 nM, 1.7 nM, 298 nM, 1000 nM, 16.4 nM, 6.5 nM, 944 nM, 107 nM, 98 nM, and 1.3 nM, respectively. All the clones bound to 293F cells displaying human mesothelin, but did not bind to 293F cells lacking human mesothelin, demonstrating that they can bind to mesothelin present on cells. Clone #4 and Clone #12 cross-reacted with macaque mesothelin.

### Example 2—Designing CARs from Binders Having the Ability to Bind to a Human Mesothelin Polypeptide

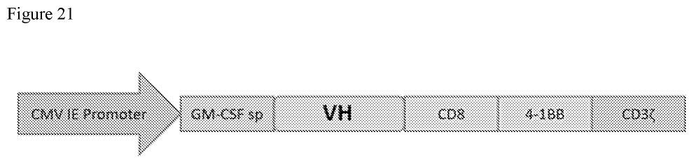

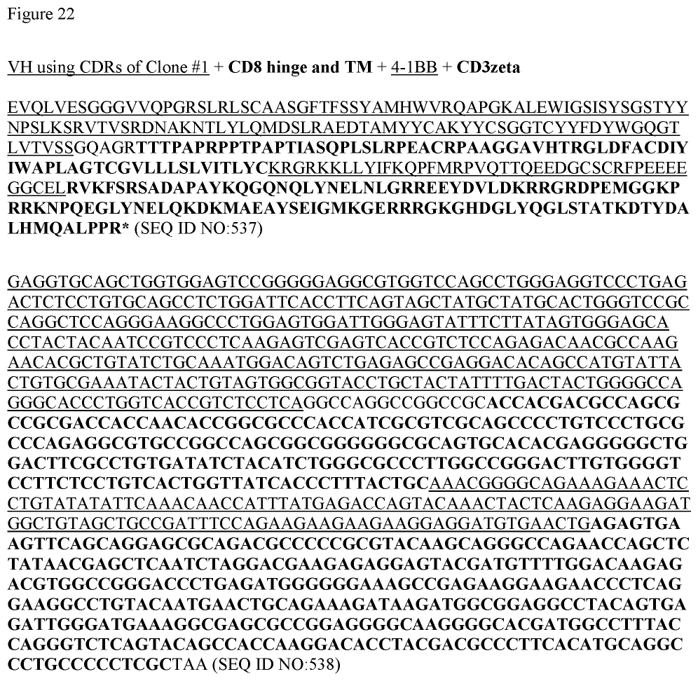

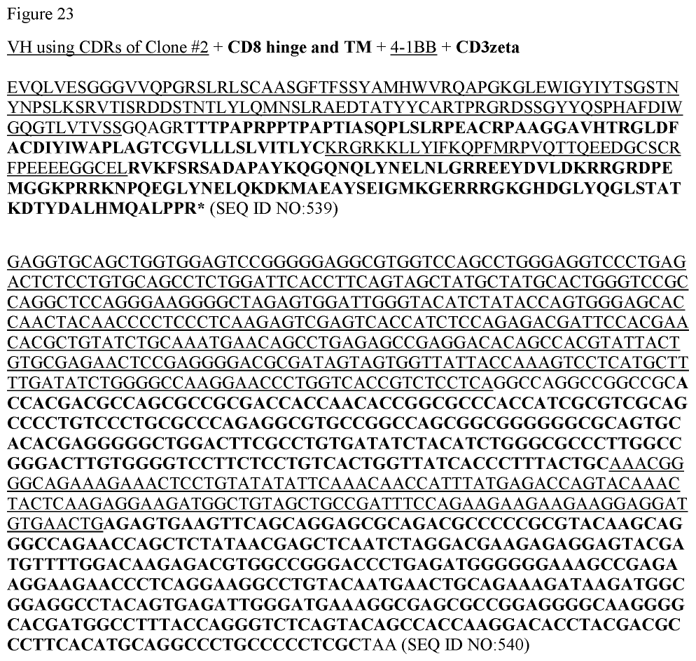

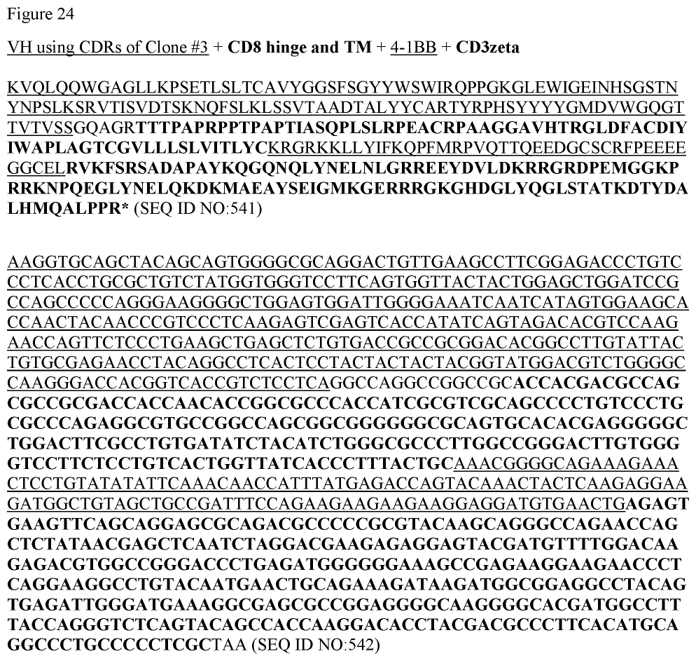

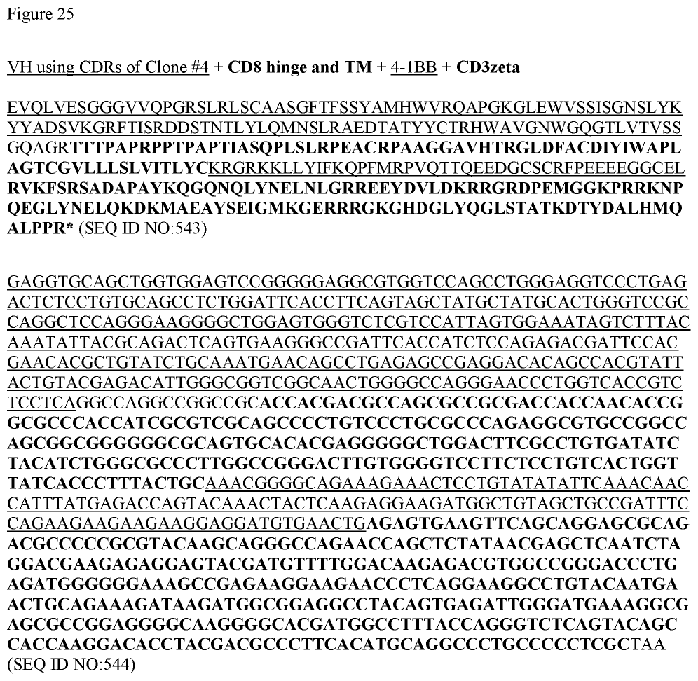

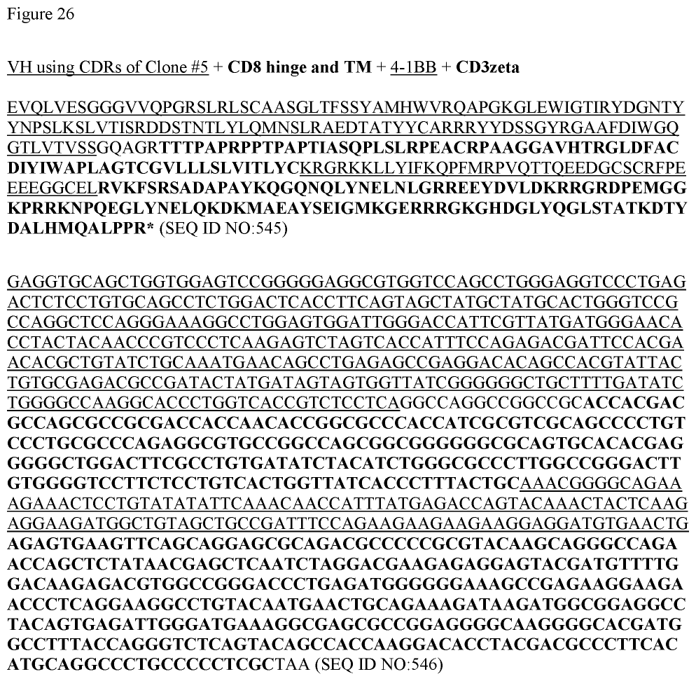

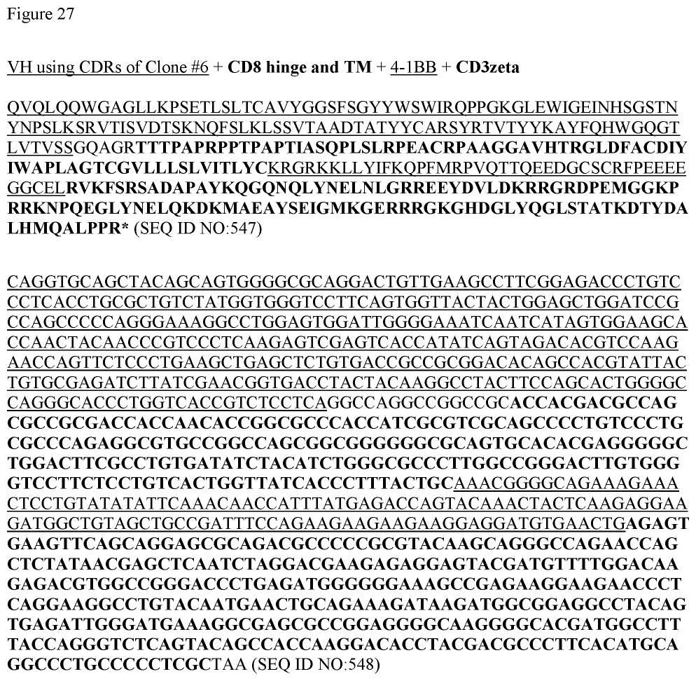

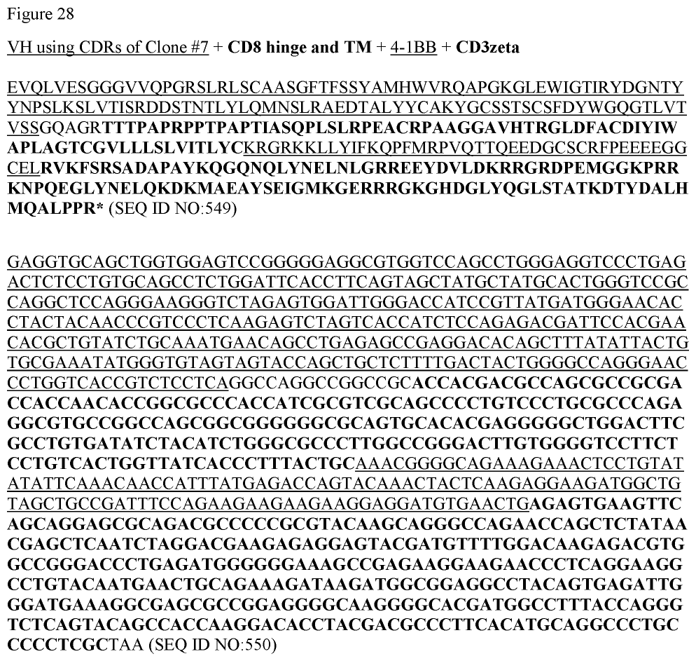

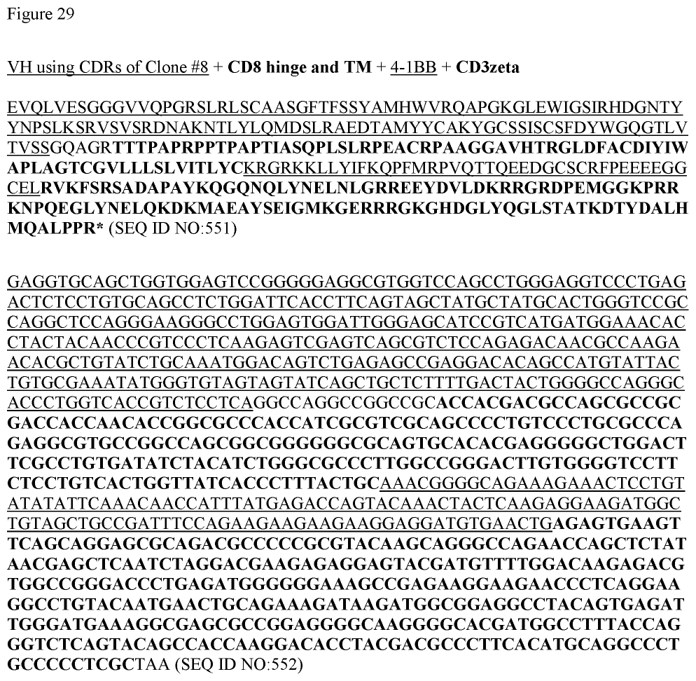

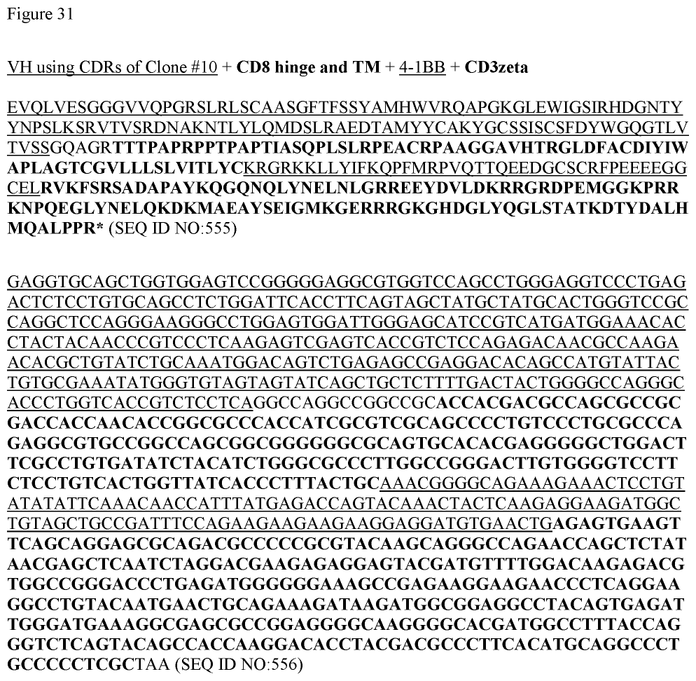

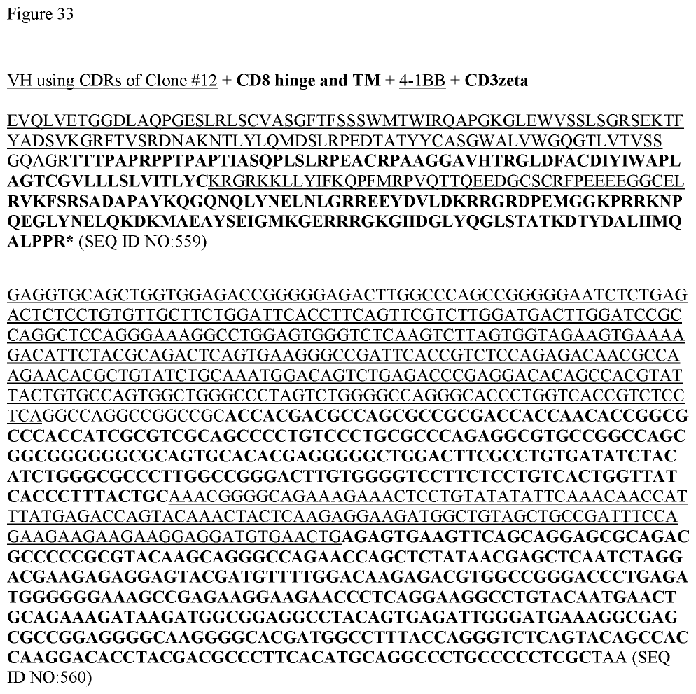

The VH domains of Clones #1-12 were used to create vectors designed to express CARs having the ability to bind to mesothelin polypeptides. See, e.g., FIG. 21. The amino acid sequence of CAR #1-CAR #12 and the nucleic acid sequences encoding those CARs are set forth in FIGS. 22-33, respectively. The vectors encoding CAR #1-#12 were individually transfected into T cells that were then used in a killing assay as effector cells. Briefly, CAR-T cells were incubated with 10,000 target cells (AsPC-1 cancer cells and HCl H2452 cancer cells) at a ratio of 20:1; 10:1; 5:1; or 2.5:1 in 96 well plates (for 8 hours and 24 hours, respectively). Untransduced human T cells (Mock) were used as a negative control. A maximum LDH release control was used where 2 μL of 10% Triton X-100 per 100 μL was added to target cell only wells for 10-15 minutes before collecting the samples for LDH detection. LDH release was detected using LDH-Glo™ Cytotoxicity Assay kit (Promega J2380) following the manufacturer's instructions. The percent cytotoxicity was calculated using the following formula: % Lysis=100×(Experimental LDH Release—Medium Background)/(Maximum LDH Release Control—Medium Background).

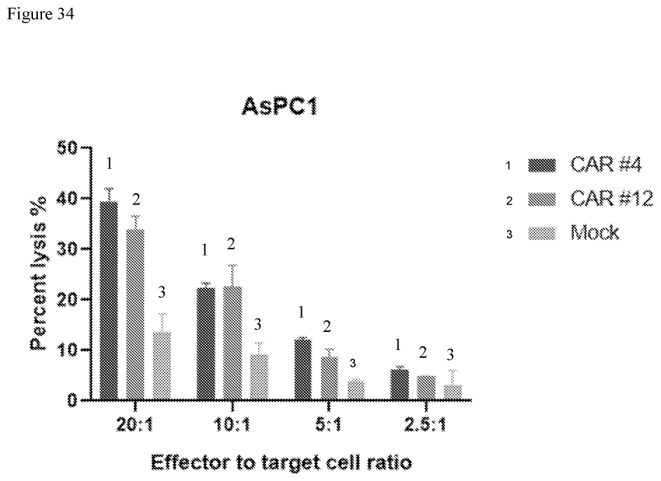

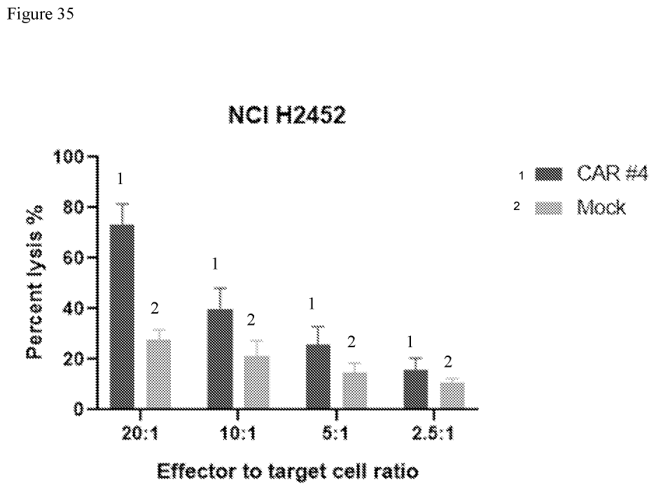

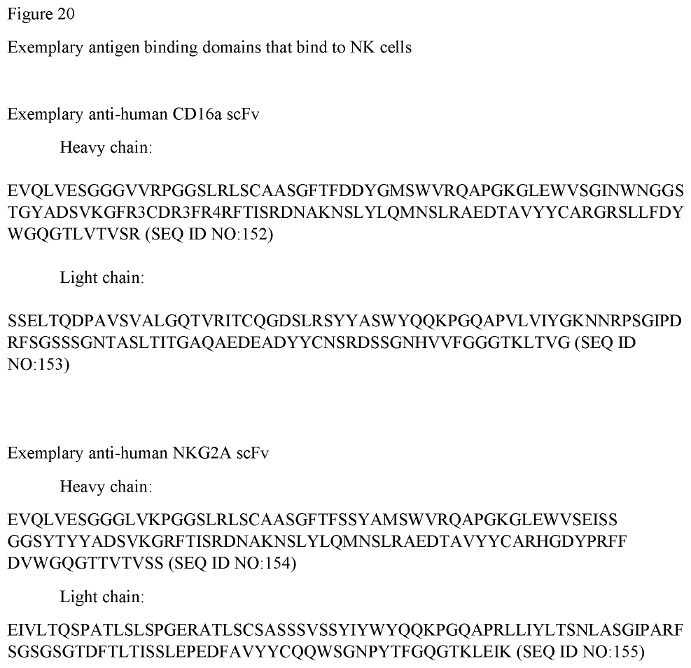

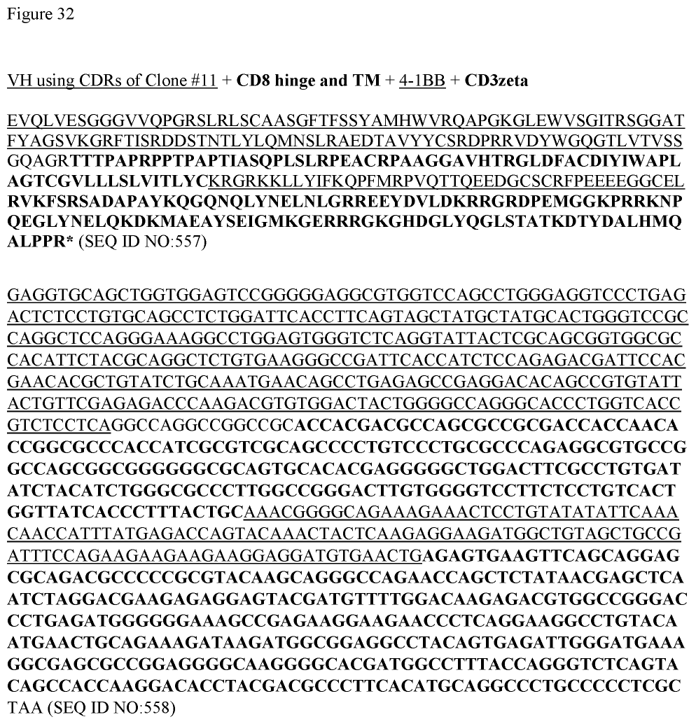

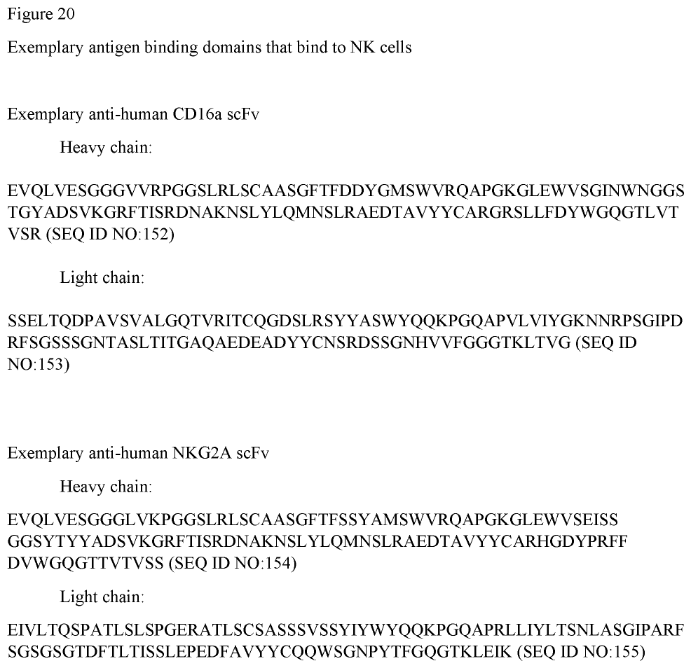

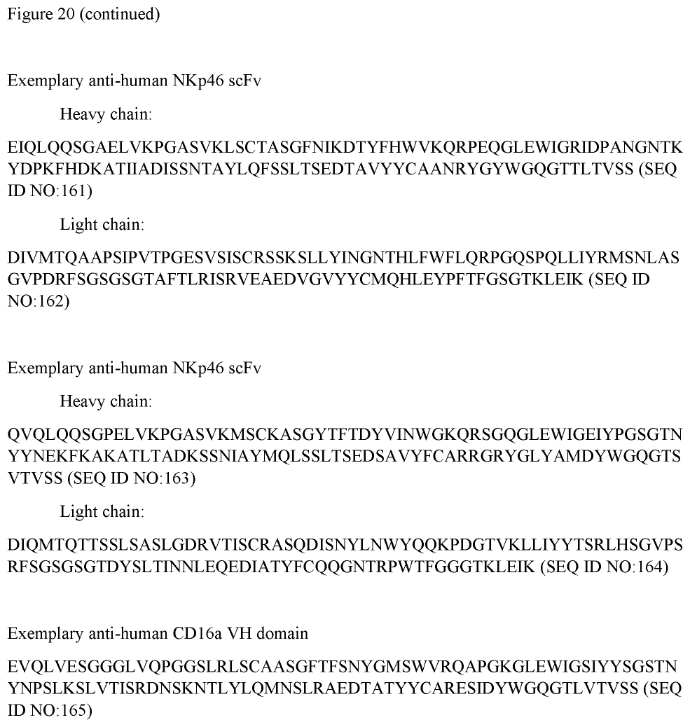

AsPC1 cell lysis was observed for T cells expressing CAR #4 and CAR #12 after 8 hours of incubation (FIG. 34). NCI H2452 cell lysis was observed for T cells expressing CAR #4 after 24 hours of incubation (FIG. 35). These results demonstrate that CARs having the ability to bind to mesothelin polypeptides can be designed using the CDRs and/or binders provided herein.

### Example 3—Other Embodiments

Embodiment 1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions);
(ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions);
(iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions);
(iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(v) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions);
(vi) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(vii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(viii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(ix) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(x) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(xi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); or
(xii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions).
   Embodiment 2. The antibody of embodiment 1, wherein said antibody comprises the ability to bind to SEQ ID NO:97 or SEQ ID NO:531.
   Embodiment 3. The antibody of any one of embodiments 1-2, wherein said antibody comprises said heavy chain variable domain or region of said (i).
   Embodiment 4. The antibody of embodiment 3, wherein said heavy chain variable domain or region comprises an amino acid sequence having at least 90 percent identity to the amino acid sequence set forth in SEQ ID NO:8.
   Embodiment 5. The antibody of any one of embodiments 1-2, wherein said antibody comprises said heavy chain variable domain or region of said (ii).
   Embodiment 6. The antibody of embodiment 5, wherein said heavy chain variable domain or region comprises an amino acid sequence having at least 90 percent identity to the amino acid sequence set forth in SEQ ID NO:16.
   Embodiment 7. The antibody of any one of embodiments 1-2, wherein said antibody comprises said heavy chain variable domain or region of said (iii).
   Embodiment 8. The antibody of embodiment 7, wherein said heavy chain variable domain or region comprises an amino acid sequence having at least 90 percent identity to the amino acid sequence set forth in SEQ ID NO:24.
   Embodiment 9. The antibody of any one of embodiments 1-2, wherein said antibody comprises said heavy chain variable domain or region of said (iv).
   Embodiment 10. The antibody of embodiment 9, wherein said heavy chain variable domain or region comprises an amino acid sequence having at least 90 percent identity to the amino acid sequence set forth in SEQ ID NO:32.
   Embodiment 11. The antibody of any one of embodiments 1-2, wherein said antibody comprises said heavy chain variable domain or region of said (v).
   Embodiment 12. The antibody of embodiment 11, wherein said heavy chain variable domain or region comprises an amino acid sequence having at least 90 percent identity to the amino acid sequence set forth in SEQ ID NO:40.
   Embodiment 13. The antibody of any one of embodiments 1-2, wherein said antibody comprises said heavy chain variable domain or region of said (vi).
   Embodiment 14. The antibody of embodiment 13, wherein said heavy chain variable domain or region comprises an amino acid sequence having at least 90 percent identity to the amino acid sequence set forth in SEQ ID NO:48.
   Embodiment 15. The antibody of any one of embodiments 1-2, wherein said antibody comprises said heavy chain variable domain or region of said (vii).
   Embodiment 16. The antibody of embodiment 15, wherein said heavy chain variable domain or region comprises an amino acid sequence having at least 90 percent identity to the amino acid sequence set forth in SEQ ID NO:56.
   Embodiment 17. The antibody of any one of embodiments 1-2, wherein said antibody comprises said heavy chain variable domain or region of said (viii).
   Embodiment 18. The antibody of embodiment 17, wherein said heavy chain variable domain or region comprises an amino acid sequence having at least 90 percent identity to the amino acid sequence set forth in SEQ ID NO:64.
   Embodiment 19. The antibody of any one of embodiments 1-2, wherein said antibody comprises said heavy chain variable domain or region of said (ix).
   Embodiment 20. The antibody of embodiment 20, wherein said heavy chain variable domain or region comprises an amino acid sequence having at least 90 percent identity to the amino acid sequence set forth in SEQ ID NO:72.
   Embodiment 21. The antibody of any one of embodiments 1-2, wherein said antibody comprises said heavy chain variable domain or region of said (x).
   Embodiment 22. The antibody of embodiment 21, wherein said heavy chain variable domain or region comprises an amino acid sequence having at least 90 percent identity to the amino acid sequence set forth in SEQ ID NO:80.
   Embodiment 23. The antibody of any one of embodiments 1-2, wherein said antibody comprises said heavy chain variable domain or region of said (xi).
   Embodiment 24. The antibody of embodiment 23, wherein said heavy chain variable domain or region comprises an amino acid sequence having at least 90 percent identity to the amino acid sequence set forth in SEQ ID NO:88.
   Embodiment 25. The antibody of any one of embodiments 1-2, wherein said antibody comprises said heavy chain variable domain or region of said (xii).
   Embodiment 26. The antibody of embodiment 25, wherein said heavy chain variable domain or region comprises an amino acid sequence having at least 90 percent identity to the amino acid sequence set forth in SEQ ID NO:96.
   Embodiment 27. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions);
(ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions);
(iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions);
(iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(v) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions);
(vi) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(vii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(viii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(ix) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(x) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(xi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); or
(xii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions).
   Embodiment 28. The antigen binding fragment of embodiment 27, wherein said antigen binding fragment comprises the ability to bind to SEQ ID NO:97 or SEQ ID NO:531.
   Embodiment 29. The antigen binding fragment of any one of embodiments 27-28, wherein said antigen binding fragment comprises said heavy chain variable domain or region of said (i).
   Embodiment 30. The antigen binding fragment of embodiment 29, wherein said heavy chain variable domain or region comprises an amino acid sequence having at least percent identity to the amino acid sequence set forth in SEQ ID NO:8.
   Embodiment 31. The antigen binding fragment of any one of embodiments 27-28, wherein said antigen binding fragment comprises said heavy chain variable domain or region of said (ii).
   Embodiment 32. The antigen binding fragment of embodiment 31, wherein said heavy chain variable domain or region comprises an amino acid sequence having at least percent identity to the amino acid sequence set forth in SEQ ID NO:16.
   Embodiment 33. The antigen binding fragment of any one of embodiments 27-28, wherein said antigen binding fragment comprises said heavy chain variable domain or region of said (iii).
   Embodiment 34. The antigen binding fragment of embodiment 33, wherein said heavy chain variable domain or region comprises an amino acid sequence having at least 90 percent identity to the amino acid sequence set forth in SEQ ID NO:24.
   Embodiment 35. The antigen binding fragment of any one of embodiments 27-28, wherein said antigen binding fragment comprises said heavy chain variable domain or region of said (iv).
   Embodiment 36. The antigen binding fragment of embodiment 35, wherein said heavy chain variable domain or region comprises an amino acid sequence having at least 90 percent identity to the amino acid sequence set forth in SEQ ID NO:32.
   Embodiment 37. The antigen binding fragment of any one of embodiments 27-28, wherein said antigen binding fragment comprises said heavy chain variable domain or region of said (v).
   Embodiment 38. The antigen binding fragment of embodiment 37, wherein said heavy chain variable domain or region comprises an amino acid sequence having at least percent identity to the amino acid sequence set forth in SEQ ID NO:40.
   Embodiment 39. The antigen binding fragment of any one of embodiments 27-28, wherein said antigen binding fragment comprises said heavy chain variable domain or region of said (vi).
   Embodiment 40. The antigen binding fragment of embodiment 39, wherein said heavy chain variable domain or region comprises an amino acid sequence having at least 90 percent identity to the amino acid sequence set forth in SEQ ID NO:48.
   Embodiment 41. The antigen binding fragment of any one of embodiments 27-28, wherein said antigen binding fragment comprises said heavy chain variable domain or region of said (vii).
   Embodiment 42. The antigen binding fragment of embodiment 41, wherein said heavy chain variable domain or region comprises an amino acid sequence having at least 90 percent identity to the amino acid sequence set forth in SEQ ID NO:56.
   Embodiment 43. The antigen binding fragment of any one of embodiments 27-28, wherein said antigen binding fragment comprises said heavy chain variable domain or region of said (viii).
   Embodiment 44. The antigen binding fragment of embodiment 43, wherein said heavy chain variable domain or region comprises an amino acid sequence having at least percent identity to the amino acid sequence set forth in SEQ ID NO:64.
   Embodiment 45. The antigen binding fragment of any one of embodiments 27-28, wherein said antigen binding fragment comprises said heavy chain variable domain or region of said (ix).
   Embodiment 46. The antigen binding fragment of embodiment 45, wherein said heavy chain variable domain or region comprises an amino acid sequence having at least percent identity to the amino acid sequence set forth in SEQ ID NO:72.
   Embodiment 47. The antigen binding fragment of any one of embodiments 27-28, wherein said antigen binding fragment comprises said heavy chain variable domain or region of said (x).
   Embodiment 48. The antigen binding fragment of embodiment 47, wherein said heavy chain variable domain or region comprises an amino acid sequence having at least 90 percent identity to the amino acid sequence set forth in SEQ ID NO:80.
   Embodiment 49. The antigen binding fragment of any one of embodiments 27-28, wherein said antigen binding fragment comprises said heavy chain variable domain or region of said (xi).
   Embodiment 50. The antigen binding fragment of embodiment 49, wherein said heavy chain variable domain or region comprises an amino acid sequence having at least 90 percent identity to the amino acid sequence set forth in SEQ ID NO:88.
   Embodiment 51. The antigen binding fragment of any one of embodiments 27-28, wherein said antigen binding fragment comprises said heavy chain variable domain or region of said (xii).
   Embodiment 52. The antigen binding fragment of embodiment 51, wherein said heavy chain variable domain or region comprises an amino acid sequence having at least percent identity to the amino acid sequence set forth in SEQ ID NO:96.
   Embodiment 53. The antibody of any one of embodiments 1-26, wherein said antibody is a monoclonal antibody.
   Embodiment 54. The antibody of any one of embodiments 1-26 and 53, wherein said antibody is an scFv antibody.
   Embodiment 55. The antigen binding fragment of any one of embodiments 27-52, wherein said antigen binding fragment is monoclonal.
   Embodiment 56. The antigen binding fragment of any one of embodiments 27-52 and 55, wherein said antigen binding fragment is an Fab.
   Embodiment 57. An antibody domain comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions);
(ii) a heavy chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:1 with one, two, or three amino acid additions, deletions, or substitutions);
(iii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions);
(iv) a heavy chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(v) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions);
(vi) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(vii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(viii) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(ix) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(x) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(xi) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); or
(xii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions).
   Embodiment 58. The antibody domain of embodiment 57, wherein said antibody domain comprises the ability to bind to SEQ ID NO:97 or SEQ ID NO:531.
   Embodiment 59. The antibody domain of any one of embodiments 57-58, wherein said antibody domain comprises said heavy chain variable domain or region of said (i).
   Embodiment 60. The antibody domain of embodiment 59, wherein said heavy chain variable domain or region comprises an amino acid sequence having at least 90 percent identity to the amino acid sequence set forth in SEQ ID NO:8.
   Embodiment 61. The antibody domain of any one of embodiments 57-58, wherein said antibody domain comprises said heavy chain variable domain or region of said (ii).
   Embodiment 62. The antibody domain of embodiment 59, wherein said heavy chain variable domain or region comprises an amino acid sequence having at least 90 percent identity to the amino acid sequence set forth in SEQ ID NO:16.
   Embodiment 63. The antibody domain of any one of embodiments 57-58, wherein said antibody domain comprises said heavy chain variable domain or region of said (iii).
   Embodiment 64. The antibody domain of embodiment 59, wherein said heavy chain variable domain or region comprises an amino acid sequence having at least 90 percent identity to the amino acid sequence set forth in SEQ ID NO:24.
   Embodiment 65. The antibody domain of any one of embodiments 57-58, wherein said antibody domain comprises said heavy chain variable domain or region of said (iv).
   Embodiment 66. The antibody domain of embodiment 59, wherein said heavy chain variable domain or region comprises an amino acid sequence having at least 90 percent identity to the amino acid sequence set forth in SEQ ID NO:32.
   Embodiment 67. The antibody domain of any one of embodiments 57-58, wherein said antibody domain comprises said heavy chain variable domain or region of said (v).
   Embodiment 68. The antibody domain of embodiment 59, wherein said heavy chain variable domain or region comprises an amino acid sequence having at least 90 percent identity to the amino acid sequence set forth in SEQ ID NO:40.
   Embodiment 69. The antibody domain of any one of embodiments 57-58, wherein said antibody domain comprises said heavy chain variable domain or region of said (vi).
   Embodiment 70. The antibody domain of embodiment 59, wherein said heavy chain variable domain or region comprises an amino acid sequence having at least 90 percent identity to the amino acid sequence set forth in SEQ ID NO:48.
   Embodiment 71. The antibody domain of any one of embodiments 57-58, wherein said antibody domain comprises said heavy chain variable domain or region of said (vii).
   Embodiment 72. The antibody domain of embodiment 59, wherein said heavy chain variable domain or region comprises an amino acid sequence having at least 90 percent identity to the amino acid sequence set forth in SEQ ID NO:56.
   Embodiment 73. The antibody domain of any one of embodiments 57-58, wherein said antibody domain comprises said heavy chain variable domain or region of said (viii).
   Embodiment 74. The antibody domain of embodiment 59, wherein said heavy chain variable domain or region comprises an amino acid sequence having at least 90 percent identity to the amino acid sequence set forth in SEQ ID NO:64.
   Embodiment 75. The antibody domain of any one of embodiments 57-58, wherein said antibody domain comprises said heavy chain variable domain or region of said (ix).
   Embodiment 76. The antibody domain of embodiment 59, wherein said heavy chain variable domain or region comprises an amino acid sequence having at least 90 percent identity to the amino acid sequence set forth in SEQ ID NO:72.
   Embodiment 77. The antibody domain of any one of embodiments 57-58, wherein said antibody domain comprises said heavy chain variable domain or region of said (x).
   Embodiment 78. The antibody domain of embodiment 59, wherein said heavy chain variable domain or region comprises an amino acid sequence having at least 90 percent identity to the amino acid sequence set forth in SEQ ID NO:80.
   Embodiment 79. The antibody domain of any one of embodiments 57-58, wherein said antibody domain comprises said heavy chain variable domain or region of said (xi).
   Embodiment 80. The antibody domain of embodiment 59, wherein said heavy chain variable domain or region comprises an amino acid sequence having at least 90 percent identity to the amino acid sequence set forth in SEQ ID NO:88.
   Embodiment 81. The antibody domain of any one of embodiments 57-58, wherein said antibody domain comprises said heavy chain variable domain or region of said (xii).
   Embodiment 82. The antibody domain of embodiment 59, wherein said heavy chain variable domain or region comprises an amino acid sequence having at least 90 percent identity to the amino acid sequence set forth in SEQ ID NO:96.
   Embodiment 83. The antibody domain of any one of embodiments 57-82, wherein said antibody domain is monoclonal.
   Embodiment 84. The antibody domain of any one of embodiments 57-83, wherein said antibody domain is a VH domain.
   Embodiment 85. A chimeric antigen receptor comprising an antigen binding domain, a hinge, a transmembrane domain, and one or more signaling domains, wherein said antigen binding domain comprises an antibody, an antigen-binding fragment, or an antibody domain of any one of embodiments 1-84.
   Embodiment 86. The chimeric antigen receptor of embodiment 85, wherein said antigen binding domain comprises a scFv having the ability to bind to a mesothelin polypeptide.
   Embodiment 87. The chimeric antigen receptor of embodiment 85, wherein said antigen binding domain comprises a VH domain having the ability to bind to a mesothelin polypeptide.
   Embodiment 88. The chimeric antigen receptor of any one of embodiments 85-87, wherein said hinge comprises a hinge set forth in FIG. 16.
   Embodiment 89. The chimeric antigen receptor of any one of embodiments 85-88, wherein said transmembrane domain comprises a transmembrane domain set forth in FIG. 17.
   Embodiment 90. The chimeric antigen receptor of any one of embodiments 85-89, wherein said chimeric antigen receptor comprises one or more signaling domains set forth in FIG. 18.
   Embodiment 91. A cell comprising a chimeric antigen receptor of any one of embodiments 85-90.
   Embodiment 92. The cell of embodiment 91, wherein said cell is a T cell, a stem cell, or an NK cell.
   Embodiment 93. A cell engager comprising a first antigen binding domain, a linker, and a second antigen binding domain, wherein said first antigen binding domain comprises an antibody, an antigen-binding fragment, or an antibody domain of any one of embodiments 1-84.
   Embodiment 94. The cell engager of embodiment 93, wherein said first antigen binding domain comprises a scFv having the ability to bind to a mesothelin polypeptide.
   Embodiment 95. The cell engager of embodiment 93, wherein said first antigen binding domain comprises a VH domain having the ability to bind to a mesothelin polypeptide.
   Embodiment 96. The cell engager of any one of embodiments 93-95, wherein said linker comprises a linker set forth in FIG. 15 or FIG. 16.
   Embodiment 97. The cell engager of any one of embodiments 93-96, wherein said second antigen binding domain binds to a polypeptide expressed on the surface of T cells.
   Embodiment 98. The cell engager of embodiment 97, wherein said polypeptide expressed on the surface of T cells is a CD3 polypeptide.
   Embodiment 99. The cell engager of embodiment 97, wherein said second antigen binding domain is an antigen binding domain set forth in FIG. 19.
   Embodiment 100. The cell engager of any one of embodiments 93-96, wherein said second antigen binding domain binds to a polypeptide expressed on the surface of NK cells.
   Embodiment 101. The cell engager of embodiment 100, wherein said polypeptide expressed on the surface of NK cells is a CD16a, NKG2A, NKG2D, NKp30, NKp44, or NKp46 polypeptide.
   Embodiment 102. The cell engager of embodiment 100, wherein said second antigen binding domain is an antigen binding domain set forth in FIG. 20.
   Embodiment 103. The cell engager of any one of embodiments 93-102, wherein said cell engager comprises a third antigen binding domain.
   Embodiment 104. The cell engager of embodiment 103, wherein said third antigen binding domain binds to a polypeptide expressed on the surface of NK cells.
   Embodiment 105. The cell engager of embodiment 104, wherein said polypeptide expressed on the surface of NK cells is a CD16a, NKG2A, NKG2D, NKp30, NKp44, or NKp46 polypeptide.
   Embodiment 106. The cell engager of embodiment 104, wherein said third antigen binding domain is an antigen binding domain set forth in FIG. 20.
   Embodiment 107. A nucleic acid comprising a nucleic acid sequence encoding at least part of said antibody, said antigen-binding fragment, or said antibody domain of any one of embodiments 1-84.
   Embodiment 108. The nucleic acid of embodiment 107, wherein said nucleic acid sequence encodes said heavy chain variable domain or region of any one of said (i)-(xii) of embodiment 1.
   Embodiment 109. The nucleic acid of any one of embodiments 107-108, wherein said nucleic acid is a viral vector.
   Embodiment 110. The nucleic acid of any one of embodiments 107-108, wherein said nucleic acid is a phagemid.
   Embodiment 111. A nucleic acid comprising a nucleic acid sequence encoding a chimeric antigen receptor of any one of embodiments 85-90 or a cell engager of any one of embodiments 93-106.
   Embodiment 112. The nucleic acid of embodiment 111, wherein said nucleic acid is a viral vector.
   Embodiment 113. The nucleic acid of embodiment 111, wherein said nucleic acid is a phagemid.
   Embodiment 114. A host cell comprising a nucleic acid of any one of embodiments 111-113.
   Embodiment 115. A host cell that expresses a chimeric antigen receptor of any one of embodiments 85-90 or a cell engager of any one of embodiments 93-106.
   Embodiment 116. The host cell of any one of embodiments 114-115, wherein said host cell is a T cell, stem cell, or NK cell.
   Embodiment 117. An antibody-drug conjugate (ADC) comprising an antigen binging domain covalently linked to a drug, wherein said antigen binging domain comprises an antibody, an antigen binding fragment, or an antibody domain of any one of embodiments 1-84.
   Embodiment 118. The ADC of embodiment 117, wherein said antigen binding domain comprises a scFv having the ability to bind to a mesothelin polypeptide.
   Embodiment 119. The ADC of embodiment 117, wherein said antigen binding domain comprises a VH domain having the ability to bind to a mesothelin polypeptide.
   Embodiment 120. The ADC of any one of embodiments 117-119, wherein said drug is selected from the group consisting of auristatins, mertansine, or pyrrolobenzodiazepine (PBD) dimers.
   Embodiment 121. A composition comprising an antibody, an antigen binding fragment, or an antibody domain of any one of embodiments 1-84.
   Embodiment 122. The composition of embodiment 121, wherein said composition comprises said antibody of any one of embodiments 1-26, 53, and 54.
   Embodiment 123. The composition of embodiment 121, wherein said composition comprises said antigen binding fragment of any one of embodiments 27-52, 55, and 56.
   Embodiment 124. The composition of embodiment 121, wherein said composition comprises said antibody domain of any one of embodiments 57-84.
   Embodiment 125. A composition comprising a cell engager of any one of embodiments 93-106.
   Embodiment 126. A composition comprising a cell of any one of embodiments 91, 92, and 114-116.
   Embodiment 127. A composition comprising an ADC of any one of embodiments 117-120.
   Embodiment 128. The composition of any one of embodiments 121-127, wherein said composition comprises a checkpoint inhibitor.
   Embodiment 129. The composition of embodiment 128, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.
   Embodiment 130. A method of treating a mammal having cancer, wherein said method comprises administering, to said mammal, a composition of any one of embodiments 121-129.
   Embodiment 131. The method of embodiment 130, wherein said mammal is a human.
   Embodiment 132. The method of any one of embodiments 130-131, wherein said cancer is a mesothelin⁺ cancer.
   Embodiment 133. The method of embodiment 133, wherein said mesothelin⁺ cancer is selected from the group consisting of mesothelin⁺ mesothelioma cancer, mesothelin⁺ ovarian cancer, mesothelin⁺ pancreatic cancer, mesothelin⁺ lung cancer and mesothelin⁺ cholangiocarcinoma.
   Embodiment 134. The method of any one of embodiments 130-133, wherein the number of cancer cells within said mammal is reduced following said administering step.
   Embodiment 135. A method of treating a mammal having cancer, wherein said method comprises:
(a) administering, to said mammal, said composition of any one of embodiments 121-127, and
(b) administering, to said mammal, a composition comprising a checkpoint inhibitor.
   Embodiment 136. The method of embodiment 135, wherein said mammal is a human.
   Embodiment 137. The method of any one of embodiments 135-136, wherein said cancer is a mesothelin⁺ cancer.
   Embodiment 138. The method of embodiment 137, wherein said mesothelin⁺ cancer is selected from the group consisting of mesothelin⁺ mesothelioma cancer, mesothelin⁺ ovarian cancer, mesothelin⁺ pancreatic cancer, mesothelin⁺ lung cancer and mesothelin⁺ cholangiocarcinoma.
   Embodiment 139. The method of any one of embodiments 135-138, wherein said checkpoint inhibitor is selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.
   Embodiment 140. The method of any one of embodiments 135-139, wherein the number of cancer cells within said mammal is reduced following said administering steps (a) and (b).
   Embodiment 141. A method for binding a binding molecule to a mesothelin polypeptide, wherein said method comprises contacting said mesothelin polypeptide with an antibody, an antigen binding fragment, or an antibody domain of any one of embodiments 1-84.
   Embodiment 142. The method of embodiment 141, wherein said contacting is performed in vitro.
   Embodiment 143. The method of embodiment 141, wherein said contacting is performed in vivo.
   Embodiment 144. The method of embodiment 143, wherein said contacting is performed within a mammal by administering said antibody, said antigen binding fragment, or said antibody domain to said mammal.
   Embodiment 145. The method of embodiment 144, wherein said mammal is a human.
   Embodiment 146. A method for binding a binding molecule to a mesothelin polypeptide, wherein said method comprises contacting said mesothelin polypeptide with a chimeric antigen receptor of any one of embodiments 85-90, a cell engager of any one of embodiments 93-106, or an ADC of any one of embodiments 117-120.
   Embodiment 147. The method of embodiment 146, wherein said contacting is performed in vitro.
   Embodiment 148. The method of embodiment 146, wherein said contacting is performed in vivo.
   Embodiment 149. The method of embodiment 148, wherein said contacting is performed within a mammal by administering said chimeric antigen receptor, said cell engager, or said ADC to said mammal.
   Embodiment 150. The method of embodiment 149, wherein said mammal is a human.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO:1 or SEQ ID NO:1 with one, two, or three amino acid additions, deletions, or substitutions, SEQ ID NO:2 or SEQ ID NO:2 with one, two, or three amino acid additions, deletions, or substitutions, and SEQ ID NO:3 or SEQ ID NO:3 with one, two, or three amino acid additions, deletions, or substitutions;
(ii) a heavy chain variable domain or region comprising the amino acid sequences set forth in SEQ ID NO:9 or SEQ ID NO:9 with one, two, or three amino acid additions, deletions, or substitutions, SEQ ID NO:10 or SEQ ID NO:10 with one, two, or three amino acid additions, deletions, or substitutions, and SEQ ID NO:11 or SEQ ID NO:1 with one, two, or three amino acid additions, deletions, or substitutions,
(iii) a heavy chain variable domain or region comprising the amino acid sequences set forth in SEQ ID NO:17 or SEQ ID NO:17 with one, two, or three amino acid additions, deletions, or substitutions, SEQ ID NO:18 or SEQ ID NO:18 with one, two, or three amino acid additions, deletions, or substitutions, and SEQ ID NO:19 or SEQ ID NO:19 with one, two, or three amino acid additions, deletions, or substitutions;
(iv) a heavy chain variable domain or region comprising the amino acid sequences set forth in SEQ ID NO:25 or SEQ ID NO:25 with one, two, or three amino acid additions, deletions, or substitutions, SEQ ID NO:26 or SEQ ID NO:26 with one, two, or three amino acid additions, deletions, or substitutions, and SEQ ID NO:27 or SEQ ID NO:27 with one, two, or three amino acid additions, deletions, or substitutions;
(v) a heavy chain variable domain or region comprising the amino acid sequences set forth in SEQ ID NO:33 or SEQ ID NO:33 with one, two, or three amino acid additions, deletions, or substitutions, SEQ ID NO:34 or SEQ ID NO:34 with one, two, or three amino acid additions, deletions, or substitutions, and SEQ ID NO:35 or SEQ ID NO:35 with one, two, or three amino acid additions, deletions, or substitutions;
(vi) a heavy chain variable domain or region comprising the amino acid sequences set forth in SEQ ID NO:41 or SEQ ID NO:41 with one, two, or three amino acid additions, deletions, or substitutions, SEQ ID NO:42 or SEQ ID NO:42 with one, two, or three amino acid additions, deletions, or substitutions, and SEQ ID NO:43 or SEQ ID NO:43 with one, two, or three amino acid additions, deletions, or substitutions;
(vii) a heavy chain variable domain or region comprising the amino acid sequences set forth in SEQ ID NO:49 or SEQ ID NO:49 with one, two, or three amino acid additions, deletions, or substitutions, SEQ ID NO:50 or SEQ ID NO:50 with one, two, or three amino acid additions, deletions, or substitutions, and SEQ ID NO:51 or SEQ ID NO:51 with one, two, or three amino acid additions, deletions, or substitutions;
(viii) a heavy chain variable domain or region comprising the amino acid sequences set forth in SEQ ID NO:57 or SEQ ID NO:57 with one, two, or three amino acid additions, deletions, or substitutions, SEQ ID NO:58 or SEQ ID NO:58 with one, two, or three amino acid additions, deletions, or substitutions, and SEQ ID NO:59 or SEQ ID NO:59 with one, two, or three amino acid additions, deletions, or substitutions;
(ix) a heavy chain variable domain or region comprising the amino acid sequences set forth in SEQ ID NO:65 or SEQ ID NO:65 with one, two, or three amino acid additions, deletions, or substitutions, SEQ ID NO:66 or SEQ ID NO:66 with one, two, or three amino acid additions, deletions, or substitutions, and SEQ ID NO:67 or SEQ ID NO:67 with one, two, or three amino acid additions, deletions, or substitutions;
(x) a heavy chain variable domain or region comprising the amino acid sequences set forth in SEQ ID NO:73 or SEQ ID NO:73 with one, two, or three amino acid additions, deletions, or substitutions, SEQ ID NO:74 or SEQ ID NO:74 with one, two, or three amino acid additions, deletions, or substitutions, and SEQ ID NO:75 or SEQ ID NO:75 with one, two, or three amino acid additions, deletions, or substitutions;
(xi) a heavy chain variable domain or region comprising the amino acid sequences set forth in SEQ ID NO:81 or SEQ ID NO:81 with one, two, or three amino acid additions, deletions, or substitutions, SEQ ID NO:82 or SEQ ID NO:82 with one, two, or three amino acid additions, deletions, or substitutions, and SEQ ID NO:83 or SEQ ID NO:83 with one, two, or three amino acid additions, deletions, or substitutions; or
(xii) a heavy chain variable domain or region comprising the amino acid sequences set forth in SEQ ID NO:89 or SEQ ID NO:89 with one, two, or three amino acid additions, deletions, or substitutions, SEQ ID NO:90 or SEQ ID NO:90 with one, two, or three amino acid additions, deletions, or substitutions, and SEQ ID NO:91 or SEQ ID NO:91 with one, two, or three amino acid additions, deletions, or substitutions.

2. (canceled)

3. An antigen binding fragment comprising:
(i) a heavy chain variable domain or region comprising the amino acid sequences set forth in SEQ ID NO:1 or SEQ ID NO:1 with one, two, or three amino acid additions, deletions, or substitutions, SEQ ID NO:2 or SEQ ID NO:2 with one, two, or three amino acid additions, deletions, or substitutions, and SEQ ID NO:3 or SEQ ID NO:3 with one, two, or three amino acid additions, deletions, or substitutions;
(ii) a heavy chain variable domain or region comprising the amino acid sequences set forth in SEQ ID NO:9 or SEQ ID NO:9 with one, two, or three amino acid additions, deletions, or substitutions, SEQ ID NO:10 or SEQ ID NO:10 with one, two, or three amino acid additions, deletions, or substitutions, and SEQ ID NO:11 or SEQ ID NO:1 with one, two, or three amino acid additions, deletions, or substitutions,
(iii) a heavy chain variable domain or region comprising the amino acid sequences set forth in SEQ ID NO:17 or SEQ ID NO:17 with one, two, or three amino acid additions, deletions, or substitutions, SEQ ID NO:18 or SEQ ID NO:18 with one, two, or three amino acid additions, deletions, or substitutions, and SEQ ID NO:19 or SEQ ID NO:19 with one, two, or three amino acid additions, deletions, or substitutions;
(iv) a heavy chain variable domain or region comprising the amino acid sequences set forth in SEQ ID NO:25 or SEQ ID NO:25 with one, two, or three amino acid additions, deletions, or substitutions, SEQ ID NO:26 or SEQ ID NO:26 with one, two, or three amino acid additions, deletions, or substitutions, and SEQ ID NO:27 or SEQ ID NO:27 with one, two, or three amino acid additions, deletions, or substitutions;
(v) a heavy chain variable domain or region comprising the amino acid sequences set forth in SEQ ID NO:33 or SEQ ID NO:33 with one, two, or three amino acid additions, deletions, or substitutions, SEQ ID NO:34 or SEQ ID NO:34 with one, two, or three amino acid additions, deletions, or substitutions, and SEQ ID NO:35 or SEQ ID NO:35 with one, two, or three amino acid additions, deletions, or substitutions;
(vi) a heavy chain variable domain or region comprising the amino acid sequences set forth in SEQ ID NO:41 or SEQ ID NO:41 with one, two, or three amino acid additions, deletions, or substitutions, SEQ ID NO:42 or SEQ ID NO:42 with one, two, or three amino acid additions, deletions, or substitutions, and SEQ ID NO:43 or SEQ ID NO:43 with one, two, or three amino acid additions, deletions, or substitutions;
(vii) a heavy chain variable domain or region comprising the amino acid sequences set forth in SEQ ID NO:49 or SEQ ID NO:49 with one, two, or three amino acid additions, deletions, or substitutions, SEQ ID NO:50 or SEQ ID NO:50 with one, two, or three amino acid additions, deletions, or substitutions, and SEQ ID NO:51 or SEQ ID NO:51 with one, two, or three amino acid additions, deletions, or substitutions;
(viii) a heavy chain variable domain or region comprising the amino acid sequences set forth in SEQ ID NO:57 or SEQ ID NO:57 with one, two, or three amino acid additions, deletions, or substitutions, SEQ ID NO:58 or SEQ ID NO:58 with one, two, or three amino acid additions, deletions, or substitutions, and SEQ ID NO:59 or SEQ ID NO:59 with one, two, or three amino acid additions, deletions, or substitutions;
(ix) a heavy chain variable domain or region comprising the amino acid sequences set forth in SEQ ID NO:65 or SEQ ID NO:65 with one, two, or three amino acid additions, deletions, or substitutions, SEQ ID NO:66 or SEQ ID NO:66 with one, two, or three amino acid additions, deletions, or substitutions, and SEQ ID NO:67 or SEQ ID NO:67 with one, two, or three amino acid additions, deletions, or substitutions;
(x) a heavy chain variable domain or region comprising the amino acid sequences set forth in SEQ ID NO:73 or SEQ ID NO:73 with one, two, or three amino acid additions, deletions, or substitutions, SEQ ID NO:74 or SEQ ID NO:74 with one, two, or three amino acid additions, deletions, or substitutions, and SEQ ID NO:75 or SEQ ID NO:75 with one, two, or three amino acid additions, deletions, or substitutions;
(xi) a heavy chain variable domain or region comprising the amino acid sequences set forth in SEQ ID NO:81 or SEQ ID NO:81 with one, two, or three amino acid additions, deletions, or substitutions, SEQ ID NO:82 or SEQ ID NO:82 with one, two, or three amino acid additions, deletions, or substitutions, and SEQ ID NO:83 or SEQ ID NO:83 with one, two, or three amino acid additions, deletions, or substitutions; or
(xii) a heavy chain variable domain or region comprising the amino acid sequences set forth in SEQ ID NO:89 or SEQ ID NO:89 with one, two, or three amino acid additions, deletions, or substitutions, SEQ ID NO:90 or SEQ ID NO:90 with one, two, or three amino acid additions, deletions, or substitutions, and SEQ ID NO:91 or SEQ ID NO:91 with one, two, or three amino acid additions, deletions, or substitutions.

4-6. (canceled)

7. An antibody domain comprising:
(i) a heavy chain variable domain or region comprising the amino acid sequences set forth in SEQ ID NO:1 or SEQ ID NO:1 with one, two, or three amino acid additions, deletions, or substitutions, SEQ ID NO:2 or SEQ ID NO:2 with one, two, or three amino acid additions, deletions, or substitutions, and SEQ ID NO:3 or SEQ ID NO:3 with one, two, or three amino acid additions, deletions, or substitutions;
(ii) a heavy chain variable domain or region comprising the amino acid sequences set forth in SEQ ID NO:9 or SEQ ID NO:9 with one, two, or three amino acid additions, deletions, or substitutions, SEQ ID NO:10 or SEQ ID NO:10 with one, two, or three amino acid additions, deletions, or substitutions, and SEQ ID NO:11 or SEQ ID NO:1 with one, two, or three amino acid additions, deletions, or substitutions,
(iii) a heavy chain variable domain or region comprising the amino acid sequences set forth in SEQ ID NO:17 or SEQ ID NO:17 with one, two, or three amino acid additions, deletions, or substitutions, SEQ ID NO:18 or SEQ ID NO:18 with one, two, or three amino acid additions, deletions, or substitutions, and SEQ ID NO:19 or SEQ ID NO:19 with one, two, or three amino acid additions, deletions, or substitutions;
(iv) a heavy chain variable domain or region comprising the amino acid sequences set forth in SEQ ID NO:25 or SEQ ID NO:25 with one, two, or three amino acid additions, deletions, or substitutions, SEQ ID NO:26 or SEQ ID NO:26 with one, two, or three amino acid additions, deletions, or substitutions, and SEQ ID NO:27 or SEQ ID NO:27 with one, two, or three amino acid additions, deletions, or substitutions;
(v) a heavy chain variable domain or region comprising the amino acid sequences set forth in SEQ ID NO:33 or SEQ ID NO:33 with one, two, or three amino acid additions, deletions, or substitutions, SEQ ID NO:34 or SEQ ID NO:34 with one, two, or three amino acid additions, deletions, or substitutions, and SEQ ID NO:35 or SEQ ID NO:35 with one, two, or three amino acid additions, deletions, or substitutions;
(vi) a heavy chain variable domain or region comprising the amino acid sequences set forth in SEQ ID NO:41 or SEQ ID NO:41 with one, two, or three amino acid additions, deletions, or substitutions, SEQ ID NO:42 or SEQ ID NO:42 with one, two, or three amino acid additions, deletions, or substitutions, and SEQ ID NO:43 or SEQ ID NO:43 with one, two, or three amino acid additions, deletions, or substitutions;
(vii) a heavy chain variable domain or region comprising the amino acid sequences set forth in SEQ ID NO:49 or SEQ ID NO:49 with one, two, or three amino acid additions, deletions, or substitutions, SEQ ID NO:50 or SEQ ID NO:50 with one, two, or three amino acid additions, deletions, or substitutions, and SEQ ID NO:51 or SEQ ID NO:51 with one, two, or three amino acid additions, deletions, or substitutions;
(viii) a heavy chain variable domain or region comprising the amino acid sequences set forth in SEQ ID NO:57 or SEQ ID NO:57 with one, two, or three amino acid additions, deletions, or substitutions, SEQ ID NO:58 or SEQ ID NO:58 with one, two, or three amino acid additions, deletions, or substitutions, and SEQ ID NO:59 or SEQ ID NO:59 with one, two, or three amino acid additions, deletions, or substitutions;
(ix) a heavy chain variable domain or region comprising the amino acid sequences set forth in SEQ ID NO:65 or SEQ ID NO:65 with one, two, or three amino acid additions, deletions, or substitutions, SEQ ID NO:66 or SEQ ID NO:66 with one, two, or three amino acid additions, deletions, or substitutions, and SEQ ID NO:67 or SEQ ID NO:67 with one, two, or three amino acid additions, deletions, or substitutions;
(x) a heavy chain variable domain or region comprising the amino acid sequences set forth in SEQ ID NO:73 or SEQ ID NO:73 with one, two, or three amino acid additions, deletions, or substitutions, SEQ ID NO:74 or SEQ ID NO:74 with one, two, or three amino acid additions, deletions, or substitutions, and SEQ ID NO:75 or SEQ ID NO:75 with one, two, or three amino acid additions, deletions, or substitutions;
(xi) a heavy chain variable domain or region comprising the amino acid sequences set forth in SEQ ID NO:81 or SEQ ID NO:81 with one, two, or three amino acid additions, deletions, or substitutions, SEQ ID NO:82 or SEQ ID NO:82 with one, two, or three amino acid additions, deletions, or substitutions, and SEQ ID NO:83 or SEQ ID NO:83 with one, two, or three amino acid additions, deletions, or substitutions; or
(xii) a heavy chain variable domain or region comprising the amino acid sequences set forth in SEQ ID NO:89 or SEQ ID NO:89 with one, two, or three amino acid additions, deletions, or substitutions, SEQ ID NO:90 or SEQ ID NO:90 with one, two, or three amino acid additions, deletions, or substitutions, and SEQ ID NO:91 or SEQ ID NO:91 with one, two, or three amino acid additions, deletions, or substitutions.

8-9. (canceled)

10. A chimeric antigen receptor comprising an antigen binding domain, a hinge, a transmembrane domain, and one or more signaling domains, wherein said antigen binding domain comprises an antigen-binding fragment of claim 3.

11. The chimeric antigen receptor of claim 10, wherein said antigen binding domain comprises a scFv having the ability to bind to a mesothelin polypeptide.

12. (canceled)

13. The chimeric antigen receptor of claim 10, wherein said hinge comprises a hinge set forth in FIG. 16.

14. The chimeric antigen receptor of claim 10, wherein said transmembrane domain comprises a transmembrane domain set forth in FIG. 17.

15. The chimeric antigen receptor of claim 10, wherein said chimeric antigen receptor comprises one or more signaling domains set forth in FIG. 18.

16-17. (canceled)

18. A cell engager comprising a first antigen binding domain, a linker, and a second antigen binding domain, wherein said first antigen binding domain comprises an antigen-binding fragment of claim 3.

19. The cell engager of claim 18, wherein said first antigen binding domain comprises a scFv having the ability to bind to a mesothelin polypeptide.

20. (canceled)

21. The cell engager of claim 18, wherein said linker comprises a linker set forth in FIG. 15 or FIG. 16.

22. The cell engager of claim 18, wherein said second antigen binding domain binds to a polypeptide expressed on the surface of T cells.

23. The cell engager of claim 22, wherein said polypeptide expressed on the surface of T cells is a CD3 polypeptide.

24. The cell engager of claim 22, wherein said second antigen binding domain is an antigen binding domain set forth in FIG. 19.

25. The cell engager of claim 18, wherein said second antigen binding domain binds to a polypeptide expressed on the surface of NK cells.

26. The cell engager of claim 25, wherein said polypeptide expressed on the surface of NK cells is a CD16a, NKG2A, NKG2D, NKp30, NKp44, or NKp46 polypeptide.

27. The cell engager of claim 25, wherein said second antigen binding domain is an antigen binding domain set forth in FIG. 20.

28. The cell engager of claim 18, wherein said cell engager comprises a third antigen binding domain.

29. The cell engager of claim 28, wherein said third antigen binding domain binds to a polypeptide expressed on the surface of NK cells.

30. The cell engager of claim 29, wherein said polypeptide expressed on the surface of NK cells is a CD16a, NKG2A, NKG2D, NKp30, NKp44, or NKp46 polypeptide.

31. The cell engager of claim 29, wherein said third antigen binding domain is an antigen binding domain set forth in FIG. 20.

32-50. (canceled)


SEQUENCE LISTING (560 sequences)

SEQ ID NO: 1 - protein, 8 aa, Artificial Sequence
  Description of Artificial Sequence Synthetic peptide
GFTFSSYA

SEQ ID NO: 2 - protein, 7 aa, Artificial Sequence
  Description of Artificial Sequence Synthetic peptide
ISYSGST

SEQ ID NO: 3 - protein, 15 aa, Artificial Sequence
  Description of Artificial Sequence Synthetic peptide
AKYYCSGGTCYYFDY

SEQ ID NO: 4 - protein, 25 aa, Artificial Sequence
  Description of Artificial Sequence Synthetic peptide
EVQLVESGGGVVQPGRSLRLSCAAS

SEQ ID NO: 5 - protein, 17 aa, Artificial Sequence
  Description of Artificial Sequence Synthetic peptide
MHWVRQAPGKALEWIGS

SEQ ID NO: 6 - protein, 38 aa, Artificial Sequence
  Description of Artificial Sequence Synthetic polypeptide
YYNPSLKSRVTVSRDNAKNTLYLQMDSLRAEDTAMYYC

SEQ ID NO: 7 - protein, 11 aa, Artificial Sequence
  Description of Artificial Sequence Synthetic peptide
WGQGTLVTVSS

SEQ ID NO: 8 - protein, 121 aa, Artificial Sequence
  Description of Artificial Sequence Synthetic polypeptide
EVQLVESGGGVVQPGRSLRLSCAASGFTFSSYAMHWVRQAPGKALEWIGSISYSGSTYYNPSLKSRVTVSRDNAKNTLYLQMDSLRAEDTAMYYCAKYYCSGGTCYYFDYWGQGTLVTVSS

SEQ ID NO: 9 - protein, 8 aa, Artificial Sequence
  Description of Artificial Sequence Synthetic peptide
GFTFSSYA

SEQ ID NO: 10 - protein, 7 aa, Artificial Sequence
  Description of Artificial Sequence Synthetic peptide
IYTSGST

SEQ ID NO: 11 - protein, 22 aa, Artificial Sequence
  Description of Artificial Sequence Synthetic peptide
ARTPRGRDSSGYYQSPHAFDIW

SEQ ID NO: 12 - protein, 25 aa, Artificial Sequence
  Description of Artificial Sequence Synthetic peptide
EVQLVESGGGVVQPGRSLRLSCAAS

SEQ ID NO: 13 - protein, 17 aa, Artificial Sequence
  Description of Artificial Sequence Synthetic peptide
MHWVRQAPGKGLEWIGY

SEQ ID NO: 14 - protein, 38 aa, Artificial Sequence
  Description of Artificial Sequence Synthetic polypeptide
NYNPSLKSRVTISRDDSTNTLYLQMNSLRAEDTATYYC

SEQ ID NO: 15 - protein, 10 aa, Artificial Sequence
  Description of Artificial Sequence Synthetic peptide
GQGTLVTVSS

SEQ ID NO: 16 - protein, 127 aa, Artificial Sequence
  Description of Artificial Sequence Synthetic polypeptide
EVQLVESGGGVVQPGRSLRLSCAASGFTFSSYAMHWVRQAPGKGLEWIGYIYTSGSTNYNPSLKSRVTISRDDSTNTLYLQMNSLRAEDTATYYCARTPRGRDSSGYYQSPHAFDIWGQGTLVTVSS

SEQ ID NO: 17 - protein, 8 aa, Artificial Sequence
  Description of Artificial Sequence Synthetic peptide
GGSFSGYY

SEQ ID NO: 18 - protein, 7 aa, Artificial Sequence
  Description of Artificial Sequence Synthetic peptide
INHSGST

SEQ ID NO: 19 - protein, 16 aa, Artificial Sequence
  Description of Artificial Sequence Synthetic peptide
ARTYRPHSYYYYGMDV

SEQ ID NO: 20 - protein, 25 aa, Artificial Sequence
  Description of Artificial Sequence Synthetic peptide
KVQLQQWGAGLLKPSETLSLTCAVY

SEQ ID NO: 21 - protein, 17 aa, Artificial Sequence
  Description of Artificial Sequence Synthetic peptide
WSWIRQPPGKGLEWIGE

SEQ ID NO: 22 - protein, 38 aa, Artificial Sequence
  Description of Artificial Sequence Synthetic polypeptide
NYNPSLKSRVTISVDTSKNQFSLKLSSVTAADTALYYC

SEQ ID NO: 23 - protein, 11 aa, Artificial Sequence
  Description of Artificial Sequence Synthetic peptide
WGQGTTVTVSS

SEQ ID NO: 24 - protein, 122 aa, Artificial Sequence
  Description of Artificial Sequence Synthetic polypeptide
KVQLQQWGAGLLKPSETLSLTCAVYGGSFSGYYWSWIRQPPGKGLEWIGEINHSGSTNYNPSLKSRVTISVDTSKNQFSLKLSSVTAADTALYYCARTYRPHSYYYYGMDVWGQGTTVTVSS

SEQ ID NO: 25 - protein, 8 aa, Artificial Sequence
  Description of Artificial Sequence Synthetic peptide
GFTFSSYA

SEQ ID NO: 26 - protein, 8 aa, Artificial Sequence
  Description of Artificial Sequence Synthetic peptide
ISGNSLYK

SEQ ID NO: 27 - protein, 8 aa, Artificial Sequence
  Description of Artificial Sequence Synthetic peptide
TRHWAVGN

SEQ ID NO: 28 - protein, 25 aa, Artificial Sequence
  Description of Artificial Sequence Synthetic peptide
EVQLVESGGGVVQPGRSLRLSCAAS

SEQ ID NO: 29 - protein, 17 aa, Artificial Sequence
  Description of Artificial Sequence Synthetic peptide
MHWVRQAPGKGLEWVSS

SEQ ID NO: 30 - protein, 38 aa, Artificial Sequence
  Description of Artificial Sequence Synthetic polypeptide
YYADSVKGRFTISRDDSTNTLYLQMNSLRAEDTATYYC

SEQ ID NO: 31 - protein, 11 aa, Artificial Sequence
  Description of Artificial Sequence Synthetic peptide
WGQGTLVTVSS

SEQ ID NO: 32 - protein, 115 aa, Artificial Sequence
  Description of Artificial Sequence Synthetic polypeptide
EVQLVESGGGVVQPGRSLRLSCAASGFTFSSYAMHWVRQAPGKGLEWVSSISGNSLYKYYADSVKGRFTISRDDSTNTLYLQMNSLRAEDTATYYCTRHWAVGNWGQGTLVTVSS

SEQ ID NO: 33 - protein, 8 aa, Artificial Sequence
  Description of Artificial Sequence Synthetic peptide
GLTFSSYA

SEQ ID NO: 34 - protein, 7 aa, Artificial Sequence
  Description of Artificial Sequence Synthetic peptide
IRYDGNT

SEQ ID NO: 35 - protein, 18 aa, Artificial Sequence
  Description of Artificial Sequence Synthetic peptide
ARRRYYDSSGYRGAAFDI

SEQ ID NO: 36 - protein, 25 aa, Artificial Sequence
  Description of Artificial Sequence Synthetic peptide
EVQLVESGGGVVQPGRSLRLSCAAS

SEQ ID NO: 37 - protein, 17 aa, Artificial Sequence
  Description of Artificial Sequence Synthetic peptide
MHWVRQAPGKGLEWIGT

SEQ ID NO: 38 - protein, 38 aa, Artificial Sequence
  Description of Artificial Sequence Synthetic polypeptide
YYNPSLKSLVTISRDDSTNTLYLQMNSLRAEDTATYYC

SEQ ID NO: 39 - protein, 11 aa, Artificial Sequence
  Description of Artificial Sequence Synthetic peptide
WGQGTLVTVSS

SEQ ID NO: 40 - protein, 124 aa, Artificial Sequence
  Description of Artificial Sequence Synthetic polypeptide
EVQLVESGGGVVQPGRSLRLSCAASGLTFSSYAMHWVRQAPGKGLEWIGTIRYDGNTYYNPSLKSLVTISRDDSTNTLYLQMNSLRAEDTATYYCARRRYYDSSGYRGAAFDIWGQGTLVTVSS

SEQ ID NO: 41 - protein, 8 aa, Artificial Sequence
  Description of Artificial Sequence Synthetic peptide
GGSFSGYY

SEQ ID NO: 42 - protein, 7 aa, Artificial Sequence
  Description of Artificial Sequence Synthetic peptide
INHSGST

SEQ ID NO: 43 - protein, 16 aa, Artificial Sequence
  Description of Artificial Sequence Synthetic peptide
ARSYRTVTYYKAYFQH

SEQ ID NO: 44 - protein, 25 aa, Artificial Sequence [sequence omitted]

SEQ ID NO: 45 - protein, 17 aa, Artificial Sequence
  Description of Artificial Sequence Synthetic peptide
WSWIRQPPGKGLEWIGE

SEQ ID NO: 46 - protein, 38 aa, Artificial Sequence
  Description of Artificial Sequence Synthetic polypeptide
NYNPSLKSRVTISVDTSKNQFSLKLSSVTAADTATYYC

SEQ ID NO: 47 - protein, 11 aa, Artificial Sequence
  Description of Artificial Sequence Synthetic peptide
WGQGTLVTVSS

SEQ ID NO: 48 - protein, 122 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTATYYCARSYRTVTYYKAYFQHWGQGTLVTVSS

SEQ ID NO: 49 - protein, 8 aa, Artificial Sequence
  Description of Artificial Sequence Synthetic peptide
GFTFSSYA

SEQ ID NO: 50 - protein, 7 aa, Artificial Sequence
  Description of Artificial Sequence Synthetic peptide
IRYDGNT

SEQ ID NO: 51 - protein, 14 aa, Artificial Sequence
  Description of Artificial Sequence Synthetic peptide
AKYGCSSTSCSFDY

SEQ ID NO: 52 - protein, 25 aa, Artificial Sequence
  Description of Artificial Sequence Synthetic peptide
EVQLVESGGGVVQPGRSLRLSCAAS

SEQ ID NO: 53 - protein, 17 aa, Artificial Sequence
  Description of Artificial Sequence Synthetic peptide
MHWVRQAPGKGLEWIGT

SEQ ID NO: 54 - protein, 38 aa, Artificial Sequence
  Description of Artificial Sequence Synthetic polypeptide
YYNPSLKSLVTISRDDSTNTLYLQMNSLRAEDTALYYC

SEQ ID NO: 55 - protein, 11 aa, Artificial Sequence
  Description of Artificial Sequence Synthetic peptide
WGQGTLVTVSS

SEQ ID NO: 56 - protein, 120 aa, Artificial Sequence
  Description of Artificial Sequence Synthetic polypeptide
EVQLVESGGGVVQPGRSLRLSCAASGFTFSSYAMHWVRQAPGKGLEWIGTIRYDGNTYYNPSLKSLVTISRDDSTNTLYLQMNSLRAEDTALYYCAKYGCSSTSCSFDYWGQGTLVTVSS

SEQ ID NO: 57 - protein, 8 aa, Artificial Sequence
  Description of Artificial Sequence Synthetic peptide
GFTFSSYA

SEQ ID NO: 58 - protein, 7 aa, Artificial Sequence
  Description of Artificial Sequence Synthetic peptide
IRHDGNT

SEQ ID NO: 59 - protein, 14 aa, Artificial Sequence
  Description of Artificial Sequence Synthetic peptide
AKYGCSSISCSFDY

SEQ ID NO: 60 - protein, 25 aa, Artificial Sequence
  Description of Artificial Sequence Synthetic peptide
EVQLVESGGGVVQPGRSLRLSCAAS

SEQ ID NO: 61 - protein, 17 aa, Artificial Sequence
  Description of Artificial Sequence Synthetic peptide
MHWVRQAPGKGLEWIGS

SEQ ID NO: 62 - protein, 38 aa, Artificial Sequence
  Description of Artificial Sequence Synthetic polypeptide
YYNPSLKSRVSVSRDNAKNTLYLQMDSLRAEDTAMYYC

SEQ ID NO: 63 - protein, 11 aa, Artificial Sequence
  Description of Artificial Sequence Synthetic peptide
WGQGTLVTVSS

SEQ ID NO: 64 - protein, 120 aa, Artificial Sequence
  Description of Artificial Sequence Synthetic polypeptide
EVQLVESGGGVVQPGRSLRLSCAASGFTFSSYAMHWVRQAPGKGLEWIGSIRHDGNTYYNPSLKSRVSVSRDNAKNTLYLQMDSLRAEDTAMYYCAKYGCSSISCSFDYWGQGTLVTVSS

SEQ ID NO: 65 - protein, 8 aa, Artificial Sequence
  Description of Artificial Sequence Synthetic peptide
GFTFSSYA

SEQ ID NO: 66 - protein, 8 aa, Artificial Sequence
  Description of Artificial Sequence Synthetic peptide
ISYNGDHT

SEQ ID NO: 67 - protein, 8 aa, Artificial Sequence
  Description of Artificial Sequence Synthetic peptide
TRNYFFDY

SEQ ID NO: 68 - protein, 25 aa, Artificial Sequence
  Description of Artificial Sequence Synthetic peptide
EVQLVESGGGVVQPGRSLRLSCAAS

SEQ ID NO: 69 - protein, 17 aa, Artificial Sequence
  Description of Artificial Sequence Synthetic peptide
MHWVRQAPGKGLEWVSG

SEQ ID NO: 70 - protein, 38 aa, Artificial Sequence
  Description of Artificial Sequence Synthetic polypeptide
YYADSVKGRSTVSRDNAKNTLYLQMDSLRPEDTAMYYC

SEQ ID NO: 71 - protein, 11 aa, Artificial Sequence
  Description of Artificial Sequence Synthetic peptide
WGQGTLVTVSS

SEQ ID NO: 72 - protein, 115 aa, Artificial Sequence
  Description of Artificial Sequence Synthetic polypeptide
EVQLVESGGGVVQPGRSLRLSCAASGFTFSSYAMHWVRQAPGKGLEWVSGISYNGDHTYYADSVKGRSTVSRDNAKNTLYLQMDSLRPEDTAMYYCTRNYFFDYWGQGTLVTVSS

SEQ ID NO: 73 - protein, 8 aa, Artificial Sequence
  Description of Artificial Sequence Synthetic peptide
GFTFSSYA

SEQ ID NO: 74 - protein, 7 aa, Artificial Sequence
  Description of Artificial Sequence Synthetic peptide
IRHDGNT

SEQ ID NO: 75 - protein, 14 aa, Artificial Sequence
  Description of Artificial Sequence Synthetic peptide
AKYGCSSISCSFDY

SEQ ID NO: 76 - protein, 25 aa, Artificial Sequence
  Description of Artificial Sequence Synthetic peptide
EVQLVESGGGVVQPGRSLRLSCAAS

SEQ ID NO: 77 - protein, 17 aa, Artificial Sequence
  Description of Artificial Sequence Synthetic peptide
MHWVRQAPGKGLEWIGS

SEQ ID NO: 78 - protein, 38 aa, Artificial Sequence; identical to SEQ ID NO: 6
  Description of Artificial Sequence Synthetic polypeptide

SEQ ID NO: 79 - protein, 11 aa, Artificial Sequence
  Description of Artificial Sequence Synthetic peptide
WGQGTLVTVSS

SEQ ID NO: 80 - protein, 120 aa, Artificial Sequence
  Description of Artificial Sequence Synthetic polypeptide
EVQLVESGGGVVQPGRSLRLSCAASGFTFSSYAMHWVRQAPGKGLEWIGSIRHDGNTYYNPSLKSRVTVSRDNAKNTLYLQMDSLRAEDTAMYYCAKYGCSSISCSFDYWGQGTLVTVSS

SEQ ID NO: 81 - protein, 8 aa, Artificial Sequence
  Description of Artificial Sequence Synthetic peptide
GFTFSSYA

SEQ ID NO: 82 - protein, 8 aa, Artificial Sequence
  Description of Artificial Sequence Synthetic peptide
ITRSGGAT

SEQ ID NO: 83 - protein, 9 aa, Artificial Sequence
  Description of Artificial Sequence Synthetic peptide
SRDPRRVDY

SEQ ID NO: 84 - protein, 25 aa, Artificial Sequence
  Description of Artificial Sequence Synthetic peptide
EVQLVESGGGVVQPGRSLRLSCAAS

SEQ ID NO: 85 - protein, 17 aa, Artificial Sequence
  Description of Artificial Sequence Synthetic peptide
MHWVRQAPGKGLEWVSG

SEQ ID NO: 86 - protein, 38 aa, Artificial Sequence
  Description of Artificial Sequence Synthetic polypeptide
FYAGSVKGRFTISRDDSTNTLYLQMNSLRAEDTAVYYC

SEQ ID NO: 87 - protein, 11 aa, Artificial Sequence
  Description of Artificial Sequence Synthetic peptide
WGQGTLVTVSS

SEQ ID NO: 88 - protein, 116 aa, Artificial Sequence
  Description of Artificial Sequence Synthetic polypeptide
EVQLVESGGGVVQPGRSLRLSCAASGFTFSSYAMHWVRQAPGKGLEWVSGITRSGGATFYAGSVKGRFTISRDDSTNTLYLQMNSLRAEDTAVYYCSRDPRRVDYWGQGTLVTVSS

SEQ ID NO: 89 - protein, 8 aa, Artificial Sequence
  Description of Artificial Sequence Synthetic peptide
GFTFSSSW

SEQ ID NO: 90 - protein, 8 aa, Artificial Sequence
  Description of Artificial Sequence Synthetic peptide
LSGRSEKT

SEQ ID NO: 91 - protein, 7 aa, Artificial Sequence
  Description of Artificial Sequence Synthetic peptide
ASGWALV

SEQ ID NO: 92 - protein, 25 aa, Artificial Sequence
  Description of Artificial Sequence Synthetic peptide
EVQLVETGGDLAQPGESLRLSCVAS

SEQ ID NO: 93 - protein, 17 aa, Artificial Sequence
  Description of Artificial Sequence Synthetic peptide
MTWIRQAPGKGLEWVSS

SEQ ID NO: 94 - protein, 38 aa, Artificial Sequence
  Description of Artificial Sequence Synthetic polypeptide
FYADSVKGRFTVSRDNAKNTLYLQMDSLRPEDTATYYC

SEQ ID NO: 95 - protein, 11 aa, Artificial Sequence
  Description of Artificial Sequence Synthetic peptide
WGQGTLVTVSS

SEQ ID NO: 96 - protein, 114 aa, Artificial Sequence
  Description of Artificial Sequence Synthetic polypeptide
EVQLVETGGDLAQPGESLRLSCVASGFTFSSSWMTWIRQAPGKGLEWVSSLSGRSEKTFYADSVKGRFTVSRDNAKNTLYLQMDSLRPEDTATYYCASGWALVWGQGTLVTVSS

SEQ ID NO: 97 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 98 - DNA, 363 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaggccctggagtggattgggagtatttcttatagtgggagcacctactacaatccgtccctcaagagtcgagtcaccgtctccagagacaacgccaagaacacgctgtatctgcaaatggacagtctgagagccgaggacacagccatgtattactgtgcgaaatactactgtagtggcggtacctgctactattttgactactggggccagggcaccctggtcaccgtctcctca

SEQ ID NO: 99 - DNA, 381 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaggggctagagtggattgggtacatctataccagtgggagcaccaactacaacccctccctcaagagtcgagtcaccatctccagagacgattccacgaacacgctgtatctgcaaatgaacagcctgagagccgaggacacagccacgtattactgtgcgagaactccgaggggacgcgatagtagtggttattaccaaagtcctcatgcttttgatatctggggccaaggaaccctggtcaccgtctcctca

SEQ ID NO: 100 - DNA, 366 bp, Artificial Sequence
  Description of Artificial Sequence Synthetic polynucleotide
aaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggccttgtattactgtgcgagaacctacaggcctcactcctactactactacggtatggacgtctggggccaagggaccacggtcaccgtctcctca

SEQ ID NO: 101 - DNA, 345 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaggggctggagtgggtctcgtccattagtggaaatagtctttacaaatattacgcagactcagtgaagggccgattcaccatctccagagacgattccacgaacacgctgtatctgcaaatgaacagcctgagagccgaggacacagccacgtattactgtacgagacattgggcggtcggcaactggggccagggaaccctggtcaccgtctcctca

SEQ ID NO: 102 - DNA, 372 bp, Artificial Sequence
  Description of Artificial Sequence Synthetic polynucleotide
gaggtgcagctggtggagtccgggggaggcgtggtccagcctgggaggtccctgagactctcctgtgcagcctctggactcaccttcagtagctatgctatgcactgggtccgccaggctccagggaaaggcctggagtggattgggaccattcgttatgatgggaacacctactacaacccgtccctcaagagtctagtcaccatttccagagacgattccacgaacacgctgtatctgcaaatgaacagcctgagagccgaggacacagccacgtattactgtgcgagacgccgatactatgatagtagtggttatcggggggctgcttttgatatctggggccaaggcaccctggtcaccgtctcctca

SEQ ID NO: 103 - DNA, 366 bp, Artificial Sequence
  Description of Artificial Sequence Synthetic polynucleotide
caggtgcagctacagcagtggggcgcaggactgttgaagccttcggagaccctgtccctcacctgcgctgtctatggtgggtccttcagtggttactactggagctggatccgccagcccccagggaaaggcctggagtggattggggaaatcaatcatagtggaagcaccaactacaacccgtccctcaagagtcgagtcaccatatcagtagacacgtccaagaaccagttctccctgaagctgagctctgtgaccgccgcggacacagccacgtattactgtgcgagatcttatcgaacggtgacctactacaaggcctacttccagcactggggccagggcaccctggtcaccgtctcctca

SEQ ID NO: 104 - DNA, 360 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaagggtctagagtggattgggaccatccgttatgatgggaacacctactacaacccgtccctcaagagtctagtcaccatctccagagacgattccacgaacacgctgtatctgcaaatgaacagcctgagagccgaggacacagctttatattactgtgcgaaatatgggtgtagtagtaccagctgctcttttgactactggggccagggaaccctggtcaccgtctcctca

SEQ ID NO: 105 - DNA, 360 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaagggcctggagtggattgggagcatccgtcatgatggaaacacctactacaacccgtccctcaagagtcgagtcagcgtctccagagacaacgccaagaacacgctgtatctgcaaatggacagtctgagagccgaggacacagccatgtattactgtgcgaaatatgggtgtagtagtatcagctgctcttttgactactggggccagggcaccctggtcaccgtctcctca

SEQ ID NO: 106 - DNA, 345 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtctctggcattagttataatggcgaccacacatactacgcagactcggtgaagggccgatccaccgtctccagagacaacgccaagaacacgctgtatctgcaaatggacagtctgagacccgaggacacagccatgtattactgtacgaggaattacttctttgactactggggccagggcaccctggtcaccgtctcctca

SEQ ID NO: 107 - DNA, 360 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaagggcctggagtggattgggagcatccgtcatgatggaaacacctactacaacccgtccctcaagagtcgagtcaccgtctccagagacaacgccaagaacacgctgtatctgcaaatggacagtctgagagccgaggacacagccatgtattactgtgcgaaatatgggtgtagtagtatcagctgctcttttgactactggggccagggcaccctggtcaccgtctcctca

SEQ ID NO: 108 - DNA, 348 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtctcaggtattactcgcagcggtggcgccacattctacgcaggctctgtgaagggccgattcaccatctccagagacgattccacgaacacgctgtatctgcaaatgaacagcctgagagccgaggacacagccgtgtattactgttcgagagacccaagacgtgtggactactggggccagggcaccctggtcaccgtctcctca

SEQ ID NO: 109 - DNA, 342 bp, Artificial Sequence
  Description of Artificial Sequence Synthetic polynucleotide
gaggtgcagctggtggagaccgggggagacttggcccagccgggggaatctctgagactctcctgtgttgcttctggattcaccttcagttcgtcttggatgacttggatccgccaggctccagggaaaggcctggagtgggtctcaagtcttagtggtagaagtgaaaagacattctacgcagactcagtgaagggccgattcaccgtctccagagacaacgccaagaacacgctgtatctgcaaatggacagtctgagacccgaggacacagccacgtattactgtgccagtggctgggccctagtctggggccagggcaccctggtcaccgtctcctca

SEQ ID NO: 110 - protein, 15 aa, Artificial Sequence
  Description of Artificial Sequence Synthetic peptide
GGGGSGGSGSGGGGS

SEQ ID NO: 111 - protein, 15 aa, Artificial Sequence
  Description of Artificial Sequence Synthetic peptide
GGGGSGGSGSSGGGS

SEQ ID NO: 112 - protein, 15 aa, Artificial Sequence
  Description of Artificial Sequence Synthetic peptide
GGGGSGGGGSGGGGS

SEQ ID NO: 113 - protein, 15 aa, Artificial Sequence
  Description of Artificial Sequence Synthetic peptide
GGGGSGSGASSGGGS

SEQ ID NO: 114 - protein, 15 aa, Artificial Sequence
  Description of Artificial Sequence Synthetic peptide
GGGGSGSGASGGGGS

SEQ ID NO: 115 - protein, 15 aa, Artificial Sequence
  Description of Artificial Sequence Synthetic peptide
SGGGSGGGGSGGGGS

SEQ ID NO: 116 - protein, 18 aa, Artificial Sequence
  Description of Artificial Sequence Synthetic peptide
GSTSGSGKPGSGEGSTKG

SEQ ID NO: 117 - protein, 19 aa, Artificial Sequence
  Description of Artificial Sequence Synthetic peptide
PNGASQSSSASHTGSAPGS

SEQ ID NO: 118 - protein, 19 aa, Artificial Sequence
  Description of Artificial Sequence Synthetic peptide
PNGASNSGSAPDTSSAPGS

SEQ ID NO: 119 - protein, 19 aa, Artificial Sequence
  Description of Artificial Sequence Synthetic peptide
PNGASHSGSAPNTSSAPGS

SEQ ID NO: 120 - protein, 19 aa, Artificial Sequence
  Description of Artificial Sequence Synthetic peptide
PNGASESGSASKTSSASGS

SEQ ID NO: 121 - protein, 19 aa, Artificial Sequence
  Description of Artificial Sequence Synthetic peptide
PNGASNSGSAPKTGSASGS

SEQ ID NO: 122 - protein, 19 aa, Artificial Sequence
  Description of Artificial Sequence Synthetic peptide
PNGASKSGSASQTSSAPGS

SEQ ID NO: 123 - protein, 19 aa, Artificial Sequence
  Description of Artificial Sequence Synthetic peptide
PNGASHSSSASQTGSAPGS

SEQ ID NO: 124 - protein, 12 aa, Artificial Sequence
  Description of Artificial Sequence Synthetic peptide
PNGASKRSAPGS

SEQ ID NO: 125 - protein, 19 aa, Artificial Sequence
  Description of Artificial Sequence Synthetic peptide
PNGASHSGSAPHTSSASGS

SEQ ID NO: 126 - protein, 15 aa, Artificial Sequence
  Description of Artificial Sequence Synthetic peptide
RGRGRGRGRSRGGGS

SEQ ID NO: 127 - protein, 15 aa, Artificial Sequence
  Description of Artificial Sequence Synthetic peptide
SHGGSHGGGSGGGGS

SEQ ID NO: 128 - protein, 15 aa, Artificial Sequence
  Description of Artificial Sequence Synthetic peptide
EPKSCDKTHTCPPCP

SEQ ID NO: 129 - protein, 15 aa, Artificial Sequence
  Description of Artificial Sequence Synthetic peptide
DKTHTCPPCPAPPVA

SEQ ID NO: 130 - protein, 12 aa, Artificial Sequence
  Description of Artificial Sequence Synthetic peptide
ESKYGPPCPPCP

SEQ ID NO: 131 - protein, 49 aa, Artificial Sequence
  Description of Artificial Sequence Synthetic polypeptide
KPTTTPAPRPPTPAPTIASQPLSLRPEACRPAAGGAVHTRGLDFACDIY

SEQ ID NO: 132 - protein, 39 aa, Artificial Sequence
  Description of Artificial Sequence Synthetic polypeptide
IEVMYPPPYLDNERSNGTIIHVKGKHLCPSPLFPGPSKP

SEQ ID NO: 133 - protein, 5 aa, Artificial Sequence
  Description of Artificial Sequence Synthetic peptide
GGGGS

SEQ ID NO: 134 - protein, 3 aa, Artificial Sequence
  Description of Artificial Sequence Synthetic peptide
AAA

SEQ ID NO: 135 - protein, 21 aa, Artificial Sequence
  Description of Artificial Sequence Synthetic peptide
LCYLLDGILFIYGVILTALFL

SEQ ID NO: 136 - protein, 22 aa, Artificial Sequence
  Description of Artificial Sequence Synthetic peptide
MALIVLGGVAGLLLFIGLGIFF

SEQ ID NO: 137 - protein, 21 aa, Artificial Sequence
  Description of Artificial Sequence Synthetic peptide
IYIWAPLAGTCGVLLLSLVIT

SEQ ID NO: 138 - protein, 27 aa, Artificial Sequence
  Description of Artificial Sequence Synthetic peptide
FWVLVVVGGVLACYSLLVTVAFIIFWV

SEQ ID NO: 139 - protein, 21 aa, Artificial Sequence
  Description of Artificial Sequence Synthetic peptide
FWLPIGCAAFVVVCILGCILI

SEQ ID NO: 140 - protein, 113 aa, Artificial Sequence
  Description of Artificial Sequence Synthetic polypeptide
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 141 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 142 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 143 - protein, 42 aa, Artificial Sequence
  Description of Artificial Sequence Synthetic polypeptide
ALYLLRRDQRLPPDAHKPPGGGSFRTPIQEEQADAHSTLAKI

SEQ ID NO: 144 - protein, 38 aa, Artificial Sequence
  Description of Artificial Sequence Synthetic polypeptide
CWLTKKKYSSSVHDPNGEYMFMRAVNTAKKSRLTDVTL

SEQ ID NO: 145 - protein, 24 aa, Artificial Sequence
  Description of Artificial Sequence Synthetic peptide
LCARPRRSPAQEDGKVYINMPGRG

SEQ ID NO: 146 - protein, 52 aa, Artificial Sequence
  Description of Artificial Sequence Synthetic polypeptide
YFLGRLVPRGRGAAEAATRKQRITETESPYQELQGQRSDVYSDLNTQRPYYK

SEQ ID NO: 147 - protein, 48 aa, Artificial Sequence
  Description of Artificial Sequence Synthetic polypeptide
QRRKYRSNKGESPVEPAEPCHYSCPREEEGSTIPIQEDYRKPEPACSP

SEQ ID NO: 148 - protein, 119 aa, Artificial Sequence [sequence omitted]

SEQ ID NO: 149 - protein, 107 aa, Artificial Sequence
  Description of Artificial Sequence Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 150 - protein, 122 aa, Artificial Sequence
  Description of Artificial Sequence Synthetic polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 151 - protein, 108 aa, Artificial Sequence
  Description of Artificial Sequence Synthetic polypeptide
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 152 - protein, 117 aa, Artificial Sequence
  Description of Artificial Sequence Synthetic polypeptide
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 153 - protein, 108 aa, Artificial Sequence
  Description of Artificial Sequence Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 154 - protein, 119 aa, Artificial Sequence
  Description of Artificial Sequence Synthetic polypeptide
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 155 - protein, 107 aa, Artificial Sequence
  Description of Artificial Sequence Synthetic polypeptide
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 156 - protein, 121 aa, Artificial Sequence
  Description of Artificial Sequence Synthetic polypeptide
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 157 - protein, 110 aa, Artificial Sequence
  Description of Artificial Sequence Synthetic polypeptide
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 158 - protein, 114 aa, Artificial Sequence
  Description of Artificial Sequence Synthetic polypeptide
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 159 - protein, 114 aa, Artificial Sequence
  Description of Artificial Sequence Synthetic polypeptide
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 160 - protein, 107 aa, Artificial Sequence [sequence omitted]

SEQ ID NO: 161 - protein, 114 aa, Artificial Sequence
  Description of Artificial Sequence Synthetic polypeptide
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 162 - protein, 112 aa, Artificial Sequence
  Description of Artificial Sequence Synthetic polypeptide
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 163 - protein, 120 aa, Artificial Sequence
  Description of Artificial Sequence Synthetic polypeptide
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 164 - protein, 107 aa, Artificial Sequence
  Description of Artificial Sequence Synthetic polypeptide
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 165 - protein, 113 aa, Artificial Sequence
  Description of Artificial Sequence Synthetic polypeptide
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 166 - protein, 5 aa, Artificial Sequence
  Description of Artificial Sequence Synthetic peptide
GQAGR

SEQ ID NO: 167 - protein, 8 aa, Artificial Sequence
  Description of Artificial Sequence Synthetic peptide
GGGSGGGG

SEQ ID NO: 168 - protein, 5 aa, Artificial Sequence
  Description of Artificial Sequence Synthetic peptide
SGGGG

SEQ ID NO: 169 - protein, 5 aa, Artificial Sequence
  Description of Artificial Sequence Synthetic peptide
GGGGS

SEQ ID NO: 170 - protein, 30 aa, Artificial Sequence
  Description of Artificial Sequence Synthetic polypeptide
GGGGSGGGGSGGGGSGGGGSGGGGSGGGGS

SEQ ID NO: 171 - protein, 8 aa, Artificial Sequence
  Description of Artificial Sequence Synthetic peptide
GFTFSDYY

SEQ ID NO: 172 - protein, 8 aa, Artificial Sequence
  Description of Artificial Sequence Synthetic peptide
GYTFTSYY

SEQ ID NO: 173 - protein, 8 aa, Artificial Sequence
  Description of Artificial Sequence Synthetic peptide
GYTFTGYY

SEQ ID NO: 174 - protein, 8 aa, Artificial Sequence
  Description of Artificial Sequence Synthetic peptide
GFTFSSYW

SEQ ID NO: 175 - protein, 8 aa, Artificial Sequence
  Description of Artificial Sequence Synthetic peptide
GFTFSNSD

SEQ ID NO: 176 - protein, 8 aa, Artificial Sequence
  Description of Artificial Sequence Synthetic peptide
GGTFSSYA

SEQ ID NO: 177 - protein, 8 aa, Artificial Sequence
  Description of Artificial Sequence Synthetic peptide
GFTFDDYA

SEQ ID NO: 178 - protein, 8 aa, Artificial Sequence
  Description of Artificial Sequence Synthetic peptide
GFTFSDYY

SEQ ID NO: 179 - protein, 8 aa, Artificial Sequence
  Description of Artificial Sequence Synthetic peptide
GFTFSDHY

SEQ ID NO: 180 - protein, 8 aa, Artificial Sequence
  Description of Artificial Sequence Synthetic peptide
GGSFSGYY

SEQ ID NO: 181 - protein, 8 aa, Artificial Sequence
  Description of Artificial Sequence Synthetic peptide
INPNSGGT

SEQ ID NO: 182 - protein, 8 aa, Artificial Sequence
  Description of Artificial Sequence Synthetic peptide
ISAYNGNT

SEQ ID NO: 183 - protein, 7 aa, Artificial Sequence
  Description of Artificial Sequence Synthetic peptide
ISSSGST

SEQ ID NO: 184 - protein, 7 aa, Artificial Sequence
  Description of Artificial Sequence Synthetic peptide
IGTAGDT

SEQ ID NO: 185 - protein, 8 aa, Artificial Sequence
  Description of Artificial Sequence Synthetic peptide
ISSSSSYI

SEQ ID NO: 186 - protein, 8 aa, Artificial Sequence
  Description of Artificial Sequence Synthetic peptide
ISGSGGST

SEQ ID NO: 187 - protein, 8 aa, Artificial Sequence
  Description of Artificial Sequence Synthetic peptide
ISYDGSNK

SEQ ID NO: 188 - protein, 7 aa, Artificial Sequence
  Description of Artificial Sequence Synthetic peptide
IYSGGST

SEQ ID NO: 189 - protein, 7 aa, Artificial Sequence
  Description of Artificial Sequence Synthetic peptide
IYHSGST

SEQ ID NO: 190 - protein, 8 aa, Artificial Sequence
  Description of Artificial Sequence Synthetic peptide
ISGSGGST

SEQ ID NO: 191 - protein, 15 aa, Artificial Sequence
  Description of Artificial Sequence Synthetic peptide
ARYYCSGGTCYYFDY

SEQ ID NO: 192 - protein, 15 aa, Artificial Sequence
  Description of Artificial Sequence Synthetic peptide
AAYYCSGGTCYYFDY

SEQ ID NO: 193 - protein, 16 aa, Artificial Sequence
  Description of Artificial Sequence Synthetic peptide
AKDYYCSGGTCYYFDY

SEQ ID NO: 194 - protein, 15 aa, Artificial Sequence
  Description of Artificial Sequence Synthetic peptide
VRYYCSGGTCYYFDY

SEQ ID NO: 195 - protein, 15 aa, Artificial Sequence
  Description of Artificial Sequence Synthetic peptide
SRYYCSGGTCYYFDY

SEQ ID NO: 196 - protein, 15 aa, Artificial Sequence
  Description of Artificial Sequence Synthetic peptide
AKYYCSGGTCYYFDY

SEQ ID NO: 197 - protein, 15 aa, Artificial Sequence
  Description of Artificial Sequence Synthetic peptide
KKYYCSGGTCYYFDY

SEQ ID NO: 198 - protein, 16 aa, Artificial Sequence
  Description of Artificial Sequence Synthetic peptide
AKDYYCSGGTCYYFDY

SEQ ID NO: 199 - protein, 16 aa, Artificial Sequence
  Description of Artificial Sequence Synthetic peptide
ARIYYCSGGTCYYFDY

SEQ ID NO: 200 - protein, 15 aa, Artificial Sequence
  Description of Artificial Sequence Synthetic peptide
ATYYCSGGTCYYFDY

SEQ ID NO: 201 - protein, 8 aa, Artificial Sequence
  Description of Artificial Sequence Synthetic peptide
GYTFTGYY

SEQ ID NO: 202 - protein, 8 aa, Artificial Sequence
  Description of Artificial Sequence Synthetic peptide
GYTFTSYA

SEQ ID NO: 203 - protein, 8 aa, Artificial Sequence
  Description of Artificial Sequence Synthetic peptide
GYTFTSYG

SEQ ID NO: 204 - protein, 8 aa, Artificial Sequence
  Description of Artificial Sequence Synthetic peptide
GFTFTSSA

SEQ ID NO: 205 - protein, 8 aa, Artificial Sequence
  Description of Artificial Sequence Synthetic peptide
GFTFSSYW

SEQ ID NO: 206 - protein, 8 aa, Artificial Sequence
  Description of Artificial Sequence Synthetic peptide
GFTFDDYA

SEQ ID NO: 207 - protein, 8 aa, Artificial Sequence
  Description of Artificial Sequence Synthetic peptide
GFTFSSYD

SEQ ID NO: 208 - protein, 8 aa, Artificial Sequence
  Description of Artificial Sequence Synthetic peptide
GFTFSSYG

SEQ ID NO: 209 - protein, 8 aa, Artificial Sequence
  Description of Artificial Sequence Synthetic peptide
GFTFSSSA

SEQ ID NO: 210 - protein, 8 aa, Artificial Sequence
  Description of Artificial Sequence Synthetic peptide
GFTFSDHY

SEQ ID NO: 211 - protein, 8 aa, Artificial Sequence
  Description of Artificial Sequence Synthetic peptide
INPNSGGT

SEQ ID NO: 212 - protein, 8 aa, Artificial Sequence
  Description of Artificial Sequence Synthetic peptide
ISAYNGNT

SEQ ID NO: 213 - protein, 7 aa, Artificial Sequence
  Description of Artificial Sequence Synthetic peptide
ISSSGST

SEQ ID NO: 214 - protein, 7 aa, Artificial Sequence
  Description of Artificial Sequence Synthetic peptide
IGTAGDT

SEQ ID NO: 215 - protein, 8 aa, Artificial Sequence
  Description of Artificial Sequence Synthetic peptide
ISSSSSYI

SEQ ID NO: 216 - protein, 8 aa, Artificial Sequence
  Description of Artificial Sequence Synthetic peptide
ISGSGGST

SEQ ID NO: 217 - protein, 8 aa, Artificial Sequence
  Description of Artificial Sequence Synthetic peptide
ISYDGSNK

SEQ ID NO: 218 - protein, 7 aa, Artificial Sequence
  Description of Artificial Sequence Synthetic peptide
IYSGGST

SEQ ID NO: 219 - protein, 7 aa, Artificial Sequence
  Description of Artificial Sequence Synthetic peptide
IYHSGST

SEQ ID NO: 220 - protein, 8 aa, Artificial Sequence
  Description of Artificial Sequence Synthetic peptide
ISGSGGST

SEQ ID NO: 221 - protein, 22 aa, Artificial Sequence
  Description of Artificial Sequence Synthetic peptide
ARRTPRGRDSSGYYQSPHAFDI

SEQ ID NO: 222 - protein, 21 aa, Artificial Sequence
  Description of Artificial Sequence Synthetic peptide
ATTPRGRDSSGYYQSPHAFDI

SEQ ID NO: 223 - protein, 21 aa, Artificial Sequence
  Description of Artificial Sequence Synthetic peptide
AATPRGRDSSGYYQSPHAFDI

SEQ ID NO: 224 - protein, 21 aa, Artificial Sequence
  Description of Artificial Sequence Synthetic peptide
VRTPRGRDSSGYYQSPHAFDI

SEQ ID NO: 225 - protein, 22 aa, Artificial Sequence
  Description of Artificial Sequence Synthetic peptide
AHRTPRGRDSSGYYQSPHAFDI

SEQ ID NO: 226 - protein, 22 aa, Artificial Sequence
  Description of Artificial Sequence Synthetic peptide
ARITPRGRDSSGYYQSPHAFDI

SEQ ID NO: 227 - protein, 22 aa, Artificial Sequence
  Description of Artificial Sequence Synthetic peptide
AKDTPRGRDSSGYYQSPHAFDI

SEQ ID NO: 228 - protein, 21 aa, Artificial Sequence
  Description of Artificial Sequence Synthetic peptide
TTTPRGRDSSGYYQSPHAFDI

SEQ ID NO: 229 - protein, 21 aa, Artificial Sequence
  Description of Artificial Sequence Synthetic peptide
SRTPRGRDSSGYYQSPHAFDI

SEQ ID NO: 230 - protein, 21 aa, Artificial Sequence
  Description of Artificial Sequence Synthetic peptide
AKTPRGRDSSGYYQSPHAFDI

SEQ ID NO: 231 - protein, 8 aa, Artificial Sequence
  Description of Artificial Sequence Synthetic peptide
GYTFTGYY

SEQ ID NO: 232 - protein, 8 aa, Artificial Sequence
  Description of Artificial Sequence Synthetic peptide
GYTFTSYA

SEQ ID NO: 233 - protein, 8 aa, Artificial Sequence
  Description of Artificial Sequence Synthetic peptide
GYTFTSYG

SEQ ID NO: 234 - protein, 8 aa, Artificial Sequence
  Description of Artificial Sequence Synthetic peptide
GFTFTSSA

SEQ ID NO: 235 - protein, 8 aa, Artificial Sequence
  Description of Artificial Sequence Synthetic peptide
GFTFSSYW

SEQ ID NO: 236 - protein, 8 aa, Artificial Sequence
  Description of Artificial Sequence Synthetic peptide
GFTFDDYA

SEQ ID NO: 237 - protein, 8 aa, Artificial Sequence
  Description of Artificial Sequence Synthetic peptide
GFTFSSYA

SEQ ID NO: 238 - protein, 8 aa, Artificial Sequence
  Description of Artificial Sequence Synthetic peptide
GFTFSSYG

SEQ ID NO: 239 - protein, 8 aa, Artificial Sequence
  Description of Artificial Sequence Synthetic peptide
GFTFSSSA

SEQ ID NO: 240 - protein, 8 aa, Artificial Sequence
  Description of Artificial Sequence Synthetic peptide
GFTFSDHY

SEQ ID NO: 241 - protein, 8 aa, Artificial Sequence
  Description of Artificial Sequence Synthetic peptide
INPNSGGT

SEQ ID NO: 242 - protein, 8 aa, Artificial Sequence
  Description of Artificial Sequence Synthetic peptide
ISAYNGNT

SEQ ID NO: 243 - protein, 7 aa, Artificial Sequence
  Description of Artificial Sequence Synthetic peptide
ISSSGST

SEQ ID NO: 244 - protein, 7 aa, Artificial Sequence
  Description of Artificial Sequence Synthetic peptide
IGTAGDT

SEQ ID NO: 245 - protein, 8 aa, Artificial Sequence
  Description of Artificial Sequence Synthetic peptide
ISSSSSYI

SEQ ID NO: 246 - protein, 8 aa, Artificial Sequence
  Description of Artificial Sequence Synthetic peptide
ISGSGGST

SEQ ID NO: 247 - protein, 8 aa, Artificial Sequence
  Description of Artificial Sequence Synthetic peptide
ISYDGSNK

SEQ ID NO: 248 - protein, 7 aa, Artificial Sequence
  Description of Artificial Sequence Synthetic peptide
IYSGGST

SEQ ID NO: 249 - protein, 7 aa, Artificial Sequence
  Description of Artificial Sequence Synthetic peptide
IYHSGST

SEQ ID NO: 250 - protein, 8 aa, Artificial Sequence
  Description of Artificial Sequence Synthetic peptide
ISGSGGST

SEQ ID NO: 251 - protein, 17 aa, Artificial Sequence
  Description of Artificial Sequence Synthetic peptide
ARRTYRPHSYYYYGMDV

SEQ ID NO: 252 - protein, 16 aa, Artificial Sequence
  Description of Artificial Sequence Synthetic peptide
ATTYRPHSYYYYGMDV

SEQ ID NO: 253 - protein, 16 aa, Artificial Sequence
  Description of Artificial Sequence Synthetic peptide
AATYRPHSYYYYGMDV

SEQ ID NO: 254 - protein, 16 aa, Artificial Sequence
  Description of Artificial Sequence Synthetic peptide
VRTYRPHSYYYYGMDV

SEQ ID NO: 255 - protein, 17 aa, Artificial Sequence
  Description of Artificial Sequence Synthetic peptide
AHRTYRPHSYYYYGMDV

SEQ ID NO: 256 - protein, 17 aa, Artificial Sequence
  Description of Artificial Sequence Synthetic peptide
ARITYRPHSYYYYGMDV

SEQ ID NO: 257 - protein, 17 aa, Artificial Sequence
  Description of Artificial Sequence Synthetic peptide
AKDTYRPHSYYYYGMDV

SEQ ID NO: 258 - protein, 16 aa, Artificial Sequence
  Description of Artificial Sequence Synthetic peptide
TTTYRPHSYYYYGMDV

SEQ ID NO: 259 - protein, 16 aa, Artificial Sequence
  Description of Artificial Sequence Synthetic peptide
SRTYRPHSYYYYGMDV

SEQ ID NO: 260 - protein, 16 aa, Artificial Sequence
  Description of Artificial Sequence Synthetic peptide
AKTYRPHSYYYYGMDV

SEQ ID NO: 261 - protein, 8 aa, Artificial Sequence
  Description of Artificial Sequence Synthetic peptide
GYTFTGYY

SEQ ID NO: 262 - protein, 8 aa, Artificial Sequence
  Description of Artificial Sequence Synthetic peptide
GYTFTSYA

SEQ ID NO: 263 - protein, 8 aa, Artificial Sequence
  Description of Artificial Sequence Synthetic peptide
GYTFTSYG

SEQ ID NO: 264 - protein, 8 aa, Artificial Sequence
  Description of Artificial Sequence Synthetic peptide
GFTFTSSA

SEQ ID NO: 265 - protein, 8 aa, Artificial Sequence
  Description of Artificial Sequence Synthetic peptide
GFTFSSYW

SEQ ID NO: 266 - protein, 8 aa, Artificial Sequence
  Description of Artificial Sequence Synthetic peptide
GFTFDDYA

SEQ ID NO: 267 - protein, 8 aa, Artificial Sequence
  Description of Artificial Sequence Synthetic peptide
GFTFSSYD

SEQ ID NO: 268 - protein, 8 aa, Artificial Sequence
  Description of Artificial Sequence Synthetic peptide
GFTFSSYG

SEQ ID NO: 269 - protein, 8 aa, Artificial Sequence
  Description of Artificial Sequence Synthetic peptide
GFTFSSSA

SEQ ID NO: 270 - protein, 8 aa, Artificial Sequence
  Description of Artificial Sequence Synthetic peptide
GFTFSDHY

SEQ ID NO: 271 - protein, 8 aa, Artificial Sequence
  Description of Artificial Sequence Synthetic peptide
INPNSGGT

SEQ ID NO: 272 - protein, 8 aa, Artificial Sequence
  Description of Artificial Sequence Synthetic peptide
ISAYNGNT

SEQ ID NO: 273 - protein, 7 aa, Artificial Sequence
  Description of Artificial Sequence Synthetic peptide
ISSSGST

SEQ ID NO: 274 - protein, 7 aa, Artificial Sequence
  Description of Artificial Sequence Synthetic peptide
IGTAGDT

SEQ ID NO: 275 - protein, 8 aa, Artificial Sequence
  Description of Artificial Sequence Synthetic peptide
ISSSSSYI

SEQ ID NO: 276 - protein, 8 aa, Artificial Sequence
  Description of Artificial Sequence Synthetic peptide
ISGSGGST

SEQ ID NO: 277 - protein, 8 aa, Artificial Sequence
  Description of Artificial Sequence Synthetic peptide
ISYDGSNK

SEQ ID NO: 278 - protein, 7 aa, Artificial Sequence
  Description of Artificial Sequence Synthetic peptide
IYSGGST

SEQ ID NO: 279 - protein, 7 aa, Artificial Sequence
  Description of Artificial Sequence Synthetic peptide
IYHSGST

SEQ ID NO: 280 - protein, 8 aa, Artificial Sequence
  Description of Artificial Sequence Synthetic peptide
ISGSGGST

SEQ ID NO: 281 - protein, 11 aa, Artificial Sequence
  Description of Artificial Sequence Synthetic peptide
ARRTRHWAVGN

SEQ ID NO: 282 - protein, 10 aa, Artificial Sequence
  Description of Artificial Sequence Synthetic peptide
ATTRHWAVGN

SEQ ID NO: 283 - protein, 10 aa, Artificial Sequence
  Description of Artificial Sequence Synthetic peptide
AATRHWAVGN

SEQ ID NO: 284 - protein, 10 aa, Artificial Sequence
  Description of Artificial Sequence Synthetic peptide
VRTRHWAVGN

SEQ ID NO: 285 - protein, 11 aa, Artificial Sequence
  Description of Artificial Sequence Synthetic peptide
AHRTRHWAVGN

SEQ ID NO: 286 - protein, 11 aa, Artificial Sequence
  Description of Artificial Sequence Synthetic peptide
ARITRHWAVGN

SEQ ID NO: 287 - protein, 11 aa, Artificial Sequence
  Description of Artificial Sequence Synthetic peptide
AKDTRHWAVGN

SEQ ID NO: 288 - protein, 10 aa, Artificial Sequence
  Description of Artificial Sequence Synthetic peptide
TTTRHWAVGN

SEQ ID NO: 289 - protein, 10 aa, Artificial Sequence
  Description of Artificial Sequence Synthetic peptide
SRTRHWAVGN

SEQ ID NO: 290 - protein, 10 aa, Artificial Sequence
  Description of Artificial Sequence Synthetic peptide
AKTRHWAVGN

SEQ ID NO: 291 - protein, 8 aa, Artificial Sequence
  Description of Artificial Sequence Synthetic peptide
GYTFTGYY

SEQ ID NO: 292 - protein, 8 aa, Artificial Sequence
  Description of Artificial Sequence Synthetic peptide
GYTFTSYA

SEQ ID NO: 293 - protein, 8 aa, Artificial Sequence
  Description of Artificial Sequence Synthetic peptide
GYTFTSYG

SEQ ID NO: 294 - protein, 8 aa, Artificial Sequence
  Description of Artificial Sequence Synthetic peptide
GFTFTSSA

SEQ ID NO: 295 - protein, 8 aa, Artificial Sequence
  Description of Artificial Sequence Synthetic peptide
GFTFSSYW

SEQ ID NO: 296 - protein, 8 aa, Artificial Sequence
  Description of Artificial Sequence Synthetic peptide
GFTFDDYA

SEQ ID NO: 297 - protein, 8 aa, Artificial Sequence
  Description of Artificial Sequence Synthetic peptide
GFTFSSYA

SEQ ID NO: 298 - protein, 8 aa, Artificial Sequence
  Description of Artificial Sequence Synthetic peptide
GFTFSSYG

SEQ ID NO: 299 - protein, 8 aa, Artificial Sequence
  Description of Artificial Sequence Synthetic peptide
GFTFSSSA

SEQ ID NO: 300 - protein, 8 aa, Artificial Sequence
  Description of Artificial Sequence Synthetic peptide
GFTFSDHY

SEQ ID NO: 301 - protein, 8 aa, Artificial Sequence
  Description of Artificial Sequence Synthetic peptide
INPNSGGT

SEQ ID NO: 302 - protein, 8 aa, Artificial Sequence
  Description of Artificial Sequence Synthetic peptide
ISAYNGNT

SEQ ID NO: 303 - protein, 7 aa, Artificial Sequence
  Description of Artificial Sequence Synthetic peptide
ISSSGST

SEQ ID NO: 304 - protein, 7 aa, Artificial Sequence
  Description of Artificial Sequence Synthetic peptide
IGTAGDT

SEQ ID NO: 305 - protein, 8 aa, Artificial Sequence
  Description of Artificial Sequence Synthetic peptide
ISSSSSYI

SEQ ID NO: 306 - protein, 8 aa, Artificial Sequence
  Description of Artificial Sequence Synthetic peptide
ISGSGGST

SEQ ID NO: 307 - protein, 8 aa, Artificial Sequence
  Description of Artificial Sequence Synthetic peptide
ISYDGSNK

SEQ ID NO: 308 - protein, 7 aa, Artificial Sequence
  Description of Artificial Sequence Synthetic peptide
IYSGGST

SEQ ID NO: 309 - protein, 7 aa, Artificial Sequence
  Description of Artificial Sequence Synthetic peptide
IYHSGST

SEQ ID NO: 310 - protein, 8 aa, Artificial Sequence
  Description of Artificial Sequence Synthetic peptide
ISGSGGST

SEQ ID NO: 311 - protein, 19 aa, Artificial Sequence
  Description of Artificial Sequence Synthetic peptide
ARRRRYYDSSGYRGAAFDI

SEQ ID NO: 312 - protein, 18 aa, Artificial Sequence
  Description of Artificial Sequence Synthetic peptide
ATRRYYDSSGYRGAAFDI

SEQ ID NO: 313 - protein, 18 aa, Artificial Sequence
  Description of Artificial Sequence Synthetic peptide
AARRYYDSSGYRGAAFDI

SEQ ID NO: 314 - protein, 18 aa, Artificial Sequence
  Description of Artificial Sequence Synthetic peptide
VRRRYYDSSGYRGAAFDI

SEQ ID NO: 315 - protein, 19 aa, Artificial Sequence
  Description of Artificial Sequence Synthetic peptide
AHRRRYYDSSGYRGAAFDI

SEQ ID NO: 316 - protein, 19 aa, Artificial Sequence
  Description of Artificial Sequence Synthetic peptide
ARIRRYYDSSGYRGAAFDI

SEQ ID NO: 317 - protein, 19 aa, Artificial Sequence
  Description of Artificial Sequence Synthetic peptide
AKDRRYYDSSGYRGAAFDI

SEQ ID NO: 318 - protein, 18 aa, Artificial Sequence
  Description of Artificial Sequence Synthetic peptide
TTRRYYDSSGYRGAAFDI

SEQ ID NO: 319 - protein, 18 aa, Artificial Sequence
  Description of Artificial Sequence Synthetic peptide
SRRRYYDSSGYRGAAFDI

SEQ ID NO: 320 - protein, 18 aa, Artificial Sequence
  Description of Artificial Sequence Synthetic peptide
AKRRYYDSSGYRGAAFDI

SEQ ID NO: 321 - protein, 8 aa, Artificial Sequence
  Description of Artificial Sequence Synthetic peptide
GYTFTGYY

SEQ ID NO: 322 - protein, 8 aa, Artificial Sequence
  Description of Artificial Sequence Synthetic peptide
GYTFTSYA

SEQ ID NO: 323 - protein, 8 aa, Artificial Sequence
  Description of Artificial Sequence Synthetic peptide
GYTFTSYG

SEQ ID NO: 324 - protein, 8 aa, Artificial Sequence
  Description of Artificial Sequence Synthetic peptide
GFTFTSSA

SEQ ID NO: 325 - protein, 8 aa, Artificial Sequence
  Description of Artificial Sequence Synthetic peptide
GFTFSSYW

SEQ ID NO: 326 - protein, 8 aa, Artificial Sequence
  Description of Artificial Sequence Synthetic peptide
GFTFDDYA

SEQ ID NO: 327 - protein, 8 aa, Artificial Sequence
  Description of Artificial Sequence Synthetic peptide
GFTFSSYA

SEQ ID NO: 328 - protein, 8 aa, Artificial Sequence
  Description of Artificial Sequence Synthetic peptide
GFTFSSYG

SEQ ID NO: 329 - protein, 8 aa, Artificial Sequence
  Description of Artificial Sequence Synthetic peptide
GFTFSSSA

SEQ ID NO: 330 - protein, 8 aa, Artificial Sequence
  Description of Artificial Sequence Synthetic peptide
GFTFSDHY

SEQ ID NO: 331 - protein, 8 aa, Artificial Sequence
  Description of Artificial Sequence Synthetic peptide
INPNSGGT

SEQ ID NO: 332 - protein, 8 aa, Artificial Sequence
  Description of Artificial Sequence Synthetic peptide
ISAYNGNT

SEQ ID NO: 333 - protein, 7 aa, Artificial Sequence
  Description of Artificial Sequence Synthetic peptide
ISSSGST

SEQ ID NO: 334 - protein, 7 aa, Artificial Sequence
  Description of Artificial Sequence Synthetic peptide
IGTAGDT

SEQ ID NO: 335 - protein, 8 aa, Artificial Sequence
  Description of Artificial Sequence Synthetic peptide
ISSSSSYI

SEQ ID NO: 336 - protein, 8 aa, Artificial Sequence
  Description of Artificial Sequence Synthetic peptide
ISGSGGST

SEQ ID NO: 337 - protein, 8 aa, Artificial Sequence
  Description of Artificial Sequence Synthetic peptide
ISYDGSNK

SEQ ID NO: 338 - protein, 7 aa, Artificial Sequence
  Description of Artificial Sequence Synthetic peptide
IYSGGST

SEQ ID NO: 339 - protein, 7 aa, Artificial Sequence
  Description of Artificial Sequence Synthetic peptide
IYHSGST

SEQ ID NO: 340 - protein, 8 aa, Artificial Sequence
  Description of Artificial Sequence Synthetic peptide
ISGSGGST

SEQ ID NO: 341 - protein, 17 aa, Artificial Sequence
  Description of Artificial Sequence Synthetic peptide
ARRSYRTVTYYKAYFQH

SEQ ID NO: 342 - protein, 16 aa, Artificial Sequence
  Description of Artificial Sequence Synthetic peptide
ATSYRTVTYYKAYFQH

SEQ ID NO: 343 - protein, 16 aa, Artificial Sequence
  Description of Artificial Sequence Synthetic peptide
AASYRTVTYYKAYFQH

SEQ ID NO: 344 - protein, 16 aa, Artificial Sequence
  Description of Artificial Sequence Synthetic peptide
VRSYRTVTYYKAYFQH

SEQ ID NO: 345 - protein, 17 aa, Artificial Sequence
  Description of Artificial Sequence Synthetic peptide
AHRSYRTVTYYKAYFQH

SEQ ID NO: 346 - protein, 17 aa, Artificial Sequence
  Description of Artificial Sequence Synthetic peptide
ARISYRTVTYYKAYFQH

SEQ ID NO: 347 - protein, 17 aa, Artificial Sequence
  Description of Artificial Sequence Synthetic peptide
AKDSYRTVTYYKAYFQH

SEQ ID NO: 348 - protein, 16 aa, Artificial Sequence
  Description of Artificial Sequence Synthetic peptide
TTSYRTVTYYKAYFQH

SEQ ID NO: 349 - protein, 16 aa, Artificial Sequence
  Description of Artificial Sequence Synthetic peptide
SRSYRTVTYYKAYFQH

SEQ ID NO: 350 - protein, 16 aa, Artificial Sequence
  Description of Artificial Sequence Synthetic peptide
AKSYRTVTYYKAYFQH

SEQ ID NO: 351 - protein, 8 aa, Artificial Sequence
  Description of Artificial Sequence Synthetic peptide
GYTFTGYY

SEQ ID NO: 352 - protein, 8 aa, Artificial Sequence
  Description of Artificial Sequence Synthetic peptide
GYTFTSYA

SEQ ID NO: 353 - protein, 8 aa, Artificial Sequence
  Description of Artificial Sequence Synthetic peptide
GYTFTSYG

SEQ ID NO: 354 - protein, 8 aa, Artificial Sequence
  Description of Artificial Sequence Synthetic peptide
GFTFTSSA

SEQ ID NO: 355 - protein, 8 aa, Artificial Sequence
  Description of Artificial Sequence Synthetic peptide
GFTFSSYW

SEQ ID NO: 356 - protein, 8 aa, Artificial Sequence
  Description of Artificial Sequence Synthetic peptide
GFTFDDYA

SEQ ID NO: 357 - protein, 8 aa, Artificial Sequence
  Description of Artificial Sequence Synthetic peptide
GFTFSSYD

SEQ ID NO: 358 - protein, 8 aa, Artificial Sequence
  Description of Artificial Sequence Synthetic peptide
GFTFSSYG

SEQ ID NO: 359 - protein, 8 aa, Artificial Sequence
  Description of Artificial Sequence Synthetic peptide
GFTFSSSA

SEQ ID NO: 360 - protein, 8 aa, Artificial Sequence
  Description of Artificial Sequence Synthetic peptide
GFTFSDHY

SEQ ID NO: 361 - protein, 8 aa, Artificial Sequence
  Description of Artificial Sequence Synthetic peptide
INPNSGGT

SEQ ID NO: 362 - protein, 8 aa, Artificial Sequence
  Description of Artificial Sequence Synthetic peptide
ISAYNGNT

SEQ ID NO: 363 - protein, 7 aa, Artificial Sequence
  Description of Artificial Sequence Synthetic peptide
ISSSGST

SEQ ID NO: 364 - protein, 7 aa, Artificial Sequence
  Description of Artificial Sequence Synthetic peptide
IGTAGDT

SEQ ID NO: 365 - protein, 8 aa, Artificial Sequence
  Description of Artificial Sequence Synthetic peptide
ISSSSSYI

SEQ ID NO: 366 - protein, 8 aa, Artificial Sequence
  Description of Artificial Sequence Synthetic peptide
ISGSGGST

SEQ ID NO: 367 - protein, 8 aa, Artificial Sequence
  Description of Artificial Sequence Synthetic peptide
ISYDGSNK

SEQ ID NO: 368 - protein, 7 aa, Artificial Sequence
  Description of Artificial Sequence Synthetic peptide
IYSGGST

SEQ ID NO: 369 - protein, 7 aa, Artificial Sequence
  Description of Artificial Sequence Synthetic peptide
IYHSGST

SEQ ID NO: 370 - protein, 8 aa, Artificial Sequence
  Description of Artificial Sequence Synthetic peptide
ISGSGGST

SEQ ID NO: 371 - protein, 15 aa, Artificial Sequence
  Description of Artificial Sequence Synthetic peptide
ARRYGCSSTSCSFDY

SEQ ID NO: 372 - protein, 14 aa, Artificial Sequence
  Description of Artificial Sequence Synthetic peptide
ATYGCSSTSCSFDY

SEQ ID NO: 373 - protein, 14 aa, Artificial Sequence
  Description of Artificial Sequence Synthetic peptide
AAYGCSSTSCSFDY

SEQ ID NO: 374 - protein, 14 aa, Artificial Sequence
  Description of Artificial Sequence Synthetic peptide
VRYGCSSTSCSFDY

SEQ ID NO: 375 - protein, 15 aa, Artificial Sequence
  Description of Artificial Sequence Synthetic peptide
AHRYGCSSTSCSFDY

SEQ ID NO: 376 - protein, 15 aa, Artificial Sequence
  Description of Artificial Sequence Synthetic peptide
ARIYGCSSTSCSFDY

SEQ ID NO: 377 - protein, 15 aa, Artificial Sequence
  Description of Artificial Sequence Synthetic peptide
AKDYGCSSTSCSFDY

SEQ ID NO: 378 - protein, 14 aa, Artificial Sequence
  Description of Artificial Sequence Synthetic peptide
TTYGCSSTSCSFDY

SEQ ID NO: 379 - protein, 14 aa, Artificial Sequence
  Description of Artificial Sequence Synthetic peptide
SRYGCSSTSCSFDY

SEQ ID NO: 380 - protein, 15 aa, Artificial Sequence
  Description of Artificial Sequence Synthetic peptide
ARKYGCSSTSCSFDY

SEQ ID NO: 381 - protein, 8 aa, Artificial Sequence
  Description of Artificial Sequence Synthetic peptide
GYTFTGYY

SEQ ID NO: 382 - protein, 8 aa, Artificial Sequence
  Description of Artificial Sequence Synthetic peptide
GYTFTSYA

SEQ ID NO: 383 - protein, 8 aa, Artificial Sequence
  Description of Artificial Sequence Synthetic peptide
GYTFTSYG

SEQ ID NO: 384 - protein, 8 aa, Artificial Sequence
  Description of Artificial Sequence Synthetic peptide
GFTFTSSA

SEQ ID NO: 385 - protein, 8 aa, Artificial Sequence
  Description of Artificial Sequence Synthetic peptide
GFTFSSYW

SEQ ID NO: 386 - protein, 8 aa, Artificial Sequence
  Description of Artificial Sequence Synthetic peptide
GFTFDDYA

SEQ ID NO: 387 - protein, 8 aa, Artificial Sequence
  Description of Artificial Sequence Synthetic peptide
GFTFSSYD

SEQ ID NO: 388 - protein, 8 aa, Artificial Sequence
  Description of Artificial Sequence Synthetic peptide
GFTFSSYG

SEQ ID NO: 389 - protein, 8 aa, Artificial Sequence
  Description of Artificial Sequence Synthetic peptide
GFTFSSSA

SEQ ID NO: 390 - protein, 8 aa, Artificial Sequence
  Description of Artificial Sequence Synthetic peptide
GFTFSDHY

SEQ ID NO: 391 - protein, 8 aa, Artificial Sequence
  Description of Artificial Sequence Synthetic peptide
INPNSGGT

SEQ ID NO: 392 - protein, 8 aa, Artificial Sequence
  Description of Artificial Sequence Synthetic peptide
ISAYNGNT

SEQ ID NO: 393 - protein, 7 aa, Artificial Sequence
  Description of Artificial Sequence Synthetic peptide
ISSSGST

SEQ ID NO: 394 - protein, 7 aa, Artificial Sequence
  Description of Artificial Sequence Synthetic peptide
IGTAGDT

SEQ ID NO: 395 - protein, 8 aa, Artificial Sequence
  Description of Artificial Sequence Synthetic peptide
ISSSSSYI

SEQ ID NO: 396 - protein, 8 aa, Artificial Sequence
  Description of Artificial Sequence Synthetic peptide
ISGSGGST

SEQ ID NO: 397 - protein, 8 aa, Artificial Sequence
  Description of Artificial Sequence Synthetic peptide
ISYDGSNK

SEQ ID NO: 398 - protein, 7 aa, Artificial Sequence
  Description of Artificial Sequence Synthetic peptide
IYSGGST

SEQ ID NO: 399 - protein, 7 aa, Artificial Sequence
  Description of Artificial Sequence Synthetic peptide
IYHSGST

SEQ ID NO: 400 - protein, 8 aa, Artificial Sequence
  Description of Artificial Sequence Synthetic peptide
ISGSGGST

SEQ ID NO: 401 - protein, 15 aa, Artificial Sequence
  Description of Artificial Sequence Synthetic peptide
ARRYGCSSISCSFDY

SEQ ID NO: 402 - protein, 14 aa, Artificial Sequence
  Description of Artificial Sequence Synthetic peptide
ATYGCSSISCSFDY

SEQ ID NO: 403 - protein, 14 aa, Artificial Sequence
  Description of Artificial Sequence Synthetic peptide
AAYGCSSISCSFDY

SEQ ID NO: 404 - protein, 14 aa, Artificial Sequence
  Description of Artificial Sequence Synthetic peptide
VRYGCSSISCSFDY

SEQ ID NO: 405 - protein, 15 aa, Artificial Sequence
  Description of Artificial Sequence Synthetic peptide
AHRYGCSSISCSFDY

SEQ ID NO: 406 - protein, 15 aa, Artificial Sequence
  Description of Artificial Sequence Synthetic peptide
ARIYGCSSISCSFDY

SEQ ID NO: 407 - protein, 15 aa, Artificial Sequence
  Description of Artificial Sequence Synthetic peptide
AKDYGCSSISCSFDY

SEQ ID NO: 408 - protein, 14 aa, Artificial Sequence
  Description of Artificial Sequence Synthetic peptide
TTYGCSSISCSFDY

SEQ ID NO: 409 - protein, 14 aa, Artificial Sequence
  Description of Artificial Sequence Synthetic peptide
SRYGCSSISCSFDY

SEQ ID NO: 410 - protein, 15 aa, Artificial Sequence
  Description of Artificial Sequence Synthetic peptide
ARKYGCSSISCSFDY

SEQ ID NO: 411 - protein, 8 aa, Artificial Sequence
  Description of Artificial Sequence Synthetic peptide
GYTFTGYY

SEQ ID NO: 412 - protein, 8 aa, Artificial Sequence
  Description of Artificial Sequence Synthetic peptide
GYTFTSYA

SEQ ID NO: 413 - protein, 8 aa, Artificial Sequence
  Description of Artificial Sequence Synthetic peptide
GYTFTSYG

SEQ ID NO: 414 - protein, 8 aa, Artificial Sequence
  Description of Artificial Sequence Synthetic peptide
GFTFTSSA

SEQ ID NO: 415 - protein, 8 aa, Artificial Sequence
  Description of Artificial Sequence Synthetic peptide
GFTFSSYW

SEQ ID NO: 416 - protein, 8 aa, Artificial Sequence
  Description of Artificial Sequence Synthetic peptide
GFTFDDYA

SEQ ID NO: 417 - protein, 8 aa, Artificial Sequence
  Description of Artificial Sequence Synthetic peptide
GFTFSSYD

SEQ ID NO: 418 - protein, 8 aa, Artificial Sequence
  Description of Artificial Sequence Synthetic peptide
GFTFSSYG

SEQ ID NO: 419 - protein, 8 aa, Artificial Sequence
  Description of Artificial Sequence Synthetic peptide
GFTFSSSA

SEQ ID NO: 420 - protein, 8 aa, Artificial Sequence
  Description of Artificial Sequence Synthetic peptide
GFTFSDHY

SEQ ID NO: 421 - protein, 8 aa, Artificial Sequence
  Description of Artificial Sequence Synthetic peptide
INPNSGGT

SEQ ID NO: 422 - protein, 8 aa, Artificial Sequence
  Description of Artificial Sequence Synthetic peptide
ISAYNGNT

SEQ ID NO: 423 - protein, 7 aa, Artificial Sequence
  Description of Artificial Sequence Synthetic peptide
ISSSGST

SEQ ID NO: 424 - protein, 7 aa, Artificial Sequence
  Description of Artificial Sequence Synthetic peptide
IGTAGDT

SEQ ID NO: 425 - protein, 8 aa, Artificial Sequence
  Description of Artificial Sequence Synthetic peptide
ISSSSSYI

SEQ ID NO: 426 - protein, 8 aa, Artificial Sequence
  Description of Artificial Sequence Synthetic peptide
ISGSGGST

SEQ ID NO: 427 - protein, 8 aa, Artificial Sequence
  Description of Artificial Sequence Synthetic peptide
ISYDGSNK

SEQ ID NO: 428 - protein, 7 aa, Artificial Sequence
  Description of Artificial Sequence Synthetic peptide
IYSGGST

SEQ ID NO: 429 - protein, 7 aa, Artificial Sequence
  Description of Artificial Sequence Synthetic peptide
IYHSGST

SEQ ID NO: 430 - protein, 8 aa, Artificial Sequence
  Description of Artificial Sequence Synthetic peptide
ISGSGGST

SEQ ID NO: 431 - protein, 11 aa, Artificial Sequence
  Description of Artificial Sequence Synthetic peptide
ARRTRNYFFDY

SEQ ID NO: 432 - protein, 10 aa, Artificial Sequence
  Description of Artificial Sequence Synthetic peptide
ATTRNYFFDY

SEQ ID NO: 433 - protein, 10 aa, Artificial Sequence
  Description of Artificial Sequence Synthetic peptide
AATRNYFFDY

SEQ ID NO: 434 - protein, 10 aa, Artificial Sequence
  Description of Artificial Sequence Synthetic peptide
VRTRNYFFDY

SEQ ID NO: 435 - protein, 11 aa, Artificial Sequence
  Description of Artificial Sequence Synthetic peptide
AHRTRNYFFDY

SEQ ID NO: 436 - protein, 11 aa, Artificial Sequence
  Description of Artificial Sequence Synthetic peptide
ARITRNYFFDY

SEQ ID NO: 437 - protein, 11 aa, Artificial Sequence
  Description of Artificial Sequence Synthetic peptide
AKDTRNYFFDY

SEQ ID NO: 438 - protein, 10 aa, Artificial Sequence
  Description of Artificial Sequence Synthetic peptide
TTTRNYFFDY

SEQ ID NO: 439 - protein, 10 aa, Artificial Sequence
  Description of Artificial Sequence Synthetic peptide
SRTRNYFFDY

SEQ ID NO: 440 - protein, 10 aa, Artificial Sequence
  Description of Artificial Sequence Synthetic peptide
AKTRNYFFDY

SEQ ID NO: 441 - protein, 8 aa, Artificial Sequence
  Description of Artificial Sequence Synthetic peptide
GYTFTGYY

SEQ ID NO: 442 - protein, 8 aa, Artificial Sequence
  Description of Artificial Sequence Synthetic peptide
GYTFTSYA

SEQ ID NO: 443 - protein, 8 aa, Artificial Sequence
  Description of Artificial Sequence Synthetic peptide
GYTFTSYG

SEQ ID NO: 444 - protein, 8 aa, Artificial Sequence
  Description of Artificial Sequence Synthetic peptide
GFTFTSSA

SEQ ID NO: 445 - protein, 8 aa, Artificial Sequence
  Description of Artificial Sequence Synthetic peptide
GFTFSSYW

SEQ ID NO: 446 - protein, 8 aa, Artificial Sequence
  Description of Artificial Sequence Synthetic peptide
GFTFDDYA

SEQ ID NO: 447 - protein, 8 aa, Artificial Sequence
  Description of Artificial Sequence Synthetic peptide
GFTFSSYD

SEQ ID NO: 448 - protein, 8 aa, Artificial Sequence
  Description of Artificial Sequence Synthetic peptide
GFTFSSYG

SEQ ID NO: 449 - protein, 8 aa, Artificial Sequence
  Description of Artificial Sequence Synthetic peptide
GFTFSSSA

SEQ ID NO: 450 - protein, 8 aa, Artificial Sequence
  Description of Artificial Sequence Synthetic peptide
GFTFSDHY

SEQ ID NO: 451 - protein, 8 aa, Artificial Sequence
  Description of Artificial Sequence Synthetic peptide
INPNSGGT

SEQ ID NO: 452 - protein, 8 aa, Artificial Sequence
  Description of Artificial Sequence Synthetic peptide
ISAYNGNT

SEQ ID NO: 453 - protein, 7 aa, Artificial Sequence
  Description of Artificial Sequence Synthetic peptide
ISSSGST

SEQ ID NO: 454 - protein, 7 aa, Artificial Sequence
  Description of Artificial Sequence Synthetic peptide
IGTAGDT

SEQ ID NO: 455 - protein, 8 aa, Artificial Sequence
  Description of Artificial Sequence Synthetic peptide
ISSSSSYI

SEQ ID NO: 456 - protein, 8 aa, Artificial Sequence
  Description of Artificial Sequence Synthetic peptide
ISGSGGST

SEQ ID NO: 457 - protein, 8 aa, Artificial Sequence
  Description of Artificial Sequence Synthetic peptide
ISYDGSNK

SEQ ID NO: 458 - protein, 7 aa, Artificial Sequence
  Description of Artificial Sequence Synthetic peptide
IYSGGST

SEQ ID NO: 459 - protein, 7 aa, Artificial Sequence
  Description of Artificial Sequence Synthetic peptide
IYHSGST

SEQ ID NO: 460 - protein, 8 aa, Artificial Sequence
  Description of Artificial Sequence Synthetic peptide
ISGSGGST

SEQ ID NO: 461 - protein, 15 aa, Artificial Sequence
  Description of Artificial Sequence Synthetic peptide
ARRYGCSSISCSFDY

SEQ ID NO: 462 - protein, 14 aa, Artificial Sequence
  Description of Artificial Sequence Synthetic peptide
ATYGCSSISCSFDY

SEQ ID NO: 463 - protein, 14 aa, Artificial Sequence
  Description of Artificial Sequence Synthetic peptide
AAYGCSSISCSFDY

SEQ ID NO: 464 - protein, 14 aa, Artificial Sequence
  Description of Artificial Sequence Synthetic peptide
VRYGCSSISCSFDY

SEQ ID NO: 465 - protein, 15 aa, Artificial Sequence
  Description of Artificial Sequence Synthetic peptide
AHRYGCSSISCSFDY

SEQ ID NO: 466 - protein, 15 aa, Artificial Sequence
  Description of Artificial Sequence Synthetic peptide
ARIYGCSSISCSFDY

SEQ ID NO: 467 - protein, 15 aa, Artificial Sequence
  Description of Artificial Sequence Synthetic peptide
AKDYGCSSISCSFDY

SEQ ID NO: 468 - protein, 14 aa, Artificial Sequence
  Description of Artificial Sequence Synthetic peptide
TTYGCSSISCSFDY

SEQ ID NO: 469 - protein, 14 aa, Artificial Sequence
  Description of Artificial Sequence Synthetic peptide
SRYGCSSISCSFDY

SEQ ID NO: 470 - protein, 15 aa, Artificial Sequence
  Description of Artificial Sequence Synthetic peptide
ARKYGCSSISCSFDY

SEQ ID NO: 471 - protein, 8 aa, Artificial Sequence
  Description of Artificial Sequence Synthetic peptide
GYTFTGYY

SEQ ID NO: 472 - protein, 8 aa, Artificial Sequence
  Description of Artificial Sequence Synthetic peptide
GYTFTSYA

SEQ ID NO: 473 - protein, 8 aa, Artificial Sequence
  Description of Artificial Sequence Synthetic peptide
GYTFTSYG

SEQ ID NO: 474 - protein, 8 aa, Artificial Sequence
  Description of Artificial Sequence Synthetic peptide
GFTFTSSA

SEQ ID NO: 475 - protein, 8 aa, Artificial Sequence
  Description of Artificial Sequence Synthetic peptide
GFTFSSYW

SEQ ID NO: 476 - protein, 8 aa, Artificial Sequence
  Description of Artificial Sequence Synthetic peptide
GFTFDDYA

SEQ ID NO: 477 - protein, 8 aa, Artificial Sequence
  Description of Artificial Sequence Synthetic peptide
GFTFSSYD

SEQ ID NO: 478 - protein, 8 aa, Artificial Sequence
  Description of Artificial Sequence Synthetic peptide
GFTFSSYG

SEQ ID NO: 479 - protein, 8 aa, Artificial Sequence
  Description of Artificial Sequence Synthetic peptide
GFTFSSSA

SEQ ID NO: 480 - protein, 8 aa, Artificial Sequence
  Description of Artificial Sequence Synthetic peptide
GFTFSDHY

SEQ ID NO: 481 - protein, 8 aa, Artificial Sequence
  Description of Artificial Sequence Synthetic peptide
INPNSGGT

SEQ ID NO: 482 - protein, 8 aa, Artificial Sequence
  Description of Artificial Sequence Synthetic peptide
ISAYNGNT

SEQ ID NO: 483 - protein, 7 aa, Artificial Sequence
  Description of Artificial Sequence Synthetic peptide
ISSSGST

SEQ ID NO: 484 - protein, 7 aa, Artificial Sequence
  Description of Artificial Sequence Synthetic peptide
IGTAGDT

SEQ ID NO: 485 - protein, 8 aa, Artificial Sequence
  Description of Artificial Sequence Synthetic peptide
ISSSSSYI

SEQ ID NO: 486 - protein, 8 aa, Artificial Sequence
  Description of Artificial Sequence Synthetic peptide
ISGSGGST

SEQ ID NO: 487 - protein, 8 aa, Artificial Sequence
  Description of Artificial Sequence Synthetic peptide
ISYDGSNK

SEQ ID NO: 488 - protein, 7 aa, Artificial Sequence
  Description of Artificial Sequence Synthetic peptide
IYSGGST

SEQ ID NO: 489 - protein, 7 aa, Artificial Sequence
  Description of Artificial Sequence Synthetic peptide
IYHSGST

SEQ ID NO: 490 - protein, 8 aa, Artificial Sequence
  Description of Artificial Sequence Synthetic peptide
ISGSGGST

SEQ ID NO: 491 - protein, 12 aa, Artificial Sequence
  Description of Artificial Sequence Synthetic peptide
ARRSRDPRRVDY

SEQ ID NO: 492 - protein, 11 aa, Artificial Sequence
  Description of Artificial Sequence Synthetic peptide
ATSRDPRRVDY

SEQ ID NO: 493 - protein, 11 aa, Artificial Sequence
  Description of Artificial Sequence Synthetic peptide
AASRDPRRVDY

SEQ ID NO: 494 - protein, 11 aa, Artificial Sequence
  Description of Artificial Sequence Synthetic peptide
VRSRDPRRVDY

SEQ ID NO: 495 - protein, 12 aa, Artificial Sequence
  Description of Artificial Sequence Synthetic peptide
AHRSRDPRRVDY

SEQ ID NO: 496 - protein, 12 aa, Artificial Sequence
  Description of Artificial Sequence Synthetic peptide
ARISRDPRRVDY

SEQ ID NO: 497 - protein, 12 aa, Artificial Sequence
  Description of Artificial Sequence Synthetic peptide
AKDSRDPRRVDY

SEQ ID NO: 498 - protein, 11 aa, Artificial Sequence
  Description of Artificial Sequence Synthetic peptide
TTSRDPRRVDY

SEQ ID NO: 499 - protein, 11 aa, Artificial Sequence
  Description of Artificial Sequence Synthetic peptide
SRSRDPRRVDY

SEQ ID NO: 500 - protein, 11 aa, Artificial Sequence
  Description of Artificial Sequence Synthetic peptide
AKSRDPRRVDY

SEQ ID NO: 501 - protein, 8 aa, Artificial Sequence
  Description of Artificial Sequence Synthetic peptide
GYTFTGYY

SEQ ID NO: 502 - protein, 8 aa, Artificial Sequence
  Description of Artificial Sequence Synthetic peptide
GYTFTSYA

SEQ ID NO: 503 - protein, 8 aa, Artificial Sequence
  Description of Artificial Sequence Synthetic peptide
GYTFTSYG

SEQ ID NO: 504 - protein, 8 aa, Artificial Sequence
  Description of Artificial Sequence Synthetic peptide
GFTFTSSA

SEQ ID NO: 505 - protein, 8 aa, Artificial Sequence
  Description of Artificial Sequence Synthetic peptide
GFTFSSYW

SEQ ID NO: 506 - protein, 8 aa, Artificial Sequence
  Description of Artificial Sequence Synthetic peptide
GFTFDDYA

SEQ ID NO: 507 - protein, 8 aa, Artificial Sequence
  Description of Artificial Sequence Synthetic peptide
GFTFSSYA

SEQ ID NO: 508 - protein, 8 aa, Artificial Sequence
  Description of Artificial Sequence Synthetic peptide
GFTFSSYG

SEQ ID NO: 509 - protein, 8 aa, Artificial Sequence
  Description of Artificial Sequence Synthetic peptide
GFTFSSSA

SEQ ID NO: 510 - protein, 8 aa, Artificial Sequence
  Description of Artificial Sequence Synthetic peptide
GFTFSDHY

SEQ ID NO: 511 - protein, 8 aa, Artificial Sequence
  Description of Artificial Sequence Synthetic peptide
INPNSGGT

SEQ ID NO: 512 - protein, 8 aa, Artificial Sequence
  Description of Artificial Sequence Synthetic peptide
ISAYNGNT

SEQ ID NO: 513 - protein, 7 aa, Artificial Sequence
  Description of Artificial Sequence Synthetic peptide
ISSSGST

SEQ ID NO: 514 - protein, 7 aa, Artificial Sequence
  Description of Artificial Sequence Synthetic peptide
IGTAGDT

SEQ ID NO: 515 - protein, 8 aa, Artificial Sequence
  Description of Artificial Sequence Synthetic peptide
ISSSSSYI

SEQ ID NO: 516 - protein, 8 aa, Artificial Sequence
  Description of Artificial Sequence Synthetic peptide
ISGSGGST

SEQ ID NO: 517 - protein, 8 aa, Artificial Sequence
  Description of Artificial Sequence Synthetic peptide
ISYDGSNK

SEQ ID NO: 518 - protein, 7 aa, Artificial Sequence
  Description of Artificial Sequence Synthetic peptide
IYSGGST

SEQ ID NO: 519 - protein, 7 aa, Artificial Sequence
  Description of Artificial Sequence Synthetic peptide
IYHSGST

SEQ ID NO: 520 - protein, 8 aa, Artificial Sequence
  Description of Artificial Sequence Synthetic peptide
ISGSGGST

SEQ ID NO: 521 - protein, 10 aa, Artificial Sequence
  Description of Artificial Sequence Synthetic peptide
ARRASGWALV

SEQ ID NO: 522 - protein, 9 aa, Artificial Sequence
  Description of Artificial Sequence Synthetic peptide
ATASGWALV

SEQ ID NO: 523 - protein, 9 aa, Artificial Sequence
  Description of Artificial Sequence Synthetic peptide
AAASGWALV

SEQ ID NO: 524 - protein, 9 aa, Artificial Sequence
  Description of Artificial Sequence Synthetic peptide
VRASGWALV

SEQ ID NO: 525 - protein, 10 aa, Artificial Sequence
  Description of Artificial Sequence Synthetic peptide
AHRASGWALV

SEQ ID NO: 526 - protein, 10 aa, Artificial Sequence
  Description of Artificial Sequence Synthetic peptide
ARIASGWALV

SEQ ID NO: 527 - protein, 10 aa, Artificial Sequence
  Description of Artificial Sequence Synthetic peptide
AKDASGWALV

SEQ ID NO: 528 - protein, 9 aa, Artificial Sequence
  Description of Artificial Sequence Synthetic peptide
TTASGWALV

SEQ ID NO: 529 - protein, 9 aa, Artificial Sequence
  Description of Artificial Sequence Synthetic peptide
SRASGWALV

SEQ ID NO: 530 - protein, 9 aa, Artificial Sequence
  Description of Artificial Sequence Synthetic peptide
AKASGWALV

SEQ ID NO: 531 - protein, 311 aa, Homo sapiens
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALS

SEQ ID NO: 532 - protein, 47 aa, Artificial Sequence
  Description of Artificial Sequence Synthetic polypeptide
TTTPAPRPPTPAPTIASQPLSLRPEACRPAAGGAVHTRGLDFACDIY

SEQ ID NO: 533 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 534 - protein, 24 aa, Artificial Sequence
  Description of Artificial Sequence Synthetic peptide
IYIWAPLAGTCGVLLLSLVITLYC

SEQ ID NO: 535 - protein, 22 aa, Artificial Sequence
  Description of Artificial Sequence Synthetic peptide
IWAPLAGTCGVLLLSLVITLYC

SEQ ID NO: 536 - protein, 19 aa, Artificial Sequence
  Description of Artificial Sequence Synthetic peptide
IWAPLAGTCGVLLLSLVIT

SEQ ID NO: 537 - protein, 349 aa, Artificial Sequence
  Description of Artificial Sequence Synthetic polypeptide
EVQLVESGGGVVQPGRSLRLSCAASGFTFSSYAMHWVRQAPGKALEWIGSISYSGSTYYNPSLKSRVTVSRDNAKNTLYLQMDSLRAEDTAMYYCAKYYCSGGTCYYFDYWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 538 - DNA, 1050 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaggccctggagtggattgggagtatttcttatagtgggagcacctactacaatccgtccctcaagagtcgagtcaccgtctccagagacaacgccaagaacacgctgtatctgcaaatggacagtctgagagccgaggacacagccatgtattactgtgcgaaatactactgtagtggcggtacctgctactattttgactactggggccagggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 539 - protein, 355 aa, Artificial Sequence
  Description of Artificial Sequence Synthetic polypeptide
EVQLVESGGGVVQPGRSLRLSCAASGFTFSSYAMHWVRQAPGKGLEWIGYIYTSGSTNYNPSLKSRVTISRDDSTNTLYLQMNSLRAEDTATYYCARTPRGRDSSGYYQSPHAFDIWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 540 - DNA, 1068 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaggggctagagtggattgggtacatctataccagtgggagcaccaactacaacccctccctcaagagtcgagtcaccatctccagagacgattccacgaacacgctgtatctgcaaatgaacagcctgagagccgaggacacagccacgtattactgtgcgagaactccgaggggacgcgatagtagtggttattaccaaagtcctcatgcttttgatatctggggccaaggaaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 541 - protein, 350 aa, Artificial Sequence
  Description of Artificial Sequence Synthetic polypeptide
KVQLQQWGAGLLKPSETLSLTCAVYGGSFSGYYWSWIRQPPGKGLEWIGEINHSGSTNYNPSLKSRVTISVDTSKNQFSLKLSSVTAADTALYYCARTYRPHSYYYYGMDVWGQGTTVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 542 - DNA, 1053 bp, Artificial Sequence
  Description of Artificial Sequence Synthetic polynucleotide
aaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggccttgtattactgtgcgagaacctacaggcctcactcctactactactacggtatggacgtctggggccaagggaccacggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 543 - protein, 343 aa, Artificial Sequence
  Description of Artificial Sequence Synthetic polypeptide
EVQLVESGGGVVQPGRSLRLSCAASGFTFSSYAMHWVRQAPGKGLEWVSSISGNSLYKYYADSVKGRFTISRDDSTNTLYLQMNSLRAEDTATYYCTRHWAVGNWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 544 - DNA, 1032 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaggggctggagtgggtctcgtccattagtggaaatagtctttacaaatattacgcagactcagtgaagggccgattcaccatctccagagacgattccacgaacacgctgtatctgcaaatgaacagcctgagagccgaggacacagccacgtattactgtacgagacattgggcggtcggcaactggggccagggaaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 545 - protein, 352 aa, Artificial Sequence
  Description of Artificial Sequence Synthetic polypeptide
EVQLVESGGGVVQPGRSLRLSCAASGLTFSSYAMHWVRQAPGKGLEWIGTIRYDGNTYYNPSLKSLVTISRDDSTNTLYLQMNSLRAEDTATYYCARRRYYDSSGYRGAAFDIWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 546 - DNA, 1059 bp, Artificial Sequence
  Description of Artificial Sequence Synthetic polynucleotide
gaggtgcagctggtggagtccgggggaggcgtggtccagcctgggaggtccctgagactctcctgtgcagcctctggactcaccttcagtagctatgctatgcactgggtccgccaggctccagggaaaggcctggagtggattgggaccattcgttatgatgggaacacctactacaacccgtccctcaagagtctagtcaccatttccagagacgattccacgaacacgctgtatctgcaaatgaacagcctgagagccgaggacacagccacgtattactgtgcgagacgccgatactatgatagtagtggttatcggggggctgcttttgatatctggggccaaggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 547 - protein, 350 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTATYYCARSYRTVTYYKAYFQHWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 548 - DNA, 1053 bp, Artificial Sequence
  Description of Artificial Sequence Synthetic polynucleotide
caggtgcagctacagcagtggggcgcaggactgttgaagccttcggagaccctgtccctcacctgcgctgtctatggtgggtccttcagtggttactactggagctggatccgccagcccccagggaaaggcctggagtggattggggaaatcaatcatagtggaagcaccaactacaacccgtccctcaagagtcgagtcaccatatcagtagacacgtccaagaaccagttctccctgaagctgagctctgtgaccgccgcggacacagccacgtattactgtgcgagatcttatcgaacggtgacctactacaaggcctacttccagcactggggccagggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 549 - protein, 348 aa, Artificial Sequence
  Description of Artificial Sequence Synthetic polypeptide
EVQLVESGGGVVQPGRSLRLSCAASGFTFSSYAMHWVRQAPGKGLEWIGTIRYDGNTYYNPSLKSLVTISRDDSTNTLYLQMNSLRAEDTALYYCAKYGCSSTSCSFDYWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 550 - DNA, 1047 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaagggtctagagtggattgggaccatccgttatgatgggaacacctactacaacccgtccctcaagagtctagtcaccatctccagagacgattccacgaacacgctgtatctgcaaatgaacagcctgagagccgaggacacagctttatattactgtgcgaaatatgggtgtagtagtaccagctgctcttttgactactggggccagggaaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 551 - protein, 348 aa, Artificial Sequence
  Description of Artificial Sequence Synthetic polypeptide
EVQLVESGGGVVQPGRSLRLSCAASGFTFSSYAMHWVRQAPGKGLEWIGSIRHDGNTYYNPSLKSRVSVSRDNAKNTLYLQMDSLRAEDTAMYYCAKYGCSSISCSFDYWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 552 - DNA, 1047 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaagggcctggagtggattgggagcatccgtcatgatggaaacacctactacaacccgtccctcaagagtcgagtcagcgtctccagagacaacgccaagaacacgctgtatctgcaaatggacagtctgagagccgaggacacagccatgtattactgtgcgaaatatgggtgtagtagtatcagctgctcttttgactactggggccagggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 553 - protein, 343 aa, Artificial Sequence
  Description of Artificial Sequence Synthetic polypeptide
EVQLVESGGGVVQPGRSLRLSCAASGFTFSSYAMHWVRQAPGKGLEWVSGISYNGDHTYYADSVKGRSTVSRDNAKNTLYLQMDSLRPEDTAMYYCTRNYFFDYWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 554 - DNA, 1032 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtctctggcattagttataatggcgaccacacatactacgcagactcggtgaagggccgatccaccgtctccagagacaacgccaagaacacgctgtatctgcaaatggacagtctgagacccgaggacacagccatgtattactgtacgaggaattacttctttgactactggggccagggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 555 - protein, 348 aa, Artificial Sequence
  Description of Artificial Sequence Synthetic polypeptide
EVQLVESGGGVVQPGRSLRLSCAASGFTFSSYAMHWVRQAPGKGLEWIGSIRHDGNTYYNPSLKSRVTVSRDNAKNTLYLQMDSLRAEDTAMYYCAKYGCSSISCSFDYWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 556 - DNA, 1047 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaagggcctggagtggattgggagcatccgtcatgatggaaacacctactacaacccgtccctcaagagtcgagtcaccgtctccagagacaacgccaagaacacgctgtatctgcaaatggacagtctgagagccgaggacacagccatgtattactgtgcgaaatatgggtgtagtagtatcagctgctcttttgactactggggccagggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 557 - protein, 344 aa, Artificial Sequence
  Description of Artificial Sequence Synthetic polypeptide
EVQLVESGGGVVQPGRSLRLSCAASGFTFSSYAMHWVRQAPGKGLEWVSGITRSGGATFYAGSVKGRFTISRDDSTNTLYLQMNSLRAEDTAVYYCSRDPRRVDYWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 558 - DNA, 1035 bp, Artificial Sequence
  Description of Artificial Sequence Synthetic polynucleotide
gaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtctcaggtattactcgcagcggtggcgccacattctacgcaggctctgtgaagggccgattcaccatctccagagacgattccacgaacacgctgtatctgcaaatgaacagcctgagagccgaggacacagccgtgtattactgttcgagagacccaagacgtgtggactactggggccagggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 559 - protein, 342 aa, Artificial Sequence
  Description of Artificial Sequence Synthetic polypeptide
EVQLVETGGDLAQPGESLRLSCVASGFTFSSSWMTWIRQAPGKGLEWVSSLSGRSEKTFYADSVKGRFTVSRDNAKNTLYLQMDSLRPEDTATYYCASGWALVWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 560 - DNA, 1029 bp, Artificial Sequence
  Description of Artificial Sequence Synthetic polynucleotide
gaggtgcagctggtggagaccgggggagacttggcccagccgggggaatctctgagactctcctgtgttgcttctggattcaccttcagttcgtcttggatgacttggatccgccaggctccagggaaaggcctggagtgggtctcaagtcttagtggtagaagtgaaaagacattctacgcagactcagtgaagggccgattcaccgtctccagagacaacgccaagaacacgctgtatctgcaaatggacagtctgagacccgaggacacagccacgtattactgtgccagtggctgggccctagtctggggccagggcaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa